(19) United States
(12) Patent Application Publication
(10) Pub. No.: US 2024/0140939 A1
(43) Pub. Date: May 2, 2024

(54) COMPOSITIONS AND METHODS FOR REDUCING PHOTO DAMAGE DURING SEQUENCING

(71) Applicants: ILLUMINA CAMBRIDGE LIMITED, Cambridge (GB); ILLUMINA, INC., San Diego, CA (US)

(72) Inventors: Xiaolin WU, Cambridge (GB); Benedict MACKWORTH, Cambridge (GB); Madushani DHARMARWARDANA, Cambridge (GB); Patrick MCCAULEY, Cambridge (GB); Philip STACKHOUSE, Cambridge (GB); Xiaohai LIU, Cambridge (GB); Tao YAN, Cambridge (GB); Krystle REISS, San Diego (CA)

(21) Appl. No.: 18/476,911

(22) Filed: Sep. 28, 2023

Related U.S. Application Data
(60) Provisional application No. 63/412,024, filed on Sep. 30, 2022.

(51) Int. Cl. C07D 413/04; C12Q 1/6869

(52) U.S. Cl. CPC C07D 413/04; C12Q 1/6869

(57) ABSTRACT

Embodiments of the present disclosure relate to cyclooctatetraene containing dyes and their uses as fluorescent labels. Also provided are composition containing cyclooctatetraene. The dyes and compositions may be used in various biological applications, such as nucleic acid sequencing.

### FIELD

The present disclosure relates to fluorescent compounds and labeled nucleotides containing one or more photoprotective moieties. In particular, the fluorescent compounds and labeled nucleotides may be used in various biological applications, such as nucleic acid sequencing applications.

### BACKGROUND

### Background

Non-radioactive detection of nucleic acids utilizing fluorescent labels is an important technology in molecular biology. Many procedures employed in recombinant DNA technology previously relied on the use of nucleotides or polynucleotides radioactively labeled with, for example ³²P. Radioactive compounds permit sensitive detection of nucleic acids and other molecules of interest. However, there are serious limitations in the use of radioactive isotopes such as their expense, limited shelf life and more importantly safety considerations Eliminating the need for radioactive labels enhances safety whilst reducing the environmental impact and costs associated with, for example, reagent disposal. Methods amenable to non-radioactive fluorescent detection include by way of non-limiting example, automated DNA sequencing, hybridization methods, real-time detection of polymerase-chain-reaction products and immunoassays.

For many applications it is desirable to employ multiple spectrally distinguishable fluorescent labels in order to achieve independent detection of a plurality of spatially overlapping analytes. In such multiplex methods the number of reaction vessels may be reduced to simplify experimental protocols and facilitate the production of application-specific reagent kits. In multi-color automated DNA sequencing systems for example, multiplex fluorescent detection allows for the analysis of multiple nucleotide bases in a single electrophoresis lane, thereby increasing throughput over single-color methods, and reducing uncertainties associated with inter-lane electrophoretic mobility variations.

However, multiplex fluorescent detection can be problematic and there are a number of important factors that may constrain selection of appropriate fluorescent labels. First, it may be difficult to find dye compounds with suitably-resolved absorption and emission spectra in a given application. In addition, when several fluorescent dyes are used together, generating fluorescence signals in distinguishable spectral regions by simultaneous excitation may be complicated because absorption bands of the dyes are usually widely separated, so it may be difficult to achieve comparable fluorescence excitation efficiencies even for two dyes. Another consideration of particular importance to molecular biology methods is the extent to which the fluorescent dyes must be compatible with reagent chemistries such as, for example, DNA synthesis solvents and reagents, buffers, polymerase enzymes, and ligase enzymes. Further, since many excitation methods use high power light sources like lasers, the fluorescent dyes must be sufficiently photo-stable to withstand multiple excitations.

For high-accuracy fluorescence identification of nucleobases, scanning of fluorescently labeled nucleotides under intensive expose to light is typically involved. Extensive laser irradiation, however, may bleach fluorescent dyes and/or damage nucleotide samples in solution/on flow-cell surface or those to which the fluorescent dyes are conjugated. Such expose to light may also cause DNA sample damage. Thus, there is a need particularly in multiplex fluorescent DNA sequencing to protect fluorescent dyes from photo-bleaching and nucleotides from photo induced damages. The type and extent of photo-bleaching and photo-damage may vary depending on, for example, the compounds' chemical structure and some of their physical-chemical properties like redox potential, excitation spectra of particular bio-label, intensity of particular light source irradiation, and time of exposure in particular measurement. Since lower wavelength light sources are delivering higher energy photons, blue LED/laser having short (400-500 nm) wavelength emission (e.g., 450-460 nm) are more likely to cause photo-bleaching of dyes and associated with light DNA damage.

Performing fluorescent detection steps in an array context, such as sequencing by synthesis, can cause fluorescence signal intensity loss. Oxidative DNA modification may be observed through interaction with different light induced reactive species, for example, reactive oxygen species (ROS) such as singlet oxygen, superoxide anion, and hydroxyl radical. Damage to DNA by ROS causes reduced signal intensity to be observed at later sequencing cycles, significantly impacting signal-noise ratios (SNR) subsequently.

Various compounds may be used to mitigate DNA damages through several mechanisms, such as triplet-triplet annihilation (TTA), triplet state quenching (TSQ), and ROS trapping. Antioxidants, radical scavengers, triplet eliminators, for example, anthracene and vinyl based compounds have attracted attention due to their potential to mitigate photo-bleaching of fluorophores under high irradiance even in the presence of oxygen (See, for example, U.S. Publication No. 2010/0181535A1). However, there are several issues associated with using these compounds in sequencing, including but not limited to the solubility of the compounds, impact on incorporation rate, possibility of π-stacking and dye quenching. Furthermore, such additives and their conjugates were mostly explored for “Green” and “Red” cyanine dyes (See, for example, U.S. Publication No. 2015/0011731A1). As such, there still exists a need for developing a library of high-performance fluorescent bio-labels to be expanded further into the region of lower excitation wavelengths (e.g., 400˜500 nm) where light induced DNA damage is the most pronounced. In addition, there is a need to further reduce fluorescent signal intensity loss for applications in sequencing by synthesis to facilitate sequencing of long nucleotide sequences, including sequences of 50, 75, 100, 200, and 500 nucleotides or more. Described herein are fluorescent compounds and compositions containing various photoprotective moieties that are suitable for nucleotide labeling and nucleic acid sequencing.

### SUMMARY

One aspect of the present disclosure relates to a fluorescent compound excitable by a light source having a wavelength between about 400 nm to about 500 nm, wherein the fluorescent compound is covalently attached to one or more photoprotective moieties comprising the formula (Ia), (Ib), (II), (IIb), (III) or (IV):

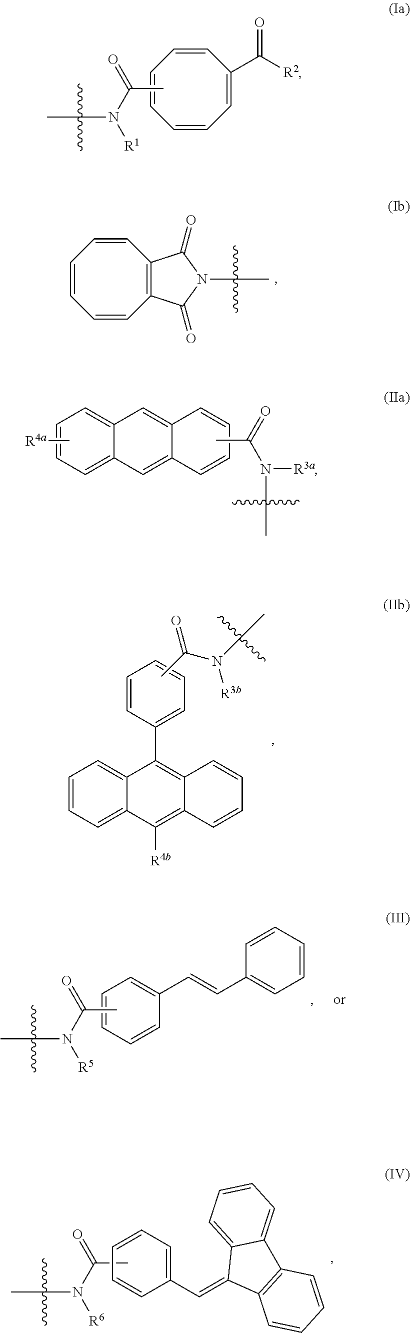

wherein
each of R¹, R^{3a}, R^{3b}, R⁵ and R⁶ is independently H, unsubstituted C₁-C₆ alkyl, or substituted C₁-C₆ alkyl;
R² is —OR⁷, —NR⁸R⁹, —O—, or —NR⁸—;
each of R^{4a} and R^{4b} is independently H or optionally substituted C₆-C₁₀ aryl;
each of R⁷, R⁸ and R⁹ is independently H, unsubstituted C₁-C₆ alkyl, substituted C₁-C₆ alkyl, optionally substituted C₆-C₁₀ aryl, optionally substituted C₃-C₇ cycloalkyl, optionally substituted 5 to 10 membered heteroaryl, or optionally substituted 3 to 10 membered heterocyclyl;
each of the aromatic ring or ring system in formulas (Ia), (Ib), (IIa), (IIb), (III) or (IV) is optionally substituted with one, two or three substituents R^{Ar} independently selected from the group consisting of carboxyl, C₁-C₆ alkyl, substituted C₁-C₆ alkyl, C₁-C₆ alkoxy, substituted C₁-C₆ alkoxy, C₁-C₆ haloalkyl, C₁-C₆ haloalkoxy, (C₁-C₆ alkoxy)C₁-C₆ alkyl, —O(C₁-C₆ alkoxy)C₁-C₆ alkyl, optionally substituted amino, amino(C₁-C₆ alkyl), halo, cyano, hydroxy, hydroxy(C₁-C₆ alkyl), nitro, sulfonyl, sulfo, sulfonate, S-sulfonamido, N-sulfonamido, C₆-C₁₀ aryl, 5 to 10 membered heteroaryl, C₃-C₁₀ carbocyclyl, 3 to 10 membered heterocyclyl, —C(═O)OR¹⁰, —C(═O)NR¹¹-12; —(CH₂CH₂O)n(C₁-C₆ alkyl), and —O(CH₂CH₂O)n(C₁-C₆ alkyl);
each of R¹⁰, R¹¹, and R¹² is independently H, unsubstituted C₁-C₆ alkyl, or substituted C₁-C₆ alkyl; and
n is an integer of 1 to 20. In some embodiment, such fluorescent compound is excitable by a blue light having a wavelength from about 450 nm to about 460 nm.

One aspect of the present disclosure relates to a nucleotide labeled with a fluorescent compound described herein. Oligonucleotides or polynucleotides comprising the labeled nucleotide incorporated thereof are also described herein.

One aspect of the present disclosure relates to a kit comprising a first type of nucleotide, where the first type of nucleotide is the labeled nucleotide with the fluorescent compound as described herein.

Another aspect of the present disclosure relates to a method for determining the sequences of a plurality of different target polynucleotides, comprising:
(a) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(b) contacting the solid support with an aqueous solution comprising DNA polymerase and one more of four different types of nucleotides under conditions suitable for DNA polymerase-mediated primer extension, and incorporating one type of nucleotides into the sequencing primers to produce extended copy polynucleotides, wherein at least one type of nucleotide is a labeled nucleotide described herein, and wherein each of the four types of nucleotides comprises a 3′ blocking group;
(c) imaging the solid support and performing one or more fluorescent measurements of the extended copy polynucleotides; and
(d) removing the 3′ blocking group of the incorporated nucleotides.

A further aspect of the present disclosure relates to a method for reducing light-induced sequencing signal decay during sequencing by synthesis, comprising:
(i) contacting a solid support with an incorporation mixture comprising DNA polymerase and one more of four different types of nucleotides, wherein the solid support comprises a plurality of different target polynucleotides immobilized thereon, and sequencing primers that are complementary and hybridized to at least a portion of the target polynucleotides;
(ii) incorporating one type of nucleotides into the sequencing primers to produce extended copy polynucleotides, wherein one or more four types of nucleotides comprises a detectable label, and each of the four types of nucleotides comprises a 3′ blocking group;
(iii) imaging and performing one or more fluorescent measurements of the extended copy polynucleotides in an aqueous scan mixture to determine the identity of the incorporated nucleotides; and
(iv) removing the 3′ blocking groups and the detectable labels of the incorporated nucleotides;

wherein the aqueous scan mixture comprises one or more additives for reducing fluorescent signal decay caused by the fluorescent measurements, and wherein the one or more additives comprise a tri-substituted 1,4-cyclooctatetraene (COT) analog. In some embodiments, the tri-substituted 1,4-COT analog has the structure of formula (VI):

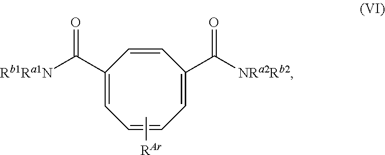

wherein each of R^{a1}, R^{a2}, R^{b1} and R^{b2} is independently H, unsubstituted C₁-C₆ alkyl, or substituted C₁-C₆ alkyl; R^{Ar} is carboxyl, C₁-C₆ alkyl, substituted C₁-C₆ alkyl, C₁-C₆ alkoxy, substituted C₁-C₆ alkoxy, C₁-C₆ haloalkyl, C₁-C₆ halo alkoxy, (C₁-C₆ alkoxy)C₁-C₆ alkyl, —O(C₁-C₆ alkoxy)C₁-C₆ alkyl, optionally substituted amino, amino(C₁-C₆ alkyl), halo, cyano, hydroxy, hydroxy(C₁-C₆ alkyl), nitro, sulfonyl, sulfo, sulfonate, S-sulfonamido, N-sulfonamido, C₆-C₁₀ aryl, 5 to 10 membered heteroaryl, C₃-C₁₀ carbocyclyl, 3 to 10 membered heterocyclyl, —C(═O)OR¹⁰, —C(═O)NR¹¹R¹²; —(CH₂CH₂O)ₙ(C₁C₆ alkyl), and —O(CH₂CH₂O)ₙ(C₁-C₆ alkyl); and each of R¹⁰, R¹¹ and R¹² is independently H, unsubstituted C₁-C₆ alkyl, or substituted C₁-C₆ alkyl.

A further aspect of the present disclosure relates to a kit for use with a sequencing apparatus, comprising a scan mixture composition, wherein the scan mixture composition comprises one or more additives for reducing fluorescent signal decay caused by the fluorescent measurements, and wherein the one or more additives comprise a tri-substituted 1,4-cyclooctatetraene (COT) analog described herein.

### BRIEF DESCRIPTION OF THE DRAWINGS

FIGS. 1A and 1B are scatterplots generated in sequencing by synthesis on Illumina's MiSeq® platform using a fully functionalized nucleotide (ffN) set containing C-sPA-reference dye A with and without the presence of a red dye labeled ffC, respectively.

FIGS. 2A and 2B are scatterplots generated in sequencing by synthesis on Illumina's MiSeq® platform using a fully functionalized nucleotide (ffN) set containing C-sPA-Compound 7B with and without the presence of a red dye labeled ffC, respectively.

FIGS. 3A and 3B are scatterplots generated in sequencing by synthesis on Illumina's MiSeq® platform using a fully functionalized nucleotide (ffN) set containing C-sPA-Compound 6B with and without the presence of a red dye labeled ffC, respectively.

FIG. 4 is a bar chart comparing the % T called intensity after 151 cycles of SBS on Illumina's MiSeq® platform when four different ffCs were used in the incorporation mixture at various light dosage.

FIG. 5A is a scatterplot generated in sequencing by synthesis on Illumina's MiSeq® platform using a fully functionalized nucleotide (ffN) set containing C-sPA-reference dye A.

FIG. 5B is a scatterplot generated in sequencing by synthesis on Illumina's MiSeq® platform using a fully functionalized nucleotide (ffN) set containing C-sPA-Compound 4 with and without the presence of a red dye labeled ffC, respectively.

### DETAILED DESCRIPTION

The present disclosure relates to fluorescent compounds and their application as fluorescent labels for biomolecules. In particular, disclosed herein are fluorescent compounds covalently bonded to one or more photoprotective moieties covalently attached thereto. The photoprotective moiety may comprise a 1,3,5-7-cyclooctatetraene (COT) moiety or a derivative, such as Formula (Ia) or (Ib), an anthracene moiety or a derivative thereof, such as Formula (IIa) or (IIb), a stilbene moiety such as formula (III), or a vinyl substituted fluorene moiety such as formula (IV): as described herein. These fluorescent compounds may be used as labels for nucleic acid sequencing applications, for example, as nucleotide labels during sequencing-by-synthesis. In particular, the fluorescent compounds described herein may be excited by a light source with a wavelength between about 400 nm to about 500 nm, from about 420 nm to about 480 nm, or from about 450 nm to about 460 nm. These photo-protected fluorescent dyes have demonstrated improved signal decay and reduced error rate in sequencing applications.

### Definitions

The section headings used herein are for organizational purposes only and are not to be construed as limiting the subject matter described.

It is noted that, as used in this specification and the appended claims, the singular forms “a”, “an” and “the” include plural referents unless expressly and unequivocally limited to one referent. It will be apparent to those skilled in the art that various modifications and variations can be made to various embodiments described herein without departing from the spirit or scope of the present teachings. Thus, it is intended that the various embodiments described herein cover other modifications and variations within the scope of the appended claims and their equivalents.

Unless defined otherwise, all technical and scientific terms used herein have the same meaning as is commonly understood by one of ordinary skill in the art. The use of the term “including” as well as other forms, such as “include”, “includes,” and “included,” is not limiting. The use of the term “having” as well as other forms, such as “have”, “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps but may include additional steps. When used in the context of a compound, composition, or device, the term “comprising” means that the compound, composition, or device includes at least the recited features or components, but may also include additional features or components.

As used herein, common organic abbreviations are defined as follows:
° C. Temperature in degrees Centigrade
dATP Deoxyadenosine triphosphate
dCTP Deoxycytidine triphosphate
dGTP Deoxyguanosine triphosphate
dTTP Deoxythymidine triphosphate
ddNTP Dideoxynucleotide triphosphate
ffA Fully functionalized A nucleotide
ffC Fully functionalized C nucleotide
ffG Fully functionalized G nucleotide
ffN Fully functionalized nucleotide
ffT Fully functionalized T nucleotide
h Hour(s)
RT Room temperature
SBS Sequencing by Synthesis

As used herein, the term “array” refers to a population of different probe molecules that are attached to one or more substrates such that the different probe molecules can be differentiated from each other according to relative location. An array can include different probe molecules that are each located at a different addressable location on a substrate. Alternatively, or additionally, an array can include separate substrates each bearing a different probe molecule, wherein the different probe molecules can be identified according to the locations of the substrates on a surface to which the substrates are attached or according to the locations of the substrates in a liquid. Exemplary arrays in which separate substrates are located on a surface include, without limitation, those including beads in wells as described, for example, in U.S. Pat. No. 6,355,431 B1, US 2002/0102578 and PCT Publication No. WO 00/63437. Exemplary formats that can be used in the invention to distinguish beads in a liquid array, for example, using a microfluidic device, such as a fluorescent activated cell sorter (FACS), are described, for example, in U.S. Pat. No. 6,524,793. Further examples of arrays that can be used in the invention include, without limitation, those described in U.S. Pat. Nos. 5,429,807; 5,436,327; 5,561,071; 5,583,211; 5,658,734; 5,837,858; 5,874,219; 5,919,523; 6,136,269; 6,287,768; 6,287,776; 6,288,220; 6,297,006; 6,291,193; 6,346,413; 6,416,949; 6,482,591; 6,514,751 and 6,610,482; and WO 93/17126; WO 95/11995; WO 95/35505; EP 742 287; and EP 799 897.

As used herein, the term “covalently attached” or “covalently bonded” refers to the forming of a chemical bonding that is characterized by the sharing of pairs of electrons between atoms. For example, a covalently attached polymer coating refers to a polymer coating that forms chemical bonds with a functionalized surface of a substrate, as compared to attachment to the surface via other means, for example, adhesion or electrostatic interaction. It will be appreciated that polymers that are attached covalently to a surface can also be bonded via means in addition to covalent attachment.

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “Cₐ to C_{b}” in which “a” and “b” are integers refer to the number of carbon atoms in an alkyl, alkenyl or alkynyl group, or the number of ring atoms of a cycloalkyl or aryl group. That is, the alkyl, the alkenyl, the alkynyl, the ring of the cycloalkyl, and ring of the aryl can contain from “a” to “b”, inclusive, carbon atoms. For example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—; a C₃ to C₄ cycloalkyl group refers to all cycloalkyl groups having from 3 to 4 carbon atoms, that is, cyclopropyl and cyclobutyl. Similarly, a “4 to 6 membered heterocyclyl” group refers to all heterocyclyl groups with 4 to 6 total ring atoms, for example, azetidine, oxetane, oxazoline, pyrrolidine, piperidine, piperazine, morpholine, and the like. If no “a” and “b” are designated with regard to an alkyl, alkenyl, alkynyl, cycloalkyl, or aryl group, the broadest range described in these definitions is to be assumed. As used herein, the term “C₁-C₆” includes C₁, C₂, C₃, C₄, C₅ and C₆, and a range defined by any of the two numbers. For example, C₁-C₆ alkyl includes C₁, C₂, C₃, C₄, ₅ and C₆ alkyl, C₂-C₆ alkyl, C₁-C₃ alkyl, etc. Similarly, C₂-C₆ alkenyl includes C₂, C₃, C₄, C₅ and C₆ alkenyl, C₂-05 alkenyl, C₃-C₄ alkenyl, etc.; and C₂-C₆ alkynyl includes C₂, C₃, C₄, C₅ and C₆ alkynyl, C₂-C₅ alkynyl, C₃-C₄ alkynyl, etc. C₃-C₈ cycloalkyl each includes hydrocarbon ring containing 3, 4, 5, 6, 7 and 8 carbon atoms, or a range defined by any of the two numbers, such as C₃-C₇ cycloalkyl or C₅-C₆ cycloalkyl.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 6 carbon atoms. By way of example only, “C₁₋₆ alkyl” or “C₁-C₆ alkyl” indicates that there are one to six carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “alkoxy” refers to the formula —OR wherein R is an alkyl as is defined above, such as “C₁₋₉ alkoxy” or “C₁-C₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 6 carbon atoms. By way of example only, “C₂-C₆alkenyl” or “C₂₋₆ alkenyl” indicates that there are two to six carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, and hexenyl, and the like.

As used herein, “alkynyl” refers to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 6 carbon atoms. By way of example only, “C₂₋₆ alkynyl” or “C₂-C₆alkenyl” indicates that there are two to six carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆-C₁₀ aryl,” “C₆ or C₁₀ aryl,” or similar designations. Examples of aryl groups include, but are not limited to, phenyl, naphthyl, azulenyl, and anthracenyl.

An “aralkyl” or “arylalkyl” is an aryl group connected, as a substituent, via an alkylene group, such as “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁₋₆ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁₋₆ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl”, “C₃-C₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

As used herein, “cycloalkyl” means a fully saturated carbocyclyl ring or ring system. Examples include cyclopropyl, cyclobutyl, cyclopentyl, and cyclohexyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, is oindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

As used herein, “alkoxyalkyl” or “(alkoxy)alkyl” refers to an alkoxy group connected via an alkylene group, such as C₂-C₈ alkoxyalkyl, or (C₁-C₆ alkoxy)C₁-C₆ alkyl, for example, —(CH₂)₁₋₃-OCH₃.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

A “C-carboxy” group refers to a “—C(═O)OR” group in which R is selected from the group consisting of hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein. A non-limiting example includes carboxyl (i.e., —C(═O)OH).

A “sulfonyl” group refers to an “—SO₂R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

A “sulfino” group refers to a “—S(═O)OH” group.

A “sulfo” group refers to a “—S(═O)₂OH” or “—SO₃H” group.

A “sulfonate” group refers to a “—SO₃^{—}” group.

A “sulfate” group refers to “—SO₄⁻” group.

A “S-sulfonamido” group refers to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “N-sulfonamido” group refers to a “—N(R_{A})SO₂R_{B}” group in which R_{A} and R_{b} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

A “C-amido” group refers to a “—C(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “N-amido” group refers to a “—N(R_{A})C(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “amino” group refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein. A non-limiting example includes free amino (i.e., —NH₂).

An “aminoalkyl” group refers to an amino group connected via an alkylene group.

An “alkoxyalkyl” group refers to an alkoxy group connected via an alkylene group, such as a “C₂-C₈alkoxyalkyl” and the like.

When a group is described as “optionally substituted” it may be either unsubstituted or substituted. Likewise, when a group is described as being “substituted”, the substituent may be selected from one or more of the indicated substituents. As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl (C₁-C₆)alkyl (optionally substituted with halo, C₁-C₆ alkyl, C₁-C₆ alkoxy, C₁-C₆ haloalkyl, and C₁-C₆ haloalkoxy), halo, —CN, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, —SO₃H, sulfonate, sulfate, sulfino, —OSO₂C₁-C₄alkyl, and oxo (═O). Wherever a group is described as “optionally substituted” that group can be substituted with the above substituents. In some embodiments, when an alkyl, alkenyl, alkynyl, aryl, heteroaryl, carbocyclyl or heterocyclyl group is substituted, each is independently substituted with one or more substituents selected from the group consisting of halo, —CN, —SO₃⁻, —OSO₃⁻, —SO₃H, —SR^{A}, —OR^{A}, —NR^{B}R^{C}, oxo, —CONR^{B}R^{C}, —SO²NR^{B}R^{C}, —COOH, and —COOR^{B}, where R^{A}, R^{B} and R^{C} are each independently selected from H, alkyl, substituted alkyl, alkenyl, substituted alkenyl, alkynyl, substituted alkynyl, aryl, and substituted aryl.

As understood by one of ordinary skill in the art, a fluorescent compound described herein may exist in ionized form, e.g., the compound may comprise —CO₂⁻, —SO₃ or —O—SO₃⁻. If a compound contains a negatively charged group (such as -SO3), it may also contain a counterion such that the compound as a whole is neutral. The compound may exist in a salt form, where the counterion is provided by a conjugate acid or base. The counterion may not expressly shown in the compound structure.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

When two “adjacent” R groups are said to form a ring “together with the atom to which they are attached,” it is meant that the collective unit of the atoms, intervening bonds, and the two R groups are the recited ring. For example, when the following substructure is present:

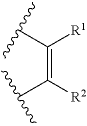

and R¹ and R² are defined as selected from the group consisting of hydrogen and alkyl, or R¹ and R² together with the atoms to which they are attached form an aryl or carbocyclyl, it is meant that R¹ and R² can be selected from hydrogen or alkyl, or alternatively, the substructure has structure:

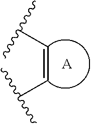

where A is an aryl ring or a carbocyclyl containing the depicted double bond.

Wherever a substituent is depicted as a di-radical (i.e., has two points of attachment to the rest of the molecule), it is to be understood that the substituent can be attached in any directional configuration unless otherwise indicated. Thus, for example, a substituent depicted as —AE— or

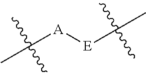

includes the substituent being oriented such that the A is attached at the leftmost attachment point of the molecule as well as the case in which A is attached at the rightmost attachment point of the molecule. In addition, if a group or substituent is depicted as

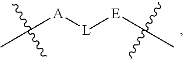

and L is defined an optionally present linker moiety; when L is not present (or absent), such group or substituent is equivalent to

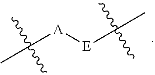

In each instance where a single mesomeric form of a compound described herein is shown, the alternative mesomeric forms are equally contemplated.

As used herein, a “nucleotide” includes a nitrogen containing heterocyclic base, a sugar, and one or more phosphate groups. They are monomeric units of a nucleic acid sequence. In RNA, the sugar is a ribose, and in DNA a deoxyribose, i.e. a sugar lacking a hydroxyl group that is present in ribose. The nitrogen containing heterocyclic base can be purine, deazapurine, or pyrimidine base. Purine bases include adenine (A) and guanine (G), and modified derivatives or analogs thereof, such as 7-deaza adenine or 7-deaza guanine. Pyrimidine bases include cytosine (C), thymine (T), and uracil (U), and modified derivatives or analogs thereof. The C-1 atom of deoxyribose is bonded to N-1 of a pyrimidine or N-9 of a purine.

As used herein, a “nucleoside” is structurally similar to a nucleotide but is missing the phosphate moieties. An example of a nucleoside analogue would be one in which the label is linked to the base and there is no phosphate group attached to the sugar molecule. The term “nucleoside” is used herein in its ordinary sense as understood by those skilled in the art. Examples include, but are not limited to, a ribonucleoside comprising a ribose moiety and a deoxyribonucleoside comprising a deoxyribose moiety. A modified pentose moiety is a pentose moiety in which an oxygen atom has been replaced with a carbon and/or a carbon has been replaced with a sulfur or an oxygen atom. A “nucleoside” is a monomer that can have a substituted base and/or sugar moiety. Additionally, a nucleoside can be incorporated into larger DNA and/or RNA polymers and oligomers.

The term “purine base” is used herein in its ordinary sense as understood by those skilled in the art and includes its tautomers. Similarly, the term “pyrimidine base” is used herein in its ordinary sense as understood by those skilled in the art and includes its tautomers. A non-limiting list of optionally substituted purine-bases includes purine, adenine, guanine, deazapurine, 7-deaza adenine, 7-deaza guanine. hypoxanthine, xanthine, alloxanthine, 7-alkylguanine (e.g., 7-methylguanine), theobromine, caffeine, uric acid and isoguanine. Examples of pyrimidine bases include, but are not limited to, cytosine, thymine, uracil, 5,6-dihydrouracil and 5-alkylcytosine (e.g., 5-methylcytosine).

As used herein, when an oligonucleotide or polynucleotide is described as “comprising” a nucleoside or nucleotide described herein, it means that the nucleoside or nucleotide described herein forms a covalent bond with the oligonucleotide or polynucleotide. Similarly, when a nucleoside or nucleotide is described as part of an oligonucleotide or polynucleotide, such as “incorporated into” an oligonucleotide or polynucleotide, it means that the nucleoside or nucleotide described herein forms a covalent bond with the oligonucleotide or polynucleotide. In some such embodiments, the covalent bond is formed between a 3′ hydroxy group of the oligonucleotide or polynucleotide with the 5′ phosphate group of a nucleotide described herein as a phosphodiester bond between the 3′ carbon atom of the oligonucleotide or polynucleotide and the 5′ carbon atom of the nucleotide.

As used herein, the term “cleavable linker” is not meant to imply that the whole linker is required to be removed. The cleavage site can be located at a position on the linker that ensures that part of the linker remains attached to the detectable label and/or nucleoside or nucleotide moiety after cleavage.

As used herein, “derivative” or “analog” means a synthetic nucleotide or nucleoside derivative having modified base moieties and/or modified sugar moieties. Such derivatives and analogs are discussed in, e.g., Scheit, Nucleotide Analogs (John Wiley & Son, 1980) and Uhlman et al., Chemical Reviews 90:543-584, 1990. Nucleotide analogs can also comprise modified phosphodiester linkages, including phosphorothioate, phosphorodithioate, alkyl-phosphonate, phosphoranilidate and phosphoramidate linkages. “Derivative”, “analog” and “modified” as used herein, may be used interchangeably, and are encompassed by the terms “nucleotide” and “nucleoside” defined herein.

As used herein, the term “phosphate” is used in its ordinary sense as understood by those skilled in the art, and includes its protonated forms (for example,

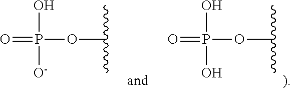 [SMILES: *OP(=O)([O-])O ; *OP(=O)(O)O]

As used herein, the terms “monophosphate,” “diphosphate,” and “triphosphate” are used in their ordinary sense as understood by those skilled in the art, and include protonated forms.

The terms “protecting group” and “protecting groups” as used herein refer to any atom or group of atoms that is added to a molecule in order to prevent existing groups in the molecule from undergoing unwanted chemical reactions. Sometimes, “protecting group” and “blocking group” can be used interchangeably.

As used herein, the prefixes “photo” or “photo-” mean relating to light or electromagnetic radiation. The term can encompass all or part of the electromagnetic spectrum including, but not limited to, one or more of the ranges commonly known as the radio, microwave, infrared, visible, ultraviolet, X-ray or gamma ray parts of the spectrum. The part of the spectrum can be one that is blocked by a metal region of a surface such as those metals set forth herein. Alternatively, or additionally, the part of the spectrum can be one that passes through an interstitial region of a surface such as a region made of glass, plastic, silica, or other material set forth herein. In particular embodiments, radiation can be used that is capable of passing through a metal. Alternatively, or additionally, radiation can be used that is masked by glass, plastic, silica, or other material set forth herein.

As used herein, the term “phasing” refers to a phenomenon in SBS that is caused by incomplete removal of the 3′ blocking groups and fluorescent labels, and failure to complete the incorporation of a portion of DNA strands within clusters by polymerases at a given sequencing cycle. Pre-phasing is caused by the incorporation of nucleotides without effective 3′ blocking groups, wherein the incorporation event goes 1 cycle ahead due to a termination failure. Phasing and pre-phasing cause the measured signal intensities for a specific cycle to consist of the signal from the current cycle as well as noise from the preceding and following cycles. As the number of cycles increases, the fraction of sequences per cluster affected by phasing and pre-phasing increases, hampering the identification of the correct base. Pre-phasing can be caused by the presence of a trace amount of unblocked 3′-OH nucleotides during sequencing by synthesis (SBS). The unblocked 3′-OH nucleotides could be generated during the manufacturing processes or possibly during the storage and reagent handling processes.

As used herein, the term “aqueous scan mixture” refers to an aqueous solution used during an imaging and/or detecting step of sequencing which employs light irradiation to identify the nucleotides incorporated to a plurality of different polynucleotide strands that are complementary to template DNAs. An aqueous scan mixture typically contains one or more antioxidants, or reagents (such as phenolic compounds) that can act as scavengers or quenchers to oxygen radicals or other radicals formed during the imaging/detecting step, for example, those described in U.S. Ser. No. 18/192288, which is incorporated by reference in its entirety. In some embodiments, the aqueous scan mixture may contain a quaternary ammonium surfactant such as cetyltrimethylammonium bromide (CTAB). In some embodiments, the aqueous scan mixture may contain a tri-substituted 1,4-COT analog as described herein.

As used herein, the term “light-induced degradation” means the light-induced damage to one or more nucleic acids or polynucleotide strands in an array of nucleic acids by exposure to light illumination. Such degradation includes the complete or partial removal of individual nucleic acids from the support to which the array is attached. For example, light-induced degradation can include cleavage of the phosphodiester backbone at any of the nucleotides within an individual nucleic acid. Such degradation can also include removal of or reaction of a nucleic acid base or fluorescent tag causing a loss in hybridization or fluorescence function. Light-induced degradation can also include photo-induced crosslinking of nucleotides. The result of light-induced degradation can manifest as a decrease in fluorescence detection sensitivity in one or more regions or sub-arrays of an array nucleic acids when cycling through repeated detection steps, as might be observed, for example, when performing sequencing by synthesis, sequencing by ligation and microarray scanning. When used in conjunction with the term “inhibiting,” this refers to a complete or partial block in the extent of damage, for example, as can be quantified by the observed strength of fluorescent emission. Light induced damage can be presented, for example, as a function of fluorescence signal intensity decay versus number of repeated irradiation (detection) steps performed on the array of nucleic acids. This process is sometimes referred to as signal intensity decay. Another assessment of light damage can be estimated as a function of sequencing error rate versus number of repeated irradiation (detection) steps performed on the array of nucleic acids.

### Fluorescent Compounds with Photoprotective Moieties

Some embodiments of the present application relate to a fluorescent compound excitable by a light source having a wavelength between about 400 nm to about 500 nm, wherein the fluorescent compound is covalently attached to one or more photoprotective moieties comprising the formula (Ia), (Ib), (IIa), (IIb), (III) or (IV):

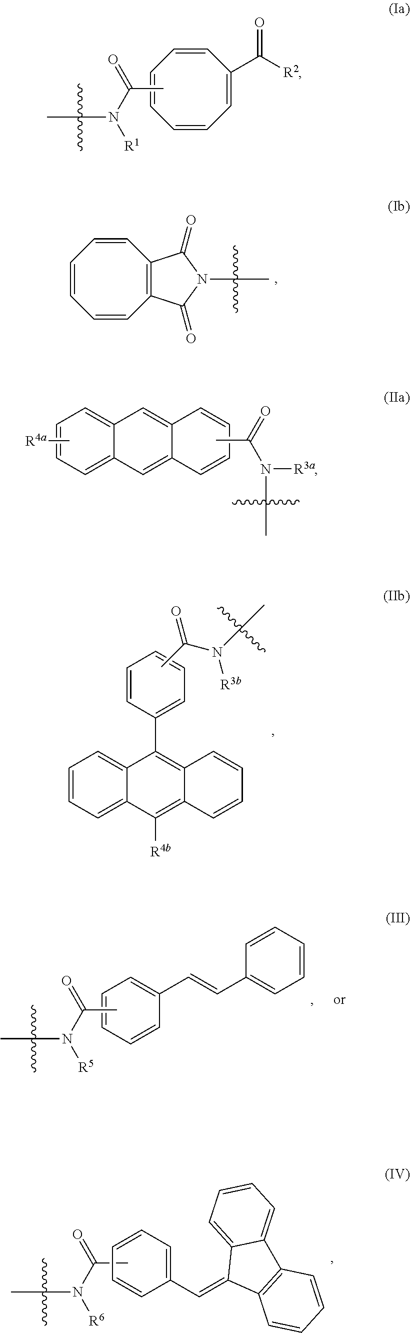

wherein
each of R¹, R^{3a}, R^{3b}, R⁵ and R⁶ is independently H, unsubstituted C₁-C₆ alkyl, or substituted C₁-C₆ alkyl;
R² is —OR⁷, —NR⁸ R⁹, —O—, or —NR⁸—;
each of R^{4a} and R^{4b} is independently H or optionally substituted C₆-C₁₀ aryl;
each of R⁷, R⁸, R⁹ is independently H, unsubstituted C₁-C₆ alkyl, substituted C₁-C₆ alkyl, optionally substituted C₆-C₁₀ aryl, optionally substituted C₃-C₇ cycloalkyl, optionally substituted 5 to 10 membered heteroaryl, or optionally substituted 3 to 10 membered heterocyclyl;

each of the aromatic ring or ring system in formulas (Ia), (Ib), (IIa), (IIb), (III) or (IV) is optionally substituted with one, two or three R^{Ar} independently selected from the group consisting of carboxyl, C₁-C₆ alkyl, substituted C₁-C₆ alkyl, C₁-C₆ alkoxy, substituted C₁-C₆ alkoxy, C₁-C₆ haloalkyl, C₁-C₆ haloalkoxy, (C₁-C₆ alkoxy)C₁-C₆ alkyl, —O(C₁-C₆ alkoxy)C₁-C₆ alkyl, optionally substituted amino, amino(C₁-C₆ alkyl), halo, cyano, hydroxy, hydroxy(C₁-C₆ alkyl), nitro, sulfonyl, sulfo, sulfonate, S-sulfonamido, N-sulfonamido, C₆-C₁₀ aryl, 5 to 10 membered heteroaryl, C₃-C₁₀ carbocyclyl, 3 to 10 membered heterocyclyl, —C(═O)OR¹⁰, —C(═O)NR¹¹ R¹²; —(CH₂CH₂O)ₙ(C₁-C₆ alkyl), and —O(CH₂CH₂O)ₙ(C₁-C₆ alkyl);

each of R¹⁰, R¹¹, and R¹² is independently H, unsubstituted C₁-C₆ alkyl, or substituted C₁-C₆ alkyl; and

n is an integer of 1 to 20.

In some embodiments, the one or more photoprotective moieties of formula (Ia) is also represented by formula (Ia-1) or (Ia-2):

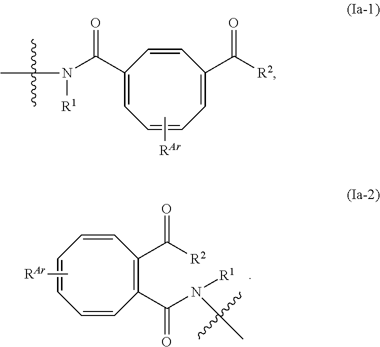

In some further embodiments, the photoprotective moieties of formula (Ia-1) or (Ia-2) may also be represented by formula (Ia-3) or (Ia-4):

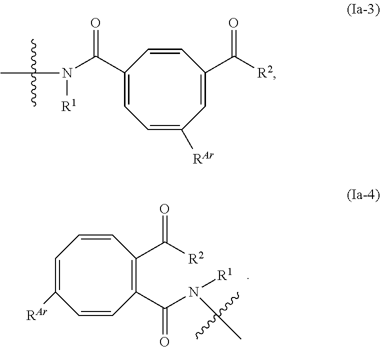

In some such embodiments, R¹ is H or substituted C₁-C₆ alkyl (for example, substituted methyl, ethyl, n-propyl, isopropyl, n-butyl, t-butyl, n-pentyl, isopentyl or n-hexyl). In some embodiments, R² is —OH, —NR⁸ R⁹, or —NR⁸—. In some embodiments, each of R⁸ is independently H or substituted C₁-C₆ alkyl. In some embodiments, R⁹ is unsubstituted or substituted C₁-C₆ alkyl. In some embodiments, each R^{Ar} is independently carboxyl, sulfo, sulfonate, —O(CH₂CH₂O)ₙCH₃, —C(═O)NH(CH₂)₂₋₅SO₃H, chloro, bromo, nitro, phenyl, C₁-C₆ alkyl (such as methyl, ethyl, isopropyl, propyl, n-butyl, t-butyl), —NH(C₁-C₆ alkyl) (e.g., —NHCH₃ or NHCH₂CH₃), or —N(C₁-C₆alkyl)₂ (e.g., —N(CH₃)₂, —N(CH₃)(CH₂CH₃) or —N(CH₂CH₃)₂).

In some embodiments, the one or more photoprotective moieties of formula (IIa) is also represented by formula (IIa-1) or (IIa-2):

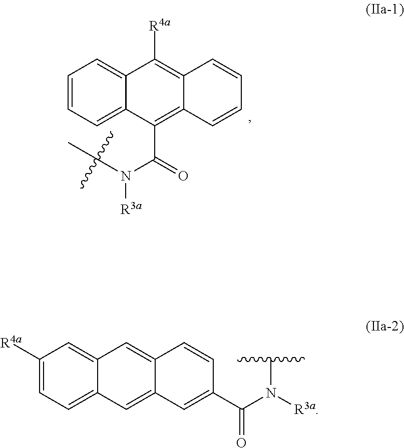

In some such embodiments, R^{3a} is H or substituted C₁-C₆ alkyl (for example, substituted methyl, ethyl, n-propyl, isopropyl, n-butyl, t-butyl, n-pentyl, isopentyl or n-hexyl). In some such embodiments, R^{4a} is H, unsubstituted phenyl, or substituted phenyl. In some further embodiments, the aromatic ring of the photoprotective moieties of formula (IIa), (IIa-1) or (IIa-2) is independently optionally substituted with one R^{Ar}. In further embodiments, each R^{Ar} is independently carboxyl, sulfo, sulfonate, —O(CH₂CH₂O)ₙCH₃, —C(═O)NH(CH₂)₂₋₅SO₃H, chloro, bromo, nitro, phenyl, C₁-C₆ alkyl (such as methyl, ethyl, isopropyl, propyl, n-butyl, t-butyl), —NH(C₁-C₆ alkyl) (e.g., —NHCH₃ or NHCH₂CH₃), or —N(C₁-C₆ alkyl)₂ (e.g., —N(CH₃)₂, —N(CH₃)(CH₂CH₃) or —N(CH₂CH₃)₂).

In some embodiments, the one or more photoprotective moieties of formula (IIb) is also represented by formula (IIb-1):

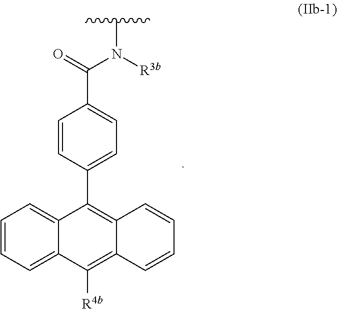

In some such embodiments, R^{3b} is H or substituted C₁-C₆ alkyl (for example, substituted methyl, ethyl, n-propyl, isopropyl, n-butyl, t-butyl, n-pentyl, isopentyl or n-hexyl). In some such embodiments, R^{4b} is H, unsubstituted phenyl, or substituted phenyl. In some further embodiments, the aromatic ring of the photoprotective moieties of formula (IIb) or (IIb-1) is independently optionally substituted with one R^{Ar}. In further embodiments, each R^{Ar} is independently carboxyl, sulfo, sulfonate, —O(CH₂CH₂O)ₙCH₃, —C(═O)NH(CH₂)₂₋₅SO₃H, chloro, bromo, nitro, phenyl, C₁-C₆ alkyl (such as methyl, ethyl, isopropyl, propyl, n-butyl, t-butyl), —NH(C₁-C₆ alkyl) (e.g., —NHCH₃ or NHCH₂CH₃), or —N(C₁-C₆ alkyl)₂ (e.g., —N(CH₃)₂, —N(CH₃)(CH₂CH₃) or —N(CH₂CH₃)₂).

In some embodiments, the one or more photoprotective moieties of formula (III) is also represented by formula (III-1):

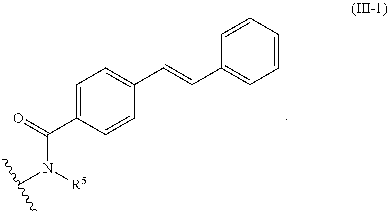

In some such embodiments, R⁵ is H or substituted C₁-C₆ alkyl (for example, substituted methyl, ethyl, n-propyl, isopropyl, n-butyl, t-butyl, n-pentyl, isopentyl or n-hexyl). In some further embodiments, the aromatic ring of the photoprotective moieties of formula (III) or (III-1) is independently optionally substituted with one R^{Ar}. In further embodiments, each R^{Ar} is independently carboxyl, sulfo, sulfonate, −O(CH₂CH₂O)ₙCH₃, —C(═O)NH(CH₂)₂₋₅SO₃H, chloro, bromo, nitro, phenyl, C₁-C₆ alkyl (such as methyl, ethyl, isopropyl, propyl, n-butyl, t-butyl), —NH(C₁-C₆ alkyl) (e.g., —NHCH₃ or NHCH₂CH₃), or —N(C₁-C₆ alkyl)₂ (e.g., —N(CH₃)₂, —N(CH₃)(CH₂CH₃) or —N(CH₂CH₃)₂).

In some embodiments, the one or more photoprotective moieties of formula (IV) is also represented by formula (IV-1) or (IV-2):

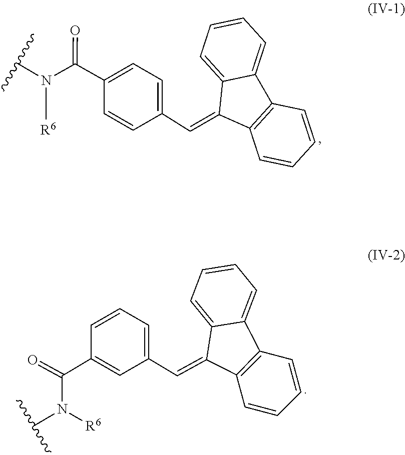

In some such embodiments, R⁶ is H or substituted C₁-C₆ alkyl) for example, substituted methyl, ethyl, n-propyl, isopropyl, n-butyl, t-butyl, n-pentyl, isopentyl or n-hexyl). In some further embodiments, the aromatic ring of the photoprotective moieties of formula (IV), (IV-1) or (IV-2) is independently optionally substituted with one R^{Ar}. In further embodiments, each R^{Ar} is independently carboxyl, sulfo, sulfonate, —O(CH₂CH₂O)ₙCH₃, —C(═O)NH(CH₂)₂₋₅SO₃H, chloro, bromo, nitro, phenyl, C₁-C₆ alkyl (such as methyl, ethyl, isopropyl, propyl, n-butyl, t-butyl), —NH(C₁-C₆ alkyl) (e.g., —NHCH₃ or NHCH₂CH₃), or —N(C₁-C₆ alkyl)₂ (e.g., —N(CH₃)₂, —N(CH₃)(CH₂CH₃) or —N(CH₂CH₃)₂).

In any embodiments of the photoprotective moieties described herein, the substituted C₁-C₆ alkyl can be substituted with one or more of substituents selected from the group consisting of amino, carboxyl (—COOH), carboxylate, sulfo (—SO₃H), sulfonate (—SO₃⁻), and —C(═O)NR¹¹ R¹², wherein each of R¹¹ and R¹² is independently H, unsubstituted C₁-C₆ alkyl, or C₁-C₆ alkyl substituted with carboxyl, sulfo or sulfonate. In some embodiments, the C₁-C₆ alkyl can be substituted with —C(═O)NR¹¹ R¹², wherein R¹¹ is H or a substituted C₁-C₆ alky (for example, C₂, C₃, C₄ or C₅ alkyl substituted with carboxyl, sulfo, or sulfonate), and R¹² is a substituted C₁-C₆ alky (for example, C₂, C₃, C₄ or C₅ alkyl substituted with carboxyl, sulfo, or sulfonate). In further embodiments, the C₁-C₆ alkyl can be substituted with carboxyl, sulfo, sulfonate, or —C(═O)NH(CH₂)₂₋₅SO₃H.

In some embodiments, each of the aromatic ring or ring system in formulas (Ia-1), (Ia-2), (Ia-3), (Ia-4), (Ib), (IIa), (IIa-1), (IIa-2), (IIb), (IIb-1), (III), (III-1), (IV), (IV-1) or (IV-2) as described herein is not substituted. In other embodiments, such aromatic ring or ring system can be substituted with one, two or three R^{Ar} substituents independently selected from the group consisting of carboxyl, C₁-C₆ alkyl, substituted C₁-C₆ alkyl, C₁-C₆ alkoxy, substituted C₁-C₆ alkoxy, C₁-C₆ halo alkyl, C₁-C₆ halo alkoxy, (C₁-C₆ alkoxy)C₁-C₆ alkyl, —O(C₁-C₆ alkoxy)C₁-C₆ alkyl, optionally substituted amino, amino(C₁-C₆ alkyl), halo, cyano, hydroxy, hydroxy(C₁-C₆ alkyl), nitro, sulfonyl, sulfo, sulfonate, S-sulfonamido, N-sulfonamido, C₆-C₁₀ aryl, 5 to 10 membered heteroaryl, C₃-C₁₀ carbocyclyl, 3 to 10 membered heterocyclyl, —C(═O)OR¹⁰, —C(═O)NR¹¹R¹², —(CH₂CH₂O)ₙ(C₁-C₆ alkyl), and —O(CH₂CH₂O)ₙ(C₁-C₆ alkyl). In further embodiments, the aromatic ring or ring system can be substituted with one or more substituents for improving the water/aqueous solubility of the fluorescent compound, such as sulfo, sulfonate, —C(═O)NR¹¹R¹², (wherein R¹¹ is H or a substituted C₁-C₆ alky (for example, C₂, C₃, C₄ or C₅ alkyl substituted with carboxyl, sulfo, or sulfonate), and R¹² is a substituted C₁-C₆ alky (for example, C₂, C₃, C₄ or C₅ alkyl substituted with carboxyl, sulfo, or sulfonate). In further embodiments, the aromatic ring or ring system can be substituted with carboxyl, sulfo, sulfonate, —O(CH₂CH₂O)ₙCH₃, or —C(═O)NH(CH₂)₂₋₅SO₃H. In other embodiments, the aromatic ring or ring system can be substituted with one R^{Ar} selected from chloro, bromo, nitro, phenyl, C₁-C₆ alkyl (such as methyl, ethyl, isopropyl, propyl, n-butyl, t-butyl), —NH(C₁-C₆ alkyl) (e.g., —NHCH₃ or NHCH₂CH₃), or —N(C₁-C₆alkyl)₂ (e.g., —N(CH₃)₂, —N(CH₃)(CH₂CH₃) or —N(CH₂CH₃)₂).

It is understood that when R² is —O— or —NR⁸—, the photoprotective moiety of formula (Ia) is a divalent moiety.

In some embodiments, the fluorescent compound described herein may be excited by a light source having a wavelength from about 420 nm to about 480 nm, or from about 450 nm to about 460 nm. In some such embodiments, the fluorescent compound is excitable by a blue light and such compound is also referred to as a “blue dye.”

In some embodiments, the fluorescent compound described herein is covalently attached to the one or more photoprotective moieties optionally through a linker of formula (V):

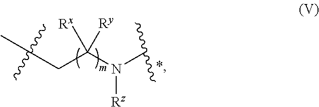

wherein
each R^{x} and R^{y} is independently H, carboxyl, carboxylate, amino, sulfo, sulfonate, —C(O)OR^{a}, or —C(O)NR^{b}R^{c}, or C₁-C₆ alkyl substituted with amino, carboxyl, carboxylate, sulfo, sulfonate, —C(O)OR^{a}, or —C(O)NR^{b}R^{c},

R^{z} is H or C₁-C₆ alkyl substituted with amino, carboxyl, carboxylate, sulfo, sulfonate, —C(O)OR^{a}, or —C(O)NR^{b}R^{c};
R^{a} is optionally substituted C₁-C₆ alkyl, optionally substituted C₆-C₁₀ aryl, optionally substituted 5 to 10 membered heteroaryl, or optionally substituted C₃-C₇ cycloalkyl;
each of R b and RC is independently H, optionally substituted C₁-C₆ alkyl, optionally substituted C₆-C₁₀ aryl, optionally substituted 5 to 10 membered heteroaryl, or optionally substituted C₃-C₇ cycloalkyl;
the carbon atom to which R^{x} and R^{y} are attached in

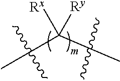

is optionally replaced with O, S, or N, provided that when said carbon atom is replaced with O or S, then R^{x} and R^{y} are both absent; when said carbon atom is replaced with N, then R^{y} is absent; and
m is an integer between 0 to 10 or 1 and 10;
wherein the asterisk * indicates the attachment point of the linker to the fluorescent compound.

In some embodiments of the linker of formula (V), R^{z} is H. In other embodiments, R^{z} is C₁-C₆ alkyl substituted with carboxyl, sulfo, or sulfonate. In some embodiments, m is 1, 2, 3 or 4. In some embodiments, each of R^{x} and R^{y} is H. In one embodiment, m is 1, R^{x} is H and R^{y} is carboxyl. In some other embodiments, when m is greater than 1, R^{x} is H, and each R^{y} is independently H, amino, or carboxyl, wherein at least one R^{y} is carboxyl. For example, when m is 2, one of R^{y} is H and the other R^{y} (attached to same carbon atom that —NR^{z} is also attached to) is carboxyl. When m is 3, two of R^{y} is H and the last R^{y} (attached to same carbon atom that —NR^{z} is also attached to) is carboxyl. When m is 4, three of R^{y} is H and the last R^{y} (attached to same carbon atom that —NR^{z} is also attached to) is carboxyl. Further non-limiting examples of

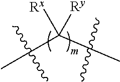

includes the following structures:

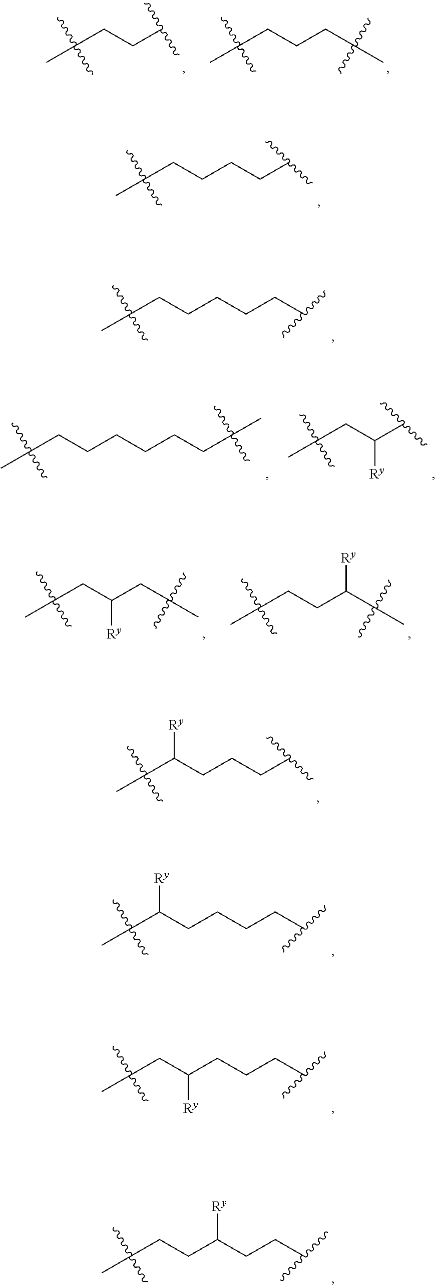

where each R^{y} is independently amino, carboxyl, —C(O)OR^{a}, or —C(O)NR^{b}R^{c} as described here. In further embodiments, any of the CH₂ carbon atom may also be replaced with O, S, or NH.

Non-limiting examples of the photoprotective moiety or moieties described herein are illustrated below:

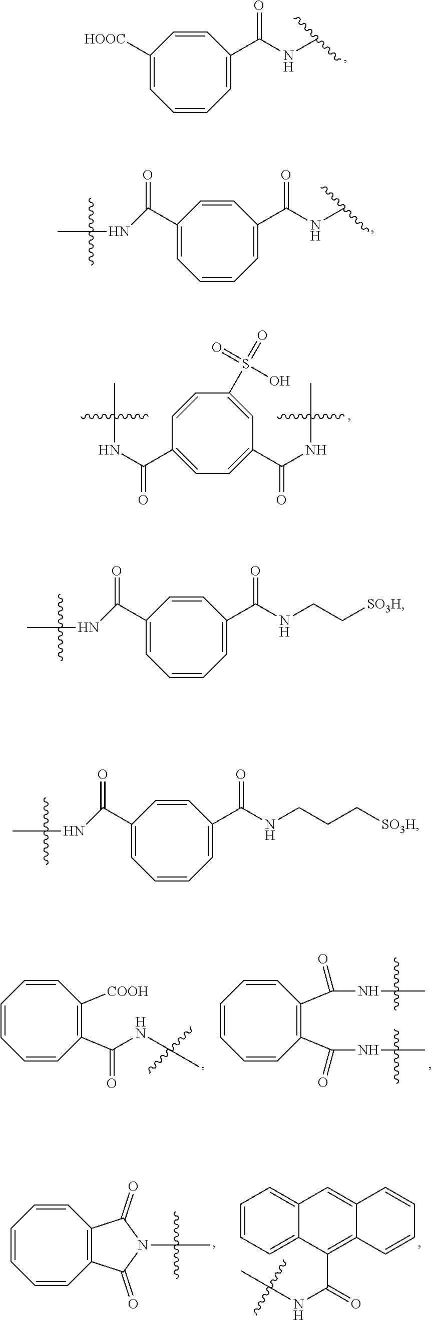

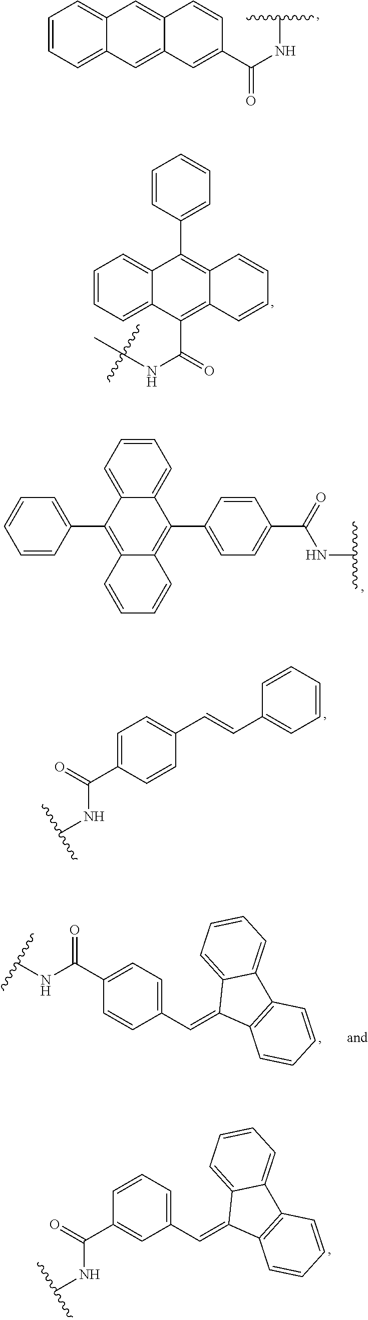 [SMILES: *NC(=O)c1ccc2cc3ccccc3cc2c1 ; *NC(=O)c1c2ccccc2c(-c2ccccc2)c2ccccc12 ; *NC(=O)c1ccc(-c2c3ccccc3c(-c3ccccc3)c3ccccc23)cc1 ; *NC(=O)c1ccc(/C=C/c2ccccc2)cc1 ; *NC(=O)c1ccc(C=C2c3ccccc3-c3ccccc32)cc1 ; *NC(=O)c1cccc(C=C2c3ccccc3-c3ccccc32)c1]

where the aromatic ring or ring system of the photoprotective moieties is either unsubstituted, or substituted with one, two or three R^{Ar} substituents described herein. In further embodiments, the aromatic ring or ring system can be substituted with one or more R^{Ar} such as carboxyl, sulfo, sulfonate, —O(CH₂CH₂O)ₙCH₃, or —C(═O)NH(CH₂)₂₋₅SO₃H. In other embodiments, R^{Ar} can be chloro, bromo, nitro, phenyl, C₁-C₆ alkyl (such as methyl, ethyl, isopropyl, propyl, n-butyl, t-butyl), —NH(C₁-C₆ alkyl) (e.g., —NHCH₃ or NHCH₂CH₃), or —N(C₁-C₆ alkyl)₂ (e.g., —N(CH₃)₂, —N(CH₃)(CH₂CH₃) or —N(CH₂CH₃)₂). In further embodiments, the hydrogen in the —NH— or —C(═O)NH— portion of the photoprotective moiety may be replaced with a C₁-C₆ alkyl substituted with carboxyl, sulfo or sulfonate. When a moiety contains a sulfo group (—SO₃H), it may also be present in the deprotonated form (—SO₃⁻).

Non-limiting examples of the photoprotective moiety or moieties described herein

with a covalently attached linker of formula (V) are illustrated below:

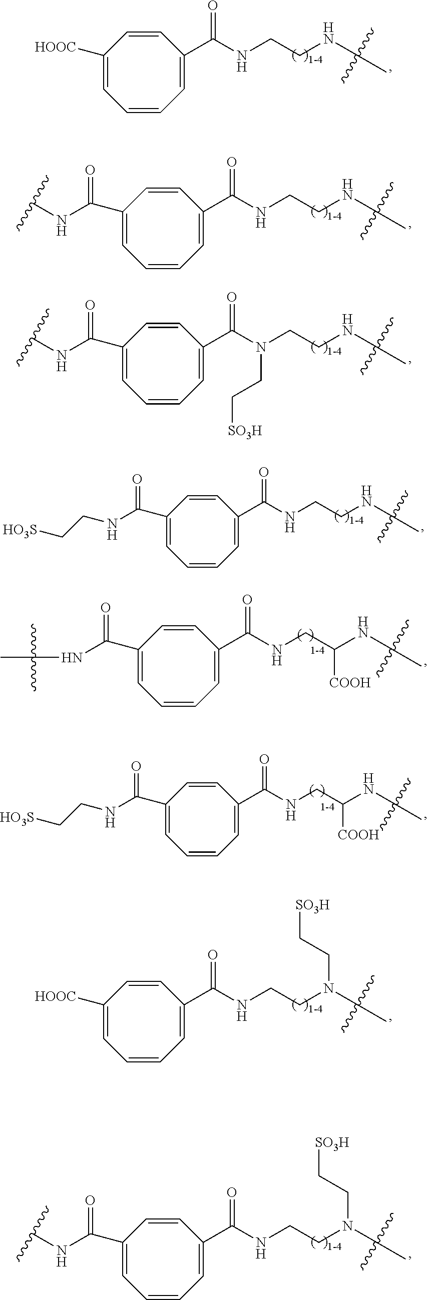

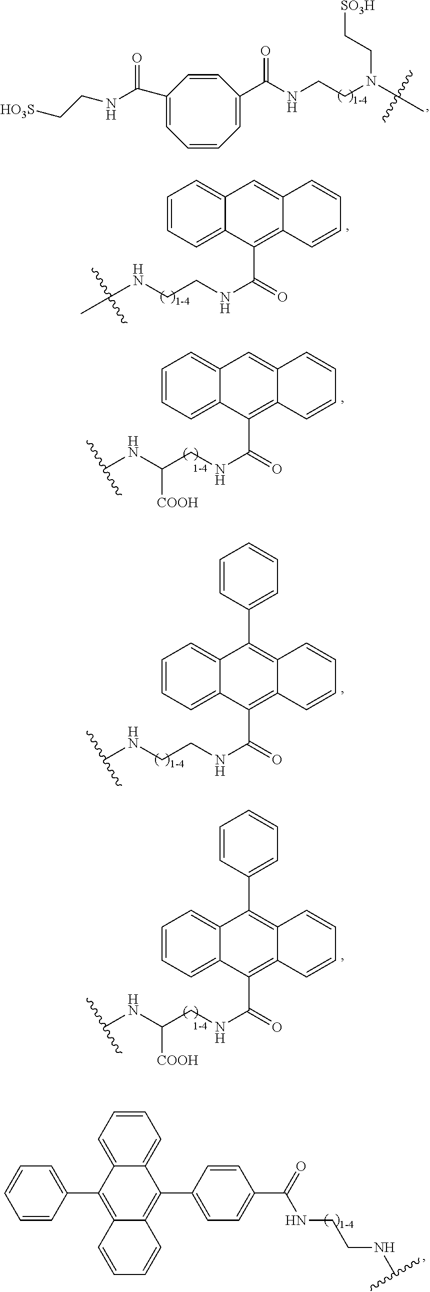

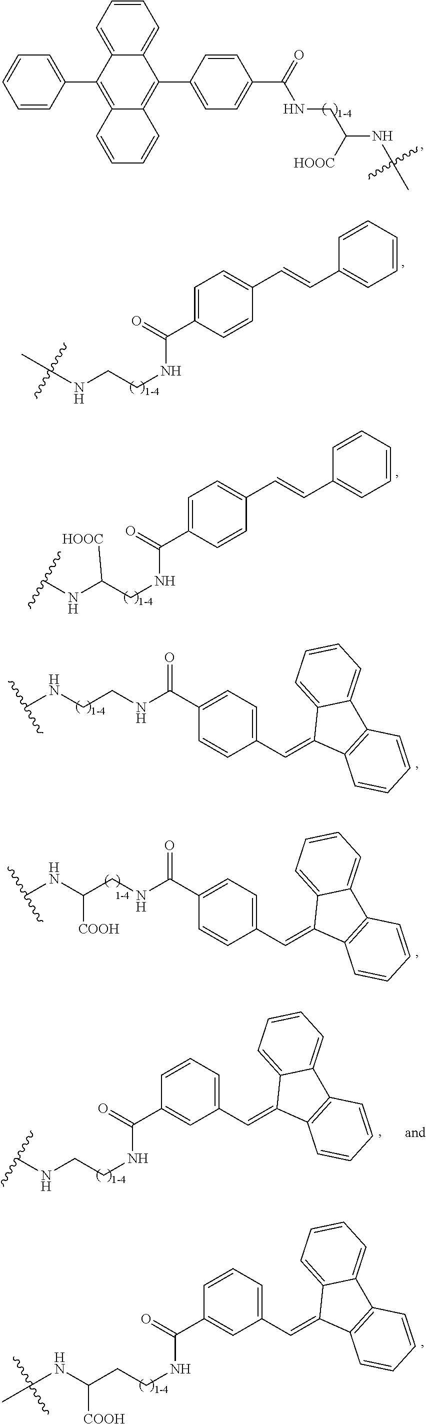

where the aromatic ring or ring system of the photoprotective moieties is either unsubstituted, or substituted with one, two or three R^{Ar} substituents described herein. In further embodiments, the aromatic ring or ring system can be substituted with one or more R^{Ar} such as carboxyl, sulfo, sulfonate, —O(CH₂CH₂O)nCH₃, —C(═O)NH(CH₂)₂₋₅SO₃H, chloro, bromo, nitro, phenyl, C₁-C₆ alkyl (such as methyl, ethyl, isopropyl, propyl, n-butyl, t-butyl), —NH(C₁-C₆ alkyl) (e.g., —NHCH₃ or NHCH₂CH₃), or —N(C₁-C₆ alkyl)₂ (e.g., —N(CH₃)₂, —N(CH₃)(CH₂CH₃) or —N(CH₂CH₃)₂). In further embodiments, the hydrogen in one or more —NH— or —C(═O)NH— portion of the photoprotective moiety may be replaced with a C₁-C₆ alkyl substituted with carboxyl, sulfo or sulfonate. When a moiety contains a sulfo group (—SO₃H), it may also be present in the deprotonated form (—SO₃⁻).

In some embodiments of the linker of formula (V), the asterisk * in formula (V) is connected to a carbonyl group (—C(═O)—) of an amide bond formed by the reaction of an amino group of the photo-protecting moiety with a carboxyl group of the fluorescent compound. In some embodiments, the fluorescent compound having the moiety:

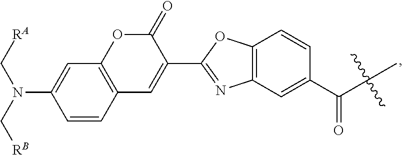

wherein each of R^{A} and R^{B} is independently methyl or substituted C₁-C₆ alkyl (for example, C₂, C₃, C₄ or C₅ alkyl), including C₁-C₆ alkyl substituted with halo, hydroxy, (C₁-C₆ alkyl) amino, (C₁-C₆ alkyl)₂ amino (e.g. —N(Me)₂), sulfo, sulfonate, —C(O)OC₁-C₆ alkyl, optionally substituted 5 to 10 membered heteroaryl/heteroaryloxy or 3 to 10 membered heterocyclyl/heterocyclyloxy, each independently containing one, two or three heteroatoms selected from O, S and N (e.g., furanyl, thienyl, pyrrolyl, pyridyl, imidazolyl, thiazolyl, pyrimidyl, pyrrolidyl, piperidyl, morpholinyl, etc.), optionally substituted C₆-C₁₀ aryl (e.g., phenyl substituted with hydroxy), optionally substituted C₆-C₁₀ aryloxy (e.g., phenoxy substituted with hydroxy), optionally substituted C₃ to C₁₀ carbocyclyl, optionally substituted C₃ to C₁₀ carbocyclyloxy, or one or more photoprotective moieties of the formula (Ia), (Ia-1), (Ia-2), (Ib), (IIa), (IIa-1), (IIa-2), (IIb), (IIb-1), (III), (III-1), (IV), (IV-1) or (IV-2) as described herein. Alternative, R^{A} and R^{B} together with the atoms to which they are attached, form a 4, 5 or 6 membered heterocyclyl containing one nitrogen atom, and the 4, 5 or 6 membered heterocyclyl is optionally substituted with the same substituents as described herein. In some embodiments, the fluorescent compound is referred to as Reference dye A or dye B, having the structures:

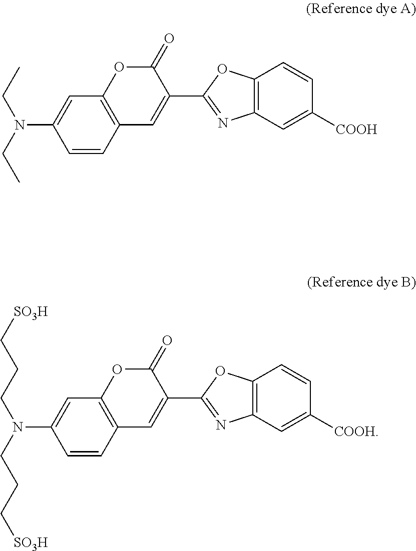 [SMILES: CCN(CC)c1ccc2cc(-c3nc4cc(C(=O)O)ccc4o3)c(=O)oc2c1 ; O=C(O)c1ccc2oc(-c3cc4ccc(N(CCCS(=O)(=O)O)CCCS(=O)(=O)O)cc4oc3=O)nc2c1]

Non-limiting examples of Reference dye A or dye B with one or more photoprotective moieties described herein are illustrated below, as well as salts and mesomeric forms thereof.

| Compound # | Structure |
|---|---|
| Compound 1 | 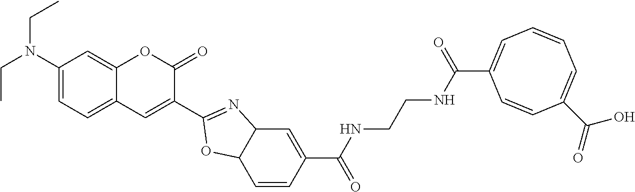 [SMILES: CCN(CC)c1ccc2cc(C3=NC4C=C(C(=O)NCCNC(=O)C5=C/C=C\C=C(C(=O)O)/C=C\5)C=CC4O3)c(=O)oc2c1] |
| Compound 2 | 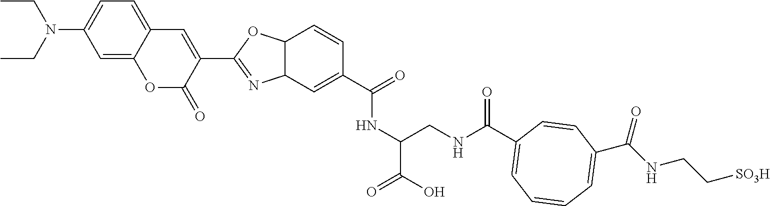 [SMILES: CCN(CC)c1ccc2cc(C3=NC4C=C(C(=O)NC(CNC(=O)C5=C/C=C\C=C(C(=O)NCCS(=O)(=O)O)/C=C\5)C(=O)O)C=CC4O3)c(=O)oc2c1] |
| Compound 3 | 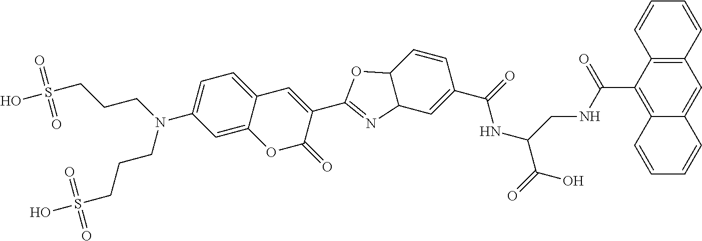 [SMILES: O=C(NC(CNC(=O)c1c2ccccc2cc2ccccc12)C(=O)O)C1=CC2N=C(c3cc4ccc(N(CCCS(=O)(=O)O)CCCS(=O)(=O)O)cc4oc3=O)OC2C=C1] |
| Compound 4 | 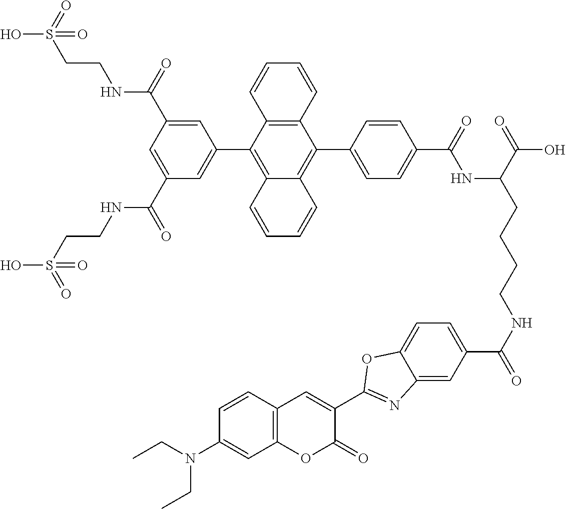 [SMILES: CCN(CC)c1ccc2cc(-c3nc4cc(C(=O)NCCCCC(NC(=O)c5ccc(-c6c7ccccc7c(-c7cc(C(=O)NCCS(=O)(=O)O)cc(C(=O)NCCS(=O)(=O)O)c7)c7ccccc67)cc5)C(=O)O)ccc4o3)c(=O)oc2c1] |
| Compound 5A | 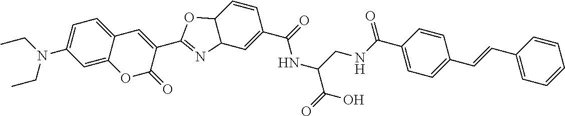 [SMILES: CCN(CC)c1ccc2cc(C3=NC4C=C(C(=O)NC(CNC(=O)c5ccc(/C=C/c6ccccc6)cc5)C(=O)O)C=CC4O3)c(=O)oc2c1] |
| Compound 5B | 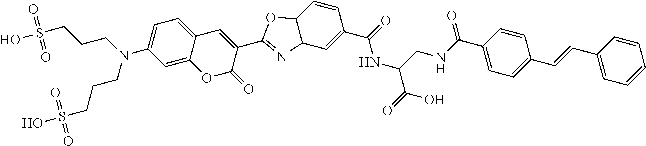 [SMILES: O=C(NC(CNC(=O)c1ccc(/C=C/c2ccccc2)cc1)C(=O)O)C1=CC2N=C(c3cc4ccc(N(CCCS(=O)(=O)O)CCCS(=O)(=O)O)cc4oc3=O)OC2C=C1] |
| Compound 6A | 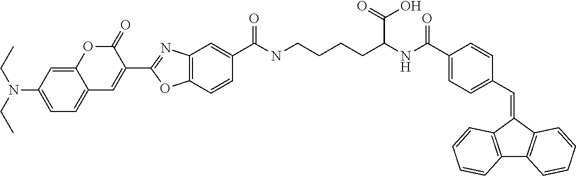 |
| Compound 6B | 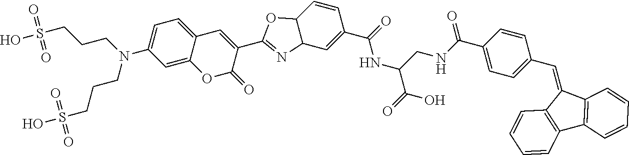 [SMILES: O=C(NC(CNC(=O)c1ccc(C=C2c3ccccc3-c3ccccc32)cc1)C(=O)O)C1=CC2N=C(c3cc4ccc(N(CCCS(=O)(=O)O)CCCS(=O)(=O)O)cc4oc3=O)OC2C=C1] |
| Compound 7A | 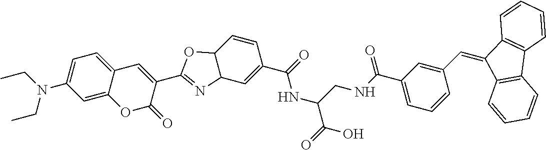 [SMILES: CCN(CC)c1ccc2cc(C3=NC4C=C(C(=O)NC(CNC(=O)c5cccc(C=C6c7ccccc7-c7ccccc76)c5)C(=O)O)C=CC4O3)c(=O)oc2c1] |
| Compound 7B | 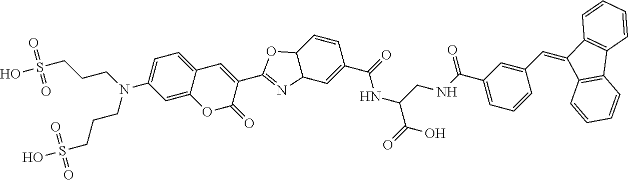 [SMILES: O=C(NC(CNC(=O)c1cccc(C=C2c3ccccc3-c3ccccc32)c1)C(=O)O)C1=CC2N=C(c3cc4ccc(N(CCCS(=O)(=O)O)CCCS(=O)(=O)O)cc4oc3=O)OC2C=C1] |

### Fluorescent Dyes

The photoprotective moieties described herein may be covalently attached to various fluorescent dyes suitable for sequencing analysis, for example, sequencing by synthesis technologies for the purpose of preventing or reducing photobleaching of the dye and/or fluorescent signal decay during sequencing runs. In particular, the fluorescent compounds can be excited by a light source having a wavelength from about 400 nm to about 500 nm, about 420 nm to about 480 nm, or about 450 nm to about 460 nm. Dyes that can be excited by a blue light are also referred to as “blue dyes.” These compounds have been described in U.S. Publication Nos. 2018/0094140 A1, 2018/0201981 A1, 2020/0277529 A1, 2020/0277670 A1, 2021/0188832 A1, 2022/0033900 A1, 2022/0195196 A1, 2022/0195517 A1, 2022/0380389 A1, as well as U.S. Ser. Nos. 18/342064 and 18/190531, each of which is incorporated by reference in its entirety.

### Labeled Nucleotides

According to an aspect of the disclosure, the photo-protected fluorescent compounds containing one or more photoprotective moieties of formula (Ia), (Ia-1), (Ia-2), (Ib), (IIa), (IIa-1), (IIa-2), (IIb), (IIb-1), (III), (III-1), (IV), (IV-1) or (IV-2) as described herein are suitable for attachment to substrate moieties, particularly comprising linker groups to enable attachment to substrate moieties. Substrate moieties can be virtually any molecule or substance to which the dyes of the disclosure can be conjugated, and, by way of non-limiting example, may include nucleosides, nucleotides, polynucleotides, carbohydrates, ligands, particles, solid surfaces, organic and inorganic polymers, chromosomes, nuclei, living cells, and combinations or assemblages thereof. The fluorescent compounds can be conjugated by an optional linker by a variety of means including hydrophobic attraction, ionic attraction, and covalent attachment. In some aspect, the fluorescent compounds are conjugated to the substrate by covalent attachment. More particularly, the covalent attachment is by means of a linker group. In some instances, such labeled nucleotides are also referred to as “modified nucleotides.”

Some aspects of the present disclosure relate to a nucleotide labeled with a photo-protected fluorescent compound containing one or more photoprotective moieties as described herein, or salts or mesomeric form thereof. The labeled nucleotide may be attached to the fluorescent compound via the reaction of a carboxyl (—CO₂H) with an amino (—NH₂) to form an amide bond. In some further embodiments, the carboxyl group may be in the form of an activated form of carboxyl group, for example, an amide or ester, which may be used for attachment to an amino or hydroxyl group of the nucleotide. The term “activated ester” as used herein, refers to a carboxyl group derivative which is capable of reacting in mild conditions, for example, with a compound containing an amino group. Non-limiting examples of activated esters include but not limited to p-nitrophenyl, pentafluorophenyl and succinimido esters.

In some embodiments, the fluorescent compound may be covalently attached to a nucleotide via the nucleotide base. In some such embodiments, the labeled nucleotide may have the fluorescent compound attached to the C₅ position of a pyrimidine base or the C₇ position of a 7-deaza purine base, optionally through a linker moiety. For example, the nucleobase may be 7-deaza adenine, and the dye is attached to the 7-deaza adenine at the C₇ position, optionally through a linker. The nucleobase may be 7-deaza guanine, and the dye is attached to the 7-deaza guanine at the C₇ position, optionally through a linker. The nucleobase may be cytosine and the dye is attached to the cytosine at the C₅ position, optionally through a linker. As another example, the nucleobase may be thymine or uracil and the dye is attached to the thymine or uracil at the C₅ position, optionally through a linker.

### 3′ Blocking Groups

The labeled nucleotide may also have a blocking group covalently attached to the ribose or deoxyribose sugar of the nucleotide. The blocking group may be attached at any position on the ribose or deoxyribose sugar. In particular embodiments, the blocking group is at the 3′OH position of the ribose or deoxyribose sugar of the nucleotide. Various 3′ OH blocking group are disclosed in WO2004/018497 and WO2014/139596, which are hereby incorporated by references. For example, the blocking group may be azidomethyl (—CH₂N₃) or substituted azidomethyl (e.g., —CH(CHF₂)N₃ or —CH(CH₂F)N₃), or allyl connecting to the 3′ oxygen atom of the ribose or deoxyribose moiety. In some embodiments, the 3′ blocking group is azidomethyl, forming 3′-OCH₂N₃ with the 3′ carbon of the ribose or deoxyribose.

Additional 3′ blocking groups are disclosed in U.S. Publication No. 2020/0216891 A1, which is incorporated by reference in its entirety. Non-limiting examples of the 3′ blocking group include:

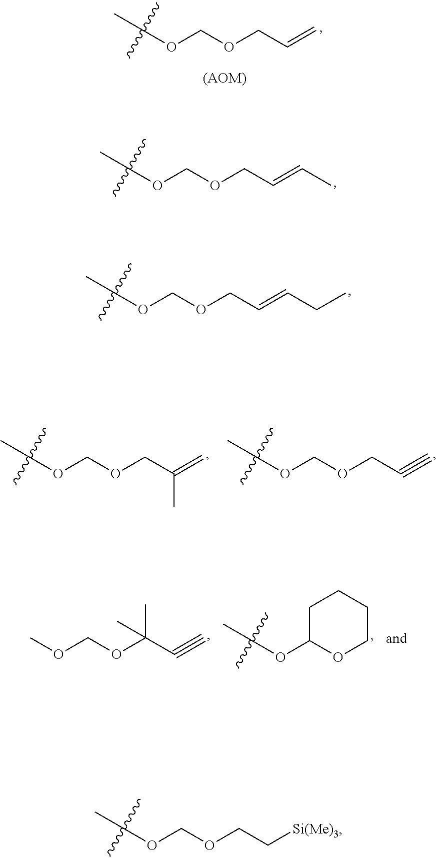

each covalently attached to the 3′ carbon of the ribose or deoxyribose.

### Deprotection of the 3′ Blocking Groups

In some embodiments, the 3′ blocking group may be removed or deprotected by a chemical reagent to generate a free hydroxy group, for example, in the presence of a water soluble phosphine reagent. Non-limiting examples include tris(hydroxymethyl)phosphine (THMP), tris(hydroxyethyl)phosphine (THEP) or tris(hydroxylpropyl)phosphine (THP or THPP). 3′-acetal blocking groups described herein may be removed or cleaved under various chemical conditions. For 3′ acetal blocking groups such as

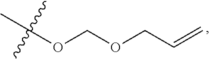 [SMILES: *OCOCC=C]

non-limiting cleaving condition includes a Pd(II) complex, such as Pd(OAc)₂ or allylPd(II) chloride dimer, in the presence of a phosphine ligand, for example tris(hydroxymethyl)phosphine (THMP), or tris(hydroxylpropyl)phosphine (THP or THPP). For those blocking groups containing an alkynyl group (e.g., an ethynyl), they may also be removed by a Pd(II) complex (e.g., Pd(OAc)₂ or allyl Pd(II) chloride dimer) in the presence of a phosphine ligand (e.g., THP or THMP).

### Palladium Cleavage Reagents

In some other embodiments, the 3′ blocking group such as allyl or AOM as described herein may be cleaved by a palladium catalyst. In some such embodiments, the Pd catalyst is water soluble. In some such embodiments, is a Pd(0) complex (e.g., Tris(3,3′,3″-phosphinidynetris(benzenesulfonato)palladium(0) nonasodium salt nonahydrate). In some instances, the Pd(0) complex may be generated in situ from reduction of a Pd(II) complex by reagents such as alkenes, alcohols, amines, phosphines, or metal hydrides. Suitable palladium sources include Na₂PdCl₄, Li₂PdCl₄, Pd(CH₃CN)₂Cl₂, (PdCl(C₃H₅))₂, [Pd(C₃H₅)(THP)]Cl, [Pd(C₃H₅)(THP)₂]Cl, Pd(OAc)₂, Pd(Ph₃)₄, Pd(dba)₂, Pd(Acac)₂, PdCl2(COD), Pd(TFA)₂, Na₂PdBr₄, K₂PdBr₄, PdCl₂, PdBr₂, and Pd(NO₃)₂. In one such embodiment, the Pd(0) complex is generated in situ from Na₂PdCl₄ or K₂PdCl₄. In another embodiment, the palladium source is allyl palladium(II) chloride dimer [(PdCl(C₃H₅))₂]. In some embodiments, the Pd(0) complex is generated in an aqueous solution by mixing a Pd(II) complex with a phosphine. Suitable phosphines include water soluble phosphines, such as tris(hydroxypropyl)phosphine (THP), tris(hydroxymethyl)phosphine (THMP), 1,3,5-triaza-7-phosphaadamantane (PTA), bis(p-sulfonatophenyl)phenylphosphine dihydrate potassium salt, tris(carboxyethyl)phosphine (TCEP), and triphenylphosphine-3,3′,3″-trisulfonic acid trisodium salt.

In some embodiments, the palladium catalyst is prepared by mixing [(Allyl)PdCl]₂ with THP in situ. The molar ratio of [(Allyl)PdCl]₂ and the THP may be about 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5 or 1:10. In one embodiment, the molar ratio of [(Allyl)PdCl]₂ to THP is 1:10. In some other embodiment, the palladium catalyst is prepared by mixing a water soluble Pd reagent such as Na₂PdCl₄ or K₂PdCl₄ with THP in situ. The molar ratio of Na₂PdC₁₄ or K₂PdCl₄ and THP may be about 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5 or 1:10. In one embodiment, the molar ratio of Na₂PdCl₄ or K₂PdCl₄ to THP is about 1:3. In another embodiment, the molar ratio of Na₂PdCl₄ or K₂PdCl₄ to THP is about 1:3.5. In yet another embodiment, the molar ratio of Na₂PdCl₄ or K₂PdCl₄ to THP is about 1:2.5. In some further embodiments, one or more reducing agents may be added, such as ascorbic acid or a salt thereof (e.g., sodium ascorbate). In some embodiments, the cleavage mixture may contain additional buffer reagents, such as a primary amine, a secondary amine, a tertiary amine, a carbonate salt, a phosphate salt, or a borate salt, or combinations thereof. In some further embodiments, the buffer reagent comprises ethanolamine (EA), tris(hydroxymethyl)aminomethane (Tris), glycine, sodium carbonate, sodium phosphate, sodium borate, 2-dimethylethanolamine (DMEA), 2-diethylethanolamine (DEEA), N,N,N′,N′-tetramethylethylenediamine(TEMED), or N,N,N′,N′-tetraethylethylenediamine (TEEDA), or 2-piperidine ethanol (also known as (2-hydroxyethyl)piperidine, having the structure

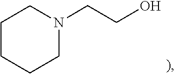

or combinations thereof. In one embodiment, the buffer reagent comprises or is DEEA. In another embodiment, the buffer reagent comprises or is (2-hydroxyethyl)piperidine. In another embodiment, the buffer reagent contains one or more inorganic salts such as a carbonate salt, a phosphate salt, or a borate salt, or combinations thereof. In one embodiment, the inorganic salt is a sodium salt.

### Palladium (Pd) Scavengers

Pd has the capacity to stick on DNA, mostly in its inactive Pd(II) form, which may interfere with the binding between DNA and polymerase, causing increased phasing. A post-cleavage wash composition that includes a Pd scavenger compound may be used following the deblocking step. For example, PCT Publication No. WO 2020/126593 discloses Pd scavengers such as 3,3′-dithiodipropionic acid (DDPA) and lipoic acid (LA) may be included in the scan composition and/or the post-cleavage wash composition. The use of these scavengers in the post-cleave washing solution has the purpose of scavenging Pd(0), converting Pd(0) to the inactive Pd(II) form, thereby improving the prephasing value and sequencing metrics, reducing signal degrade, and extend sequencing read length.

### Cleavable Linkers

The photo-protected fluorescent compounds as disclosed herein may include a reactive group (e.g., a carboxyl group, either on the fluorescent compound itself, or on the one or more photoprotective moieties covalently attached thereof) for covalent attachment of the photo-protected fluorescent compound to a substrate, such as a nucleotide with a reactive linker group (e.g., a linker group with an amino group). In a particular embodiment the reactive linker may be a cleavable linker. Use of the term “cleavable linker” is not meant to imply that the whole linker is required to be removed. The cleavage site can be located at a position on the linker that ensures that part of the linker remains attached to the dye and/or substrate moiety after cleavage. Cleavable linkers may be, by way of non-limiting example, electrophilically cleavable linkers, nucleophilically cleavable linkers, photocleavable linkers, cleavable under reductive conditions (for example disulfide or azide containing linkers), oxidative conditions, cleavable via use of safety-catch linkers and cleavable by elimination mechanisms. The use of a cleavable linker to attach the dye compound to a substrate moiety ensures that the label can, if required, be removed after detection, avoiding any interfering signal in downstream steps.

Useful linker groups may be found in PCT Publication No. WO2004/018493 (herein incorporated by reference), examples of which include linkers that may be cleaved using water-soluble phosphines or water-soluble transition metal catalysts formed from a transition metal and at least partially water-soluble ligands. In aqueous solution the latter form at least partially water-soluble transition metal complexes. Such cleavable linkers can be used to connect bases of nucleotides to labels such as the dyes set forth herein.

Particular linkers include those disclosed in PCT Publication No. WO2004/018493 (herein incorporated by reference) such as those that include moieties of the formulae:

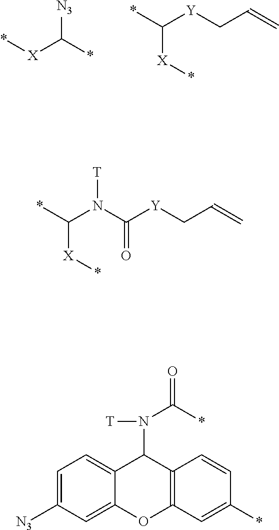

(wherein X is selected from the group comprising O, S, NH and NQ wherein Q is a C₁-10 substituted or unsubstituted alkyl group, Y is selected from the group comprising O, S, NH and N(allyl), T is hydrogen or a C₁-C₁₀ substituted or unsubstituted alkyl group and * indicates where the moiety is connected to the remainder of the nucleotide or nucleoside). In some aspect, the linkers connect the bases of nucleotides to labels such as, for example, the dye compounds described herein.

Additional examples of linkers include those disclosed in U.S. Publication No. 2016/0040225 (herein incorporated by reference), such as those include moieties of the formulae:

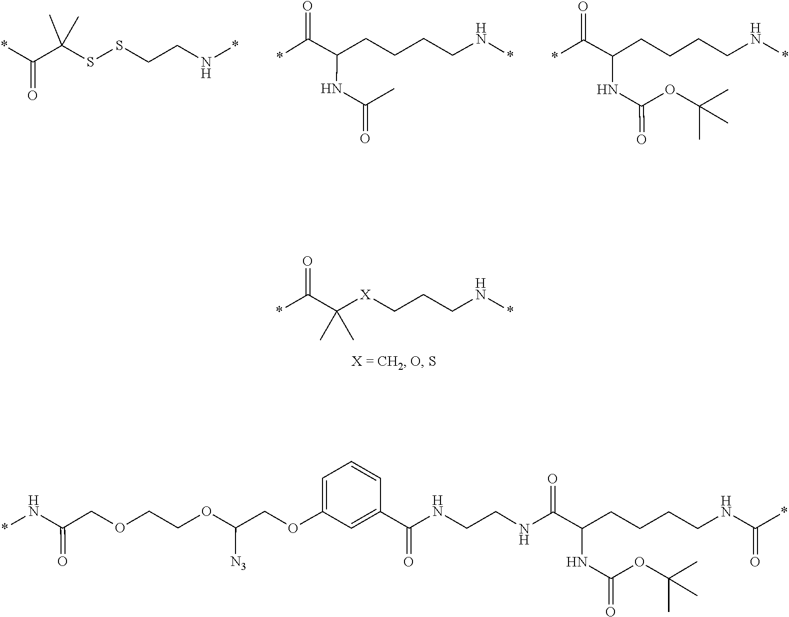

(wherein * indicates where the moiety is connected to the remainder of the nucleotide or nucleoside). The linker moieties illustrated herein may comprise the whole or partial linker structure between the nucleotides/nucleosides and the labels. The linker moieties illustrated herein may comprise the whole or partial linker structure between the nucleotides/nucleosides and the labels.

Additional examples of linkers include moieties of the formula:

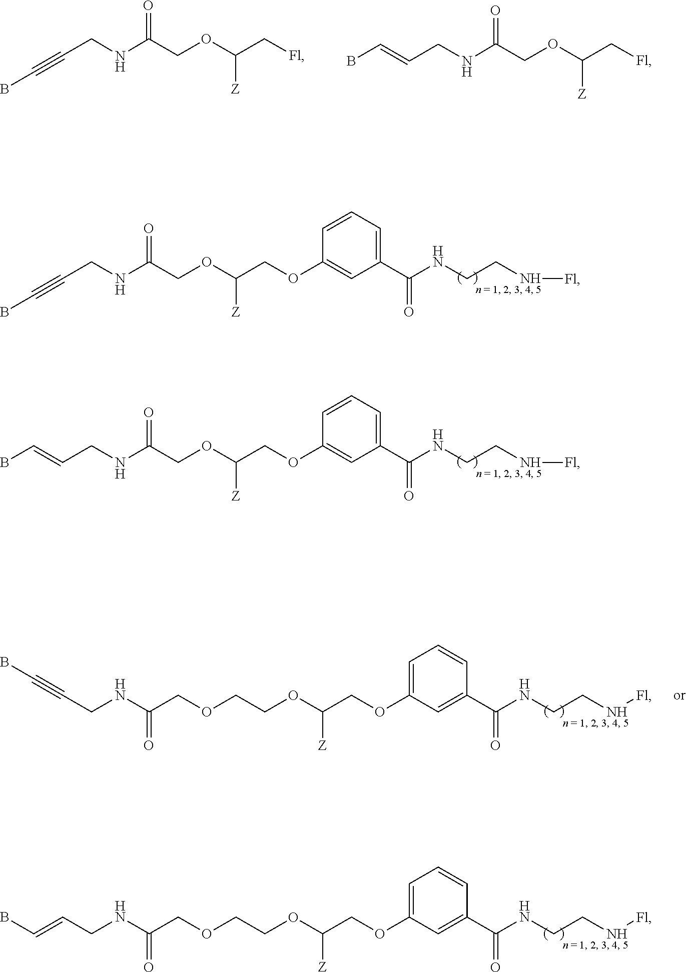

wherein B is a nucleobase; Z is —N₃ (azido), —O—C₁-C₆ alkyl, —O—C₂-C₆ alkenyl, or —O—C₂-C₆ alkynyl; and Fl comprises a photoprotected fluorescent compound moiety, which may contain additional linker structure. One of ordinary skill in the art understands that the dye compound described herein is covalently bounded to the linker by reacting a functional group of the dye compound (e.g., carboxyl) with a functional group of the linker (e.g., amino). In one embodiment, the cleavable linker comprises

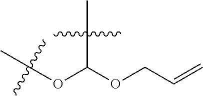 [SMILES: *OC(*)OCC=C]

(“AOL” linker moiety) where Z is —O-allyl. In addition, the nucleotide may contain multiple cleavable linkers repeating units.

In particular embodiments, the length of the linker between a fluorescent compound (fluorophore) and a guanine base can be altered, for example, by introducing a polyethylene glycol spacer group, thereby increasing the fluorescence intensity compared to the same fluorophore attached to the guanine base through other linkages known in the art. Exemplary linkers and their properties are set forth in PCT Publication No. WO2007020457 (herein incorporated by reference). The design of linkers, and especially their increased length, can allow improvements in the brightness of fluorophores attached to the guanine bases of guanosine nucleotides when incorporated into polynucleotides such as DNA. Thus, when the dye is for use in any method of analysis which requires detection of a fluorescent dye label attached to a guanine-containing nucleotide, it is advantageous if the linker comprises a spacer group of formula —((CH₂)₂O)ₙ—, wherein n is an integer between 2 and 50, as described in WO 2007/020457.

Nucleosides and nucleotides may be labeled at sites on the sugar or nucleobase. As known in the art, a “nucleotide” consists of a nitrogenous base, a sugar, and one or more phosphate groups. In RNA, the sugar is ribose and in DNA is a deoxyribose, i.e., a sugar lacking a hydroxy group that is present in ribose. The nitrogenous base is a derivative of purine or pyrimidine. The purines are adenine (A) and guanine (G), and the pyrimidines are cytosine (C) and thymine (T) or in the context of RNA, uracil (U). The C-1 atom of deoxyribose is bonded to N-1 of a pyrimidine or N-9 of a purine. A nucleotide is also a phosphate ester of a nucleoside, with esterification occurring on the hydroxy group attached to the C-3 or C-5 of the sugar. Nucleotides are usually mono, di- or triphosphates.

A “nucleoside” is structurally similar to a nucleotide but is missing the phosphate moieties. An example of a nucleoside analog would be one in which the label is linked to the base and there is no phosphate group attached to the sugar molecule.

Although the base is usually referred to as a purine or pyrimidine, the skilled person will appreciate that derivatives and analogues are available which do not alter the capability of the nucleotide or nucleoside to undergo Watson-Crick base pairing. “Derivative” or “analogue” means a compound or molecule whose core structure is the same as, or closely resembles that of a parent compound but which has a chemical or physical modification, such as, for example, a different or additional side group, which allows the derivative nucleotide or nucleoside to be linked to another molecule. For example, the base may be a deazapurine. In particular embodiments, the derivatives should be capable of undergoing Watson-Crick pairing. “Derivative” and “analogue” also include, for example, a synthetic nucleotide or nucleoside derivative having modified base moieties and/or modified sugar moieties. Such derivatives and analogues are discussed in, for example, Scheit, Nucleotide analogs (John Wiley & Son, 1980) and Uhlman et al., Chemical Reviews 90:543-584, 1990. Nucleotide analogues can also comprise modified phosphodiester linkages including pho sphorothio ate, pho sphorodithio ate, alkyl-phosphonate, phosphoranilidate, phosphoramidate linkages and the like.

A dye may be attached to any position on the nucleotide base, for example, through a linker. In particular embodiments, Watson-Crick base pairing can still be carried out for the resulting analog. Particular nucleobase labeling sites include the C5 position of a pyrimidine base or the C7 position of a 7-deaza purine base. As described above a linker group may be used to covalently attach a dye to the nucleoside or nucleotide.

In particular embodiments the labeled nucleotide or oligonucleotide may be enzymatically incorporable and enzymatically extendable. Accordingly, a linker moiety may be of sufficient length to connect the nucleotide to the compound such that the compound does not significantly interfere with the overall binding and recognition of the nucleotide by a nucleic acid replication enzyme. Thus, the linker can also comprise a spacer unit. The spacer distances, for example, the nucleotide base from a cleavage site or label.

Nucleosides or nucleotides labeled with the dyes described herein may have the formula:

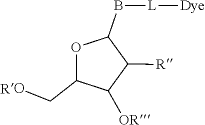

where Dye is a photo-protected fluorescent compound (label) moiety described herein (after covalent bonding between a functional group of the photo-protected fluorescent compound and a functional group of the linker “L”); B is a nucleobase, such as, for example uracil, thymine, cytosine, adenine, 7-deaza adenine, guanine, 7-deaza guanine, and the like; L is an optional linker which may or may not be present; R′ can be H, or —OR′ is monophosphate, diphosphate, triphosphate, thiophosphate, a phosphate ester analog, —O— attached to a reactive phosphorous containing group, or —O— protected by a blocking group; R″ is H or OH; and R″′ is H, a 3′ blocking group described herein, or —OR″′ forms a phosphoramidite. Where —OR″′ is phosphoramidite, R′ is an acid-cleavable hydroxyl protecting group which allows subsequent monomer coupling under automated synthesis conditions. In some further embodiments, B comprises

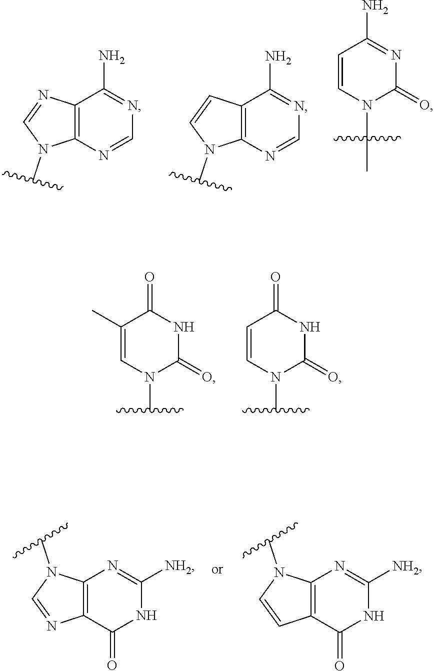

or optionally substituted derivatives and analogs thereof. In some further embodiments, the labeled nucleobase comprises the structure

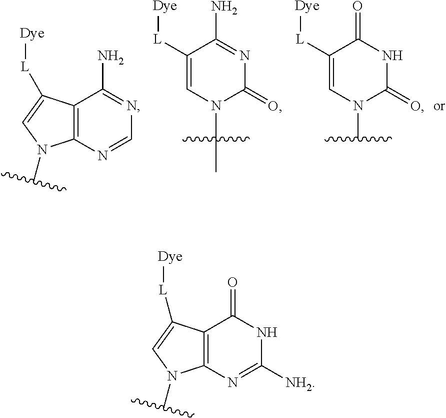

In yet another alternative embodiment, there is no blocking group on the 3′ carbon of the pentose sugar and the fluorescent compound attached to the base, for example, can be of a size or structure sufficient to act as a block to the incorporation of a further nucleotide. Thus, the block can be due to steric hindrance or can be due to a combination of size, charge and structure, whether or not the dye is attached to the 3′ position of the sugar.

In still yet another alternative embodiment, the blocking group is present on the 2′ or 4′ carbon of the pentose sugar and can be of a size or structure sufficient to act as a block to the incorporation of a further nucleotide.

The use of a blocking group allows polymerization to be controlled, such as by stopping extension when a labeled nucleotide is incorporated. If the blocking effect is reversible, for example, by way of non-limiting example by changing chemical conditions or by removal of a chemical block, extension can be stopped at certain points and then allowed to continue.

In a particular embodiment, the linker (between the photo-protected fluorescent compound and nucleotide) and blocking group are both present and are separate moieties. In particular embodiments, the linker and blocking group are both cleavable under the same or substantially similar conditions. Thus, deprotection and deblocking processes may be more efficient because only a single treatment will be required to remove both the dye compound and the blocking group. However, in some embodiments a linker and blocking group need not be cleavable under similar conditions, instead being individually cleavable under distinct conditions.

The disclosure also encompasses polynucleotides incorporating dye compounds. Such polynucleotides may be DNA or RNA comprised respectively of deoxyribonucleotides or ribonucleotides joined in phosphodiester linkage. Polynucleotides may comprise naturally occurring nucleotides, non-naturally occurring (or modified) nucleotides other than the labeled nucleotides described herein or any combination thereof, in combination with at least one modified nucleotide (e.g., labeled with a dye compound) as set forth herein. Polynucleotides according to the disclosure may also include non-natural backbone linkages and/or non-nucleotide chemical modifications. Chimeric structures comprised of mixtures of ribonucleotides and deoxyribonucleotides comprising at least one labeled nucleotide are also contemplated.

Non-limiting exemplary labeled nucleotides as described herein include:

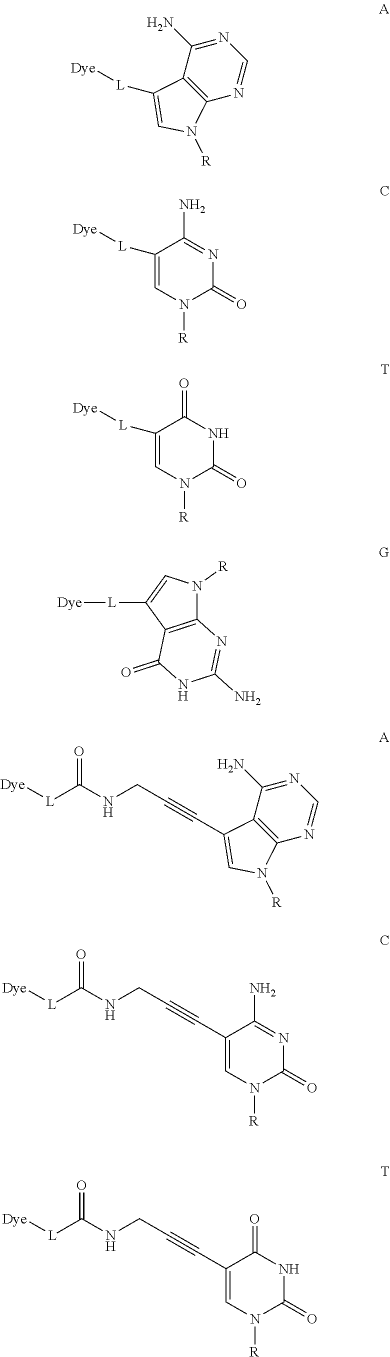

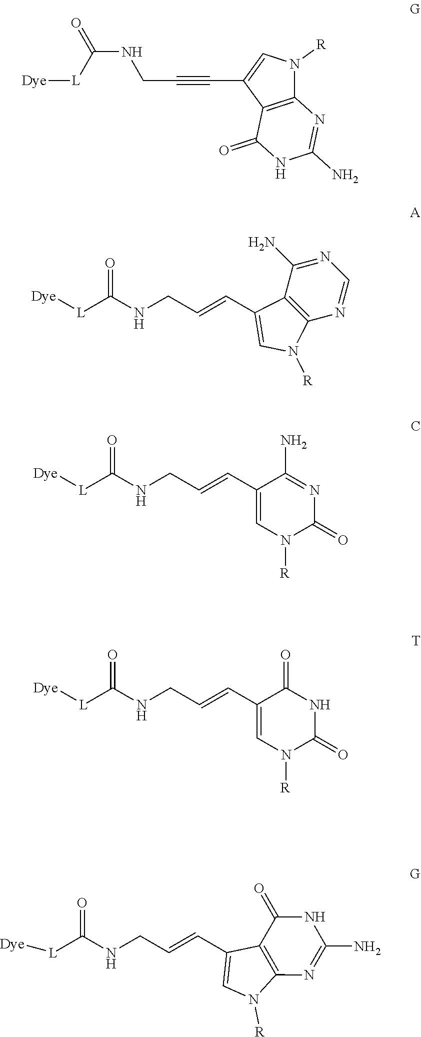

wherein L represents a linker and R represents a ribose or deoxyribose moiety as described above, or a ribose or deoxyribose moiety with the 5′ position substituted with mono-, di- or tri-phosphates.

In some embodiments, non-limiting exemplary fluorescent dye conjugates are shown below:

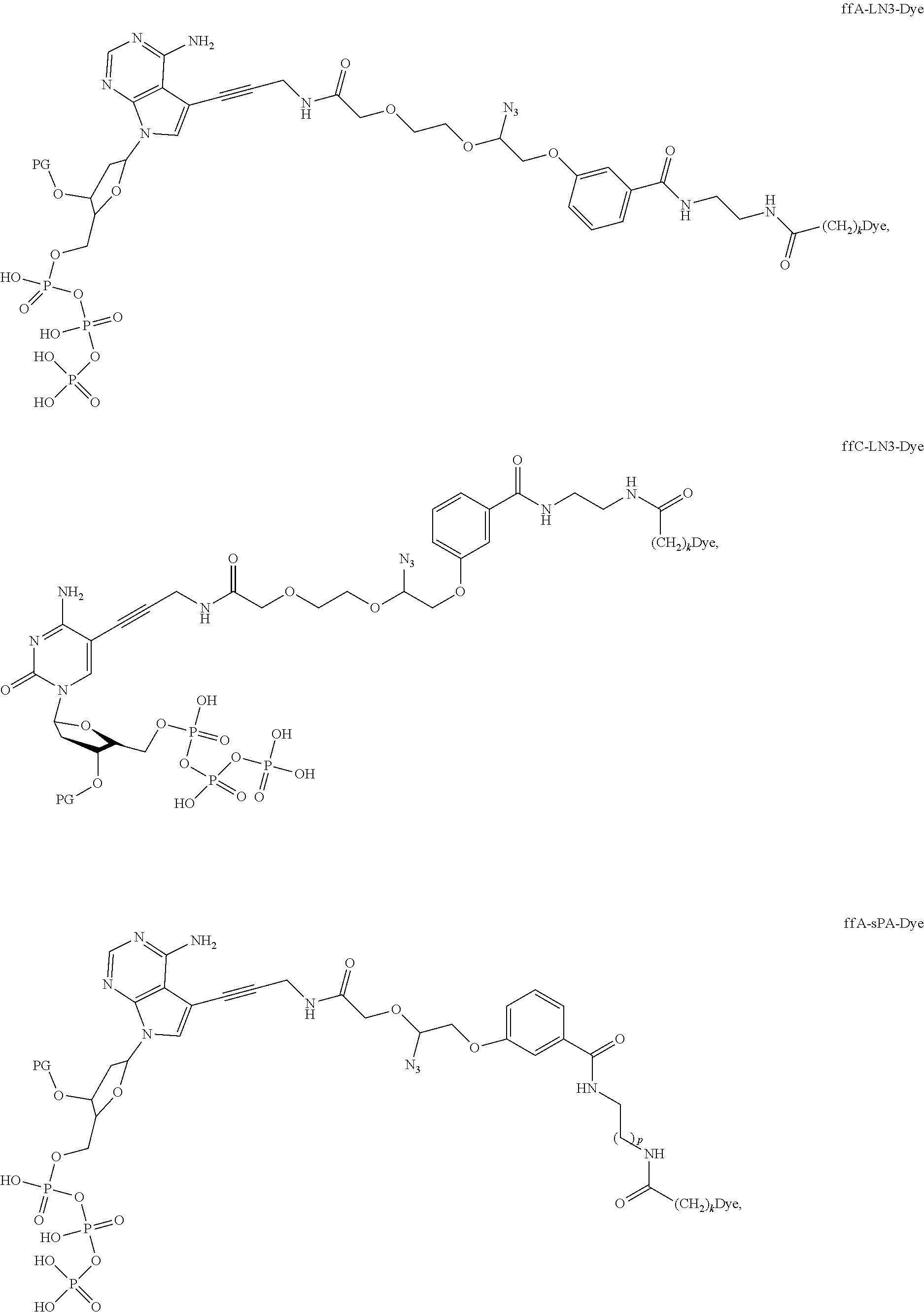

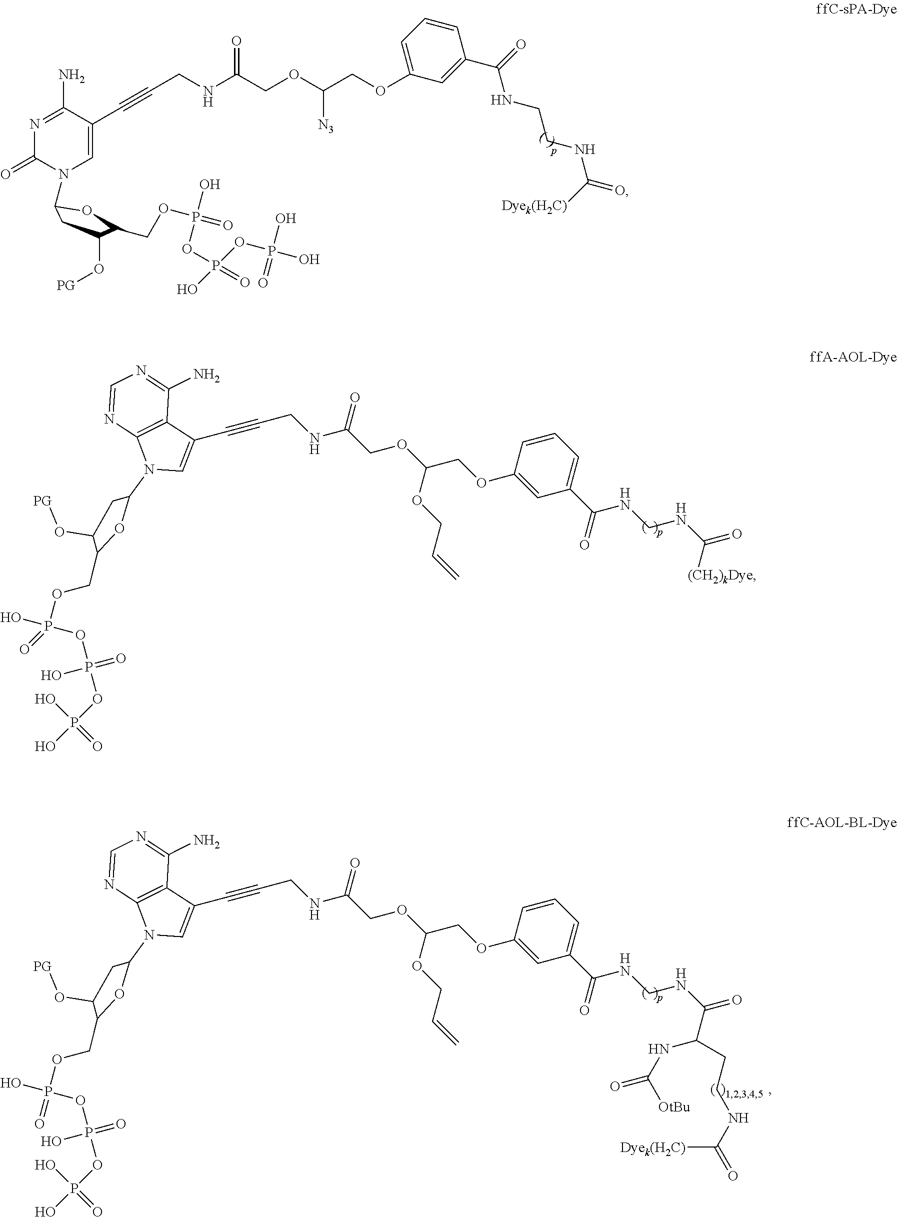

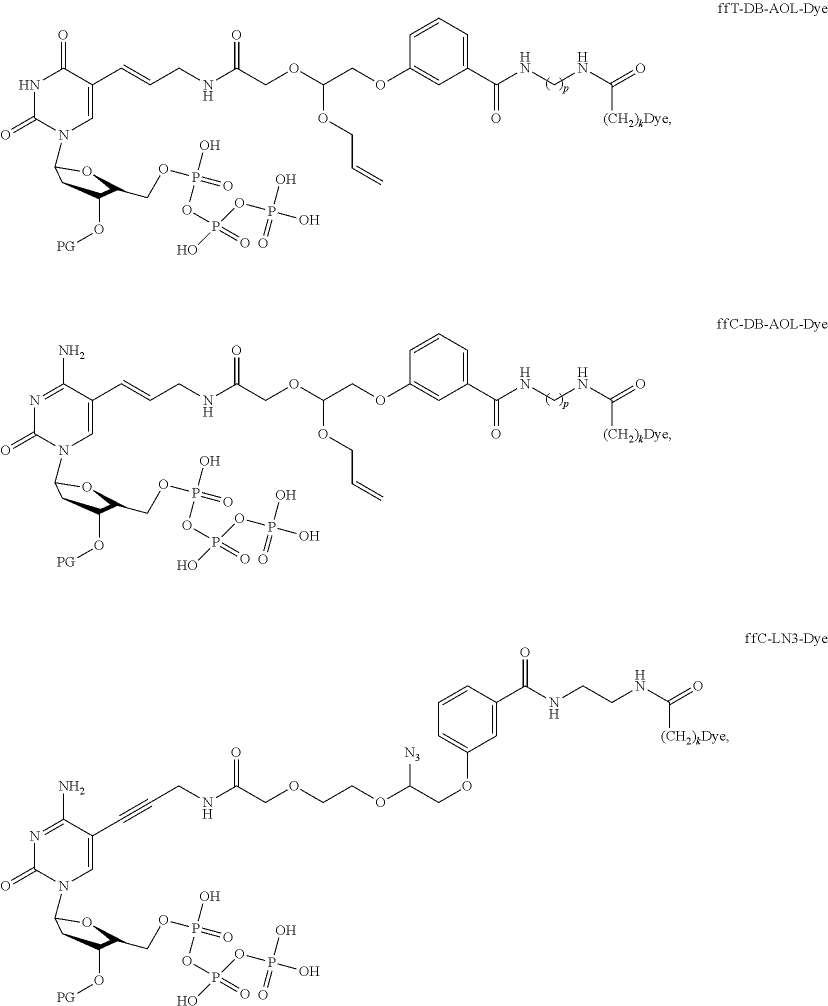

wherein PG stands for the 3′ OH blocking groups described herein; p is an integer of 1, 2, 3, 4, 5, 6, 7, 8, 9, or 10; and k is 0, 1, 2, 3, 4, or 5. In one embodiment, —O—PG is AOM. In another embodiment, —O—PG is —O—azidomethyl. In one embodiment, k is 5. In some further embodiments, p is 1, 2 or 3; and k is 5.

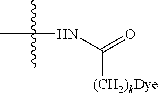

refers to the connection point of the Dye with the cleavable linker as a result of a reaction between an amino group of the linker moiety and the carboxyl group of the Dye. In any embodiments of the labeled nucleotide described herein, the nucleotide is a nucleoside triphosphate.

### Synthesis of Dyes with Photoprotective Moieties and Labeled Nucleosides

Some aspect of the present disclosure relates to a method of preparing a fluorescent compound covalently attached to one or more photoprotective moieties of the formula (Ia), (Ia-1), (Ia-2), (Ib), (IIa), (IIa-1), (IIa-2), (IIb), (IIb-1), (III), (III-1), (IV), (IV-1) or (IV-2) as described herein. The preparation of a labeled nucleoside is also described. A specific example of a general synthetic scheme is illustrated below:

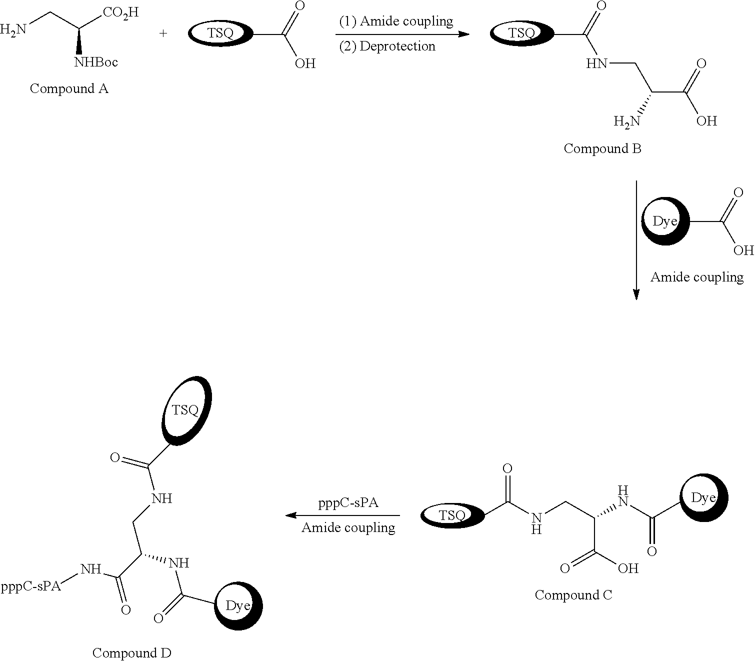

where TSQ refers to the photoprotective starting material having a carboxyl group, which undergoes amide coupling with Compound A, followed by deprotection of the Boc protecting group to form Compound B. Compound B then undergoes amide coupling through reacting an amino group of Compound B with a carboxyl group of the dye to form a photoprotective dye compound C. Furthermore, Compound C can undergo amide coupling through reacting a carboxyl group of Compound C with an amino group of a C nucleoside triphosphate (pppC) containing a sPA linker to form labeled nucleotide Compound D. In addition to the photoprotective moieties described herein, additional photoprotective moieties may include but not limited to gallic acid moieties, trolox moieties, and caffeic acid, where a carboxy group of the compound may be used for forming a covalent bond with a fluorescent dye, optionally through a linker (such as the linker of formula (V) as described herein). The sPA linker is also known as sPA-LN3 linker with structure described herein.

Additional aspects of the present disclosure relate to an oligonucleotide or polynucleotide comprising or incorporating a labeled nucleotide described herein. In some embodiments, the oligonucleotide or polynucleotide is hybridized to at least a portion of a target polynucleotide. In some embodiments, the target polynucleotide is immobilized on a solid support. In some further embodiments, the solid support comprises an array or a plurality of different immobilized target polynucleotides. In further embodiments, the solid support comprises a patterned flow cell. In further embodiments, the patterned flow cell comprises a plurality of nanowells. In further embodiments, the solid support comprises at least 5,000,000 spatially distinguishable sites/cm² that comprise multiple copies of target polynucleotides.

### Kits

Also provided herein are kits including a first type of nucleotide, which is a labeled nucleotide described herein (labeled with a photo-protected fluorescent compound, also called a first label). In some embodiments, the kit also comprises a second type of labeled nucleotide, which is labeled with a second compound that is different than the first label (i.e., a second label). In some embodiments, the first and second type labeled nucleotides are excitable using a single excitation source, which may be a first light source having a first excitation wavelength. For example, the excitation bands for the first and the second labels may be at least partially overlapping such that excitation in the overlap region of the spectrum causes both labels to emit fluorescence. In some other embodiments, the second type of labeled nucleotides is excitable using a second excitation source, which may be a second light source having a second excitation wavelength that is different from the first excitation wavelength. In some further embodiments, the kit may include a third type nucleotide, wherein the third nucleotide is labeled with a third compound that is different from the first and the second labels (i.e., a third label). Alternatively, the third type of nucleotide is labeled with both the first label and the second label. In some such embodiments, the third type labeled nucleotide is excitable using the first light source having the first excitation wavelength. That is, each of the first type, second type and the third type of nucleotide is excitable using the same light source with a single wavelength. In some other embodiments, the third type labeled nucleotide is excitable using both the first light source having the first excitation wavelength, or the second light source having the second excitation wavelength. In still other embodiments, the third type of nucleotide is excitable using a third light source with a third excitation wavelength. In some further embodiments, the kit may further comprise a fourth type of nucleotide. In some such embodiments, the fourth nucleotide is unlabeled (dark). In other embodiments, the fourth nucleotide is labeled with a different compound than the first, second and the third nucleotide, and each label has a distinct absorbance maximum that is distinguishable from the other labels. In still other embodiments, the fourth nucleotide is unlabeled. In some embodiments, the first light source has an excitation wavelength from about 400 nm to about 480 nm, from about 420 nm to about 470 nm, or from 450 nm to about 460 nm (e.g., 450 nm). The second excitation light source has a wavelength from about 500 nm to about 550 nm, from about 510 to about 540 nm, or from about 520 to about 530 nm (e.g., 520 nm). The second light source has an excitation wavelength from about 400 nm to about 480 nm, from about 420nm to about 470 nm, or from 450 nm to about 460 nm (e.g., about 452 nm). In some embodiments, the emissions of the first type of labeled nucleotide, the second type of labeled nucleotide and the third type of labeled nucleotide are detectable in two detection channels with different wavelengths (e.g., at blue region with a wavelength ranging from about 472 to about 520 nm, and at a green region with a wavelength ranging from about 540 nm to about 640nm). In other embodiments, each of the first type, the second type and the third type of nucleotide has an emission spectrum that can be collected in a single emission collection filter or channel.

In some embodiments, the kit may contain four types of labeled nucleotides (A, C, G and T or U), where the first of the four nucleotides is labeled with a photo-protected fluorescent compound as disclosed herein. In such a kit, each of the four nucleotides can be labeled with a compound that is the same or different from the label on the other three nucleotides. Alternatively, a first of the four nucleotides is a labeled nucleotide describe herein, a second of the four nucleotides carries a second label, a third nucleotide carries a third label, and a fourth nucleotide is unlabeled (dark). As another example, a first of the four nucleotides is a labeled nucleotide described herein, a second of the four nucleotides carries a second label, a third nucleotide carries a mixture of two labels, and a fourth nucleotide is unlabeled (dark). Thus, one or more of the label compounds can have a distinct absorbance maximum and/or emission maximum such that the compound(s) is(are) distinguishable from other compounds. For example, each compound can have a distinct absorbance maximum and/or emission maximum such that each of the compounds is spectrally distinguishable from the other three compounds (or two compounds if the fourth nucleotide is unlabeled). It will be understood that parts of the absorbance spectrum and/or emission spectrum other than the maxima can differ and these differences can be exploited to distinguish the compounds. The kit may be such that two or more of the compounds have a distinct absorbance maximum.

The compounds, nucleotides, or kits that are set forth herein may be used to detect, measure, or identify a biological system (including, for example, processes or components thereof). Exemplary techniques that can employ the compounds, nucleotides or kits include sequencing, expression analysis, hybridization analysis, genetic analysis, RNA analysis, cellular assay (e.g., cell binding or cell function analysis), or protein assay (e.g., protein binding assay or protein activity assay). The use may be on an automated instrument for carrying out a particular technique, such as an automated sequencing instrument. The sequencing instrument may contain two light sources operating at different wavelengths.

In a particular embodiment, the labeled nucleotide(s) described herein may be supplied in combination with unlabeled or native nucleotides, or any combination thereof. Combinations of nucleotides may be provided as separate individual components (e.g., one nucleotide type per vessel or tube) or as nucleotide mixtures (e.g., two or more nucleotides mixed in the same vessel or tube).

Where kits comprise a plurality, particularly two, or three, or more particularly four, nucleotides, the different nucleotides may be labeled with different dye compounds, or one may be dark, with no dye compounds. Where the different nucleotides are labeled with different dye compounds, it is a feature of the kits that the dye compounds are spectrally distinguishable fluorescent dyes. As used herein, the term “spectrally distinguishable fluorescent dyes” refers to fluorescent compounds that emit fluorescent energy at wavelengths that can be distinguished by fluorescent detection equipment (for example, a commercial capillary-based DNA sequencing platform) when two or more such fluorescent compounds are present in one sample. When two nucleotides labeled with fluorescent compounds are supplied in kit form, it is a feature of some embodiments that the spectrally distinguishable fluorescent compounds can be excited at the same wavelength, such as, for example by the same light source. When four nucleotides labeled with fluorescent compounds are supplied in kit form, it is a feature of some embodiments that two of the spectrally distinguishable fluorescent compounds can both be excited at one wavelength and the other two spectrally distinguishable compounds can both be excited at another wavelength. Particular excitation wavelengths for the dyes are between 450-460 nm, 490-500 nm, or 520 nm or above (e.g., 532 nm).

Although kits are exemplified herein in regard to configurations having different nucleotides that are labeled with different dye compounds, it will be understood that kits can include 2, 3, 4 or more different nucleotides that have the same fluorescent compound.

Another aspect of the disclosure relates to a kit for use with a sequencing apparatus, comprising a scan mixture composition, wherein the scan mixture composition comprises one or more additives for reducing fluorescent signal decay caused by the fluorescent measurements, and wherein the one or more additives comprise a tri-substituted 1,4-cyclooctatetraene (COT) analog. In other embodiments, the aqueous scan mixture may contain a quaternary ammonium surfactant such as cetyltrimethylammonium bromide (CTAB). CTAB may be used to replace TWEEN, a common surfactant typically used in the universal scan mixture.

In some embodiments of the kit described herein, the tri-substituted 1,4-COT

analog has the structure of formula (VI):

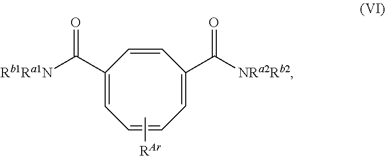

wherein
each of R^{a1}, R^{a2}, R^{b1} and R^{b2} is independently H, unsubstituted C₁-C₆ alkyl, or substituted C₁-C₆ alkyl;

R^{Ar} is carboxyl, C₁-C₆ alkyl, substituted C₁-C₆ alkyl, C₁-C₆ alkoxy, substituted C₁-C₆ alkoxy, C₁-C₆haloalkyl, C₁-C₆haloalkoxy, (C₁-C₆ alkoxy)C₁-C₆ alkyl, —O(C₁-C₆ alkoxy)C₁-C₆ alkyl, optionally substituted amino, amino(C₁-C₆ alkyl), halo, cyano, hydroxy, hydroxy(C₁-C₆ alkyl), nitro, sulfonyl, sulfo, sulfonate, S-sulfonamido, N-sulfonamido, C₆-C₁₀ aryl, 5 to 10 membered heteroaryl, C₃-C₁₀ carbocyclyl, 3 to 10 membered heterocyclyl, —C(═O)OR¹⁰, —C(═O)NR¹¹ R¹²; —(CH₂CH₂O),(C₁-C₆ alkyl), or —O(CH₂CH₂O),(C₁-C₆ alkyl); and
each of R¹⁰, R¹¹ and R¹² is independently H, unsubstituted C₁-C₆ alkyl, or substituted C₁-C₆ alkyl.

In some embodiments of the kit described herein, the tri-substituted 1,4-COT analog has the structure of formula (VI-1):

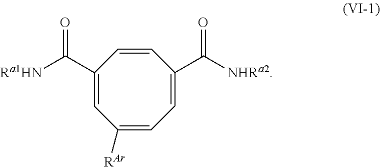

In some such embodiments, R^{Ar} is sulfo, chloro, bromo, nitro, phenyl, C₁-C₆ alkyl (such as methyl, ethyl, isopropyl, propyl, n-butyl, t-butyl), —NH(C₁-C₆ alkyl) (e.g., —NHCH₃ or NHCH₂CH₃), or —N(C₁-C₆ alkyl)₂ (e.g., —N(CH₃)₂, —N(CH₃)(CH₂CH₃) or —N(CH₂CH₃)₂). In some such embodiments, each of R^{a1} and R^{a2} is independently C₁-C₆ alkyl, unsubstituted or substituted with carboxyl, sulfo, or sulfonate. In further embodiments, the scan mixture composition may further comprise a radical scavenger, an oxygen scavenger, a reducing reagent, an antioxidant, or combinations thereof, as described in the method set forth herein in details, including those described in U.S. Ser. No. 18/192288, which is incorporated by reference in its entirety. In some further embodiments, the scan mixture composition may further comprise one or more buffering agents or surfactants, or combinations thereof. The additive(s) in the aqueous scan mixture as described herein may reduce percent signal decay or percent error rate by at least 10%, 20%, 30%, 40%, 50%, 60%, 70%, 80%, 90%, 100%, 150%, 200%, 250%, 300%, 350%, or 400% after at least 50, 100, 150, 200, 250, 300, 350, 400, 450 or 500 cycles of SBS.

In addition to the labeled nucleotides, the kit may comprise at least one additional component. The further component(s) may be one or more of the components identified in a method set forth herein or in the Examples section below. Some non-limiting examples of components that can be combined into a kit of the present disclosure are set forth below. For example, the kit may further comprise an incorporation mixture composition for incorporating 3′ blocked, labeled nucleotides into copy polynucleotide strands complementary to at least a portion of template polynucleotide strands, wherein the incorporation mixture composition comprises: one or more different types of labeled nucleotides, wherein each of the labeled nucleotides comprises a 3′ blocking group; and a DNA polymerase, and/or one or more buffer compositions. Non-limiting examples of DNA polymerase may be used in the present disclosure include those disclosed in WO 2005/024010, US Publication Nos. 2020/0131484 A1 and 2020/0181587 A1, each of which is incorporated by reference herein in its entirety. In further embodiments, the kit may further a cleavage mixture composition, wherein the cleavage mixture composition comprises a reagent for removing the label and the 3′ blocking group of the incorporated nucleotides and/or the cleavable linker. For example, a water-soluble phosphines or water-soluble transition metal catalysts formed from a transition metal and at least partially water-soluble ligands, such as a palladium complex. Various components of the kit may be provided in a concentrated form to be diluted prior to use. In such embodiments a suitable dilution buffer may also be included. Again, one or more of the components identified in a method set forth herein can be included in a kit of the present disclosure. In any embodiments of the nucleotide or labeled nucleotide described herein, the nucleotide contains a 3′ blocking group.

### Methods of Reducing Light-induced Signal Decay

One aspect of the present disclosure relates to a method for reducing light-induced sequencing signal decay during sequencing by synthesis, comprising:
imaging and performing one or more fluorescent measurements of the extended copy polynucleotides in an aqueous scan mixture to determine the identity of the incorporated nucleotides;
wherein the aqueous scan mixture comprises one or more additives for reducing fluorescent signal decay caused by the fluorescent measurements, and wherein the one or more additives comprise a tri-substituted 1,4-cyclooctatetraene (COT) analog.

In particular, the method for reducing light-induced sequencing signal decay during sequencing by synthesis may comprise:
(i) contacting a solid support with an incorporation mixture comprising DNA polymerase and one more of four different types of nucleotides, wherein the solid support comprises a plurality of different target polynucleotides immobilized thereon, and sequencing primers that are complementary and hybridized to at least a portion of the target polynucleotides;
(ii) incorporating one type of nucleotides into the sequencing primers to produce extended copy polynucleotides, wherein one or more four types of nucleotides comprises a detectable label, and each of the four types of nucleotides comprises a 3′ blocking group;
(iii) imaging and performing one or more fluorescent measurements of the extended copy polynucleotides in an aqueous scan mixture to determine the identity of the incorporated nucleotides; and
(iv) removing the 3′ blocking groups and the detectable labels of the incorporated nucleotides;

wherein the aqueous scan mixture comprises one or more additives for reducing fluorescent signal decay caused by the fluorescent measurements, and wherein the one or more additives comprise a tri-substituted 1,4-cyclooctatetraene (COT) analog.

In some embodiments of the method described herein, the tri-substituted 1,4-COT analog has the structure of formula (VI):

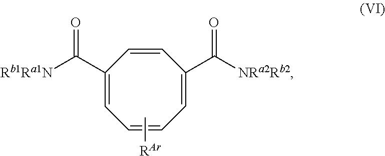

wherein
each of R^{a1}, R^{a2}, R^{b1} and R^{b2} is independently H, unsubstituted C₁-C₆ alkyl, or substituted C₁-C₆ alkyl;

R^{Ar} is carboxyl, C₁-C₆ alkyl, substituted C₁-C₆ alkyl, C₁-C₆ alkoxy, substituted C₁-C₆ alkoxy, C₁-C₆ haloalkyl, C₁-C₆ haloalkoxy, (C₁-C₆ alkoxy)C₁-C₆ alkyl, —O(C₁-C₆ alkoxy)C₁-C₆ alkyl, optionally substituted amino, amino(C₁-C₆ alkyl), halo, cyano, hydroxy, hydroxy(C₁-C₆ alkyl), nitro, sulfonyl, sulfo, sulfonate, S-sulfonamido, N-sulfonamido, C₆-C₁₀ aryl, 5 to 10 membered heteroaryl, C₃-C₁₀ carbocyclyl, 3 to 10 membered heterocyclyl, —C(═O)OR¹⁰, —C(═O)NR¹¹ R¹²; −(CH₂CH₂O)ₙ(C₁-C₆ alkyl), or —O(CH₂CH₂O)ₙ(C₁-C₆ alkyl); and
each of R¹⁰, R¹¹ and R¹² is independently H, unsubstituted C₁-C₆ alkyl, or substituted C₁-C₆ alkyl.

In some embodiments of the method described herein, the tri-substituted 1,4-COT analog has the structure of formula (VI-1):

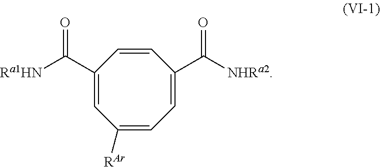

In some such embodiments, R^{Ar} is sulfo, chloro, bromo, nitro, phenyl, C₁-C₆ alkyl (such as methyl, ethyl, isopropyl, propyl, n-butyl, t-butyl), —NH(C₁-C₆ alkyl) (e.g., —NHCH₃ or NHCH₂CH₃), or —N(C₁-C₆ alkyl)₂ (e.g., —N(CH₃)₂, —N(CH₃)(CH₂CH₃) or —N(CH₂CH₃)₂). In some such embodiments, each of R^{a1} and R^{a2} is independently C₁-C₆ alkyl, unsubstituted or substituted with carboxyl, sulfo, or sulfonate.

In some embodiments of the method described herein, the scan mixture further comprises a radical scavenger (such as reactive oxygen species (ROS) scavenger), an oxygen scavenger (such as O₂ scavenger), a reducing reagent, an antioxidant, or combinations thereof. Cyclooctatetraene (COT) or a substituted analog thereof can act as a triplet state quencher (TSQ). In further embodiments, the scan mixture may further comprise one or more additional triplet state quencher (TSQ). Non-limiting example of additional TSQ includes a nickel (II) salt or complex, 2-mercaptoethylamine (MEA) or a salt thereof, 6-hydroxy-2,5,7,8-tetramethylchroman-2-carboxylic acid (Trolox) or a salt thereof, or combinations thereof. Non-limiting examples of the oxygen scavenger include an enzyme capable of reacting with oxygen, glucose oxidase, catalase, diethylhydroxylamine (DEHA), or hydroquinone, or combinations thereof. Non-limiting examples of the radical scavenger include 1,4-diazabicyclo[2.2.2]octane (DABCO), caffeine, mannitol, or combinations thereof. Non-limiting examples of the reducing reagent include a phosphine or a salt thereof, sodium sulfite (Na₂SO₃), a thiol containing compound, 2-mercaptoethanol (bME), cysteine or an analog thereof, and combinations thereof. For example, the phosphine may comprise tris(hydroxypropyl)phosphine (THP), tris(hydroxymethyl)phosphine (THMP), tris(carboxyethyl)phosphine (TCEP), bis(p-sulfonatophenyl)phenylphosphine dihydrate potassium salt, or triphenylphosphine-3,3′,3″-trisulfonic acid trisodium salt. Non-limiting example of an antioxidant include ascorbic acid or salts thereof (such as sodium ascorbate), 2-hydroxylethyl gallate (HEG), and gallic acid. In some embodiments, the aqueous scan mix may further comprise an ascorbate salt (e.g., sodium ascorbate). In other embodiments, the aqueous scan mix does not comprise an ascorbate salt (e.g., sodium ascorbate).

In some embodiments of the method described herein, the aqueous scan mixture may further comprise one or more buffering agents (e.g., Tris, glycine, MOPS, HEPES, etc.) or surfactants (e.g., Tween 20), or combinations thereof. In some embodiments, the aqueous scan mixture has a basic pH of from about 7.2 to about 8.0, for example, about 7.2, 7.3, 7.4, 7.5, 7.6, 7.7, 7.8, 7.9 or 8.0, or a range defined by any two of the preceding values.

In some embodiments of the method described herein, step (iii) comprising using two light sources operating at wavelengths between 450-460 nm and between 520-535 nm. In other embodiments, step (iii) comprising using a single source operating at wavelengths between 450-460 nm.

In some embodiments of the method described herein, the method further comprises step (v) contacting the solid support with an aqueous wash solution. In further embodiments, wherein steps (i) through (v) are repeated at least about 50, 100, 150, 200, 250, 300, 350, 400, 450 or 500 cycles to determine the target polynucleotides sequences.

### Methods of Sequencing

Nucleotides labeled with a photo-protected florescent compound according to the present disclosure may be used in any method of analysis such as method that include detection of a fluorescent label attached to such nucleotide, whether on its own or incorporated into or associated with a larger molecular structure or conjugate. In this context the term “incorporated into a polynucleotide” can mean that the 5′ phosphate is joined in phosphodiester linkage to the 3′ hydroxyl group of a second nucleotide, which may itself form part of a longer polynucleotide chain. The 3′ end of a nucleotide set forth herein may or may not be joined in phosphodiester linkage to the 5′ phosphate of a further nucleotide. Thus, in one non-limiting embodiment, the disclosure provides a method of detecting a labeled nucleotide incorporated into a polynucleotide which comprises: (a) incorporating at least one labeled nucleotide of the disclosure into a polynucleotide and (b) determining the identity of the nucleotide(s) incorporated into the polynucleotide by detecting the fluorescent signal from the fluorescent compound attached to said nucleotide(s). One particular embodiment of the method of sequencing utilizes a two-excitation, two-channel detection system (also known as 2Ex-2Ch), which includes a blue excitation light source having a wavelength between about 450 nm to about 460 nm, and a second green excitation light source having a wavelength between about 520 nm to 540 nm, or about 532 nm. Another embodiment of the method of sequencing utilizes a one-excitation, two-channel detection system (also known as 1Ex-2Ch), which contain a blue excitation light source having a wavelength between about 450 nm to about 460 nm, and two separate collection channels/filters at both the blue and green regions (e.g., at a blue region with a wavelength ranging from about 472 to about 520 nm, and at a green region with a wavelength ranging from about 540 nm to about 640nm). Another particular embodiment of the method of sequencing utilizes a two-excitation, one channel detection system (also known as 2Ex-1Ch), which includes a green excitation light at about 520 nm to about 530 nm and a blue excitation light at about 450 nm to about 460 nm.

The sequencing method can include: a synthetic step (a) in which one or more labeled nucleotides according to the disclosure are incorporated into a polynucleotide and a detection step (b) in which one or more labeled nucleotide(s) incorporated into the polynucleotide are detected by detecting or quantitatively measuring their fluorescence.

Some embodiments of the present application are directed to a method for determining the sequences of a plurality of different target polynucleotides, comprising:
(a) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(b) contacting the solid support with an aqueous solution comprising DNA polymerase and one more of four different types of nucleotides (e.g., dATP, dGTP, dCTP and dTTP or dUTP), under conditions suitable for DNA polymerase-mediated primer extension, and incorporating one type of nucleotides into the sequencing primers to produce extended copy polynucleotides, wherein at least one type of nucleotide is a labeled nucleotide containing the photo-protected fluorescent compound described herein, and wherein each of the four types of nucleotides comprises a 3′ blocking group;
(c) imaging the solid support and performing one or more fluorescent measurements of the extended copy polynucleotides; and
(d) removing the 3′ blocking group of the incorporated nucleotides. In some embodiments, step (d) also removes the labels of the incorporated nucleotides (if the incorporated nucleotides are labeled). In some such embodiments, the labels and the 3′ blocking groups of the incorporated nucleotides are removed in a single chemical reaction. In some further embodiments, the method may also comprises
(e) washing the solid support with an aqueous wash solution (e.g., washing the removed label moiety and the 3′ blocking group away from the extended copy polynucleotides). In some embodiments, steps (b) through (e) are repeated at least 30, 40, 50, 60, 70, 80, 90, 100, 110, 120, 130, 140, 150, 160, 170, 180, 190, 200, 250, 300, 350, 400, 450 or 500 cycles to determine the target polynucleotide sequences. In some embodiments, the four types of nucleotides comprise dATP, dCTP, dGTP and dTTP or dUTP, or non-natural nucleotide analogs thereof. In some embodiments, the sequence determination is conducted after the completion of repeated cycles of the sequencing steps described herein. In some embodiments, the photo-protected fluorescent compound may be used as any one of the first, the second or the third label described in the method.

In some further embodiments, the method is performed on an automated sequencing instrument, and wherein the automated sequencing instrument comprises a single light source operating with a blue laser at about 450 nm to about 460 nm. In other embodiments, the automatic sequencing instrument may comprise two light sources operating at different wavelengths (e.g., at about 450-460 nm and about 520-530 nm).

In some embodiments, at least one nucleotide is incorporated into a polynucleotide (such as a single stranded primer polynucleotide described herein) in the synthetic step by the action of a polymerase enzyme. However, other methods of joining nucleotides to polynucleotides, such as, for example, chemical oligonucleotide synthesis or ligation of labeled oligonucleotides to unlabeled oligonucleotides, can be used. Therefore, the term “incorporating,” when used in reference to a nucleotide and polynucleotide, can encompass polynucleotide synthesis by chemical methods as well as enzymatic methods.

In a specific embodiment, a synthetic step is carried out and may optionally comprise incubating a template or target polynucleotide strand with a reaction mixture comprising fluorescently labeled nucleotides of the disclosure. A polymerase can also be provided under conditions which permit formation of a phosphodiester linkage between a free 3′ hydroxyl group on a polynucleotide strand annealed to the template or target polynucleotide strand and a 5′ phosphate group on the labeled nucleotide. Thus, a synthetic step can include formation of a polynucleotide strand as directed by complementary base pairing of nucleotides to a template/target strand.

In all embodiments of the methods, the detection step may be carried out while the polynucleotide strand into which the labeled nucleotides are incorporated is annealed to a template/target strand, or after a denaturation step in which the two strands are separated. Further steps, for example chemical or enzymatic reaction steps or purification steps, may be included between the synthetic step and the detection step. In particular, the polynucleotide strand incorporating the labeled nucleotide(s) may be isolated or purified and then processed further or used in a subsequent analysis. By way of example, polynucleotide strand incorporating the labeled nucleotide(s) as described herein in a synthetic step may be subsequently used as labeled probes or primers. In other embodiments, the product of the synthetic step set forth herein may be subject to further reaction steps and, if desired, the product of these subsequent steps purified or isolated.

Suitable conditions for the synthetic step will be well known to those familiar with standard molecular biology techniques. In one embodiment, a synthetic step may be analogous to a standard primer extension reaction using nucleotide precursors, including the labeled nucleotides as described herein, to form an extended polynucleotide strand (primer polynucleotide strand) complementary to the template/target strand in the presence of a suitable polymerase enzyme. In other embodiments, the synthetic step may itself form part of an amplification reaction producing a labeled double stranded amplification product comprised of annealed complementary strands derived from copying of the primer and template polynucleotide strands. Other exemplary synthetic steps include nick translation, strand displacement polymerization, random primed DNA labeling, etc. A particularly useful polymerase enzyme for a synthetic step is one that is capable of catalyzing the incorporation of the labeled nucleotides as set forth herein. A variety of naturally occurring or mutant/modified polymerases can be used. By way of example, a thermostable polymerase can be used for a synthetic reaction that is carried out using thermocycling conditions, whereas a thermostable polymerase may not be desired for isothermal primer extension reactions. Suitable thermostable polymerases which are capable of incorporating the labeled nucleotides according to the disclosure include those described in WO 2005/024010 or WO06120433, each of which is incorporated herein by reference. In synthetic reactions which are carried out at lower temperatures such as 37° C., polymerase enzymes need not necessarily be thermostable polymerases, therefore the choice of polymerase will depend on a number of factors such as reaction temperature, pH, strand-displacing activity and the like.

In specific non-limiting embodiments, the disclosure encompasses methods of nucleic acid sequencing, re-sequencing, whole genome sequencing, single nucleotide polymorphism scoring, any other application involving the detection of the modified nucleotide or nucleoside labeled with photo-protected fluorescent compounds described herein when incorporated into a polynucleotide.

A particular embodiment of the disclosure provides use of labeled nucleotides according to the disclosure in a polynucleotide sequencing-by-synthesis reaction. Sequencing-by-synthesis generally involves sequential addition of one or more nucleotides or oligonucleotides to a growing polynucleotide chain in the 5′ to 3′ direction using a polymerase or ligase in order to form an extended polynucleotide chain complementary to the template/target nucleic acid to be sequenced. The identity of the base present in one or more of the added nucleotide(s) can be determined in a detection or “imaging” step. The identity of the added base may be determined after each nucleotide incorporation step. The sequence of the template may then be inferred using conventional Watson-Crick base-pairing rules. The use of the nucleotides labeled with photo-protected fluorescent compounds set forth herein for determination of the identity of a single base may be useful, for example, in the scoring of single nucleotide polymorphisms, and such single base extension reactions are within the scope of this disclosure.

In an embodiment of the present disclosure, the sequence of a template/target polynucleotide is determined by detecting the incorporation of one or more nucleotides into a nascent strand complementary to the template polynucleotide to be sequenced through the detection of fluorescent label(s) attached to the incorporated nucleotide(s). Sequencing of the template polynucleotide can be primed with a suitable primer (or prepared as a hairpin construct which will contain the primer as part of the hairpin), and the nascent chain is extended in a stepwise manner by addition of nucleotides to the 3′ end of the primer in a polymerase-catalyzed reaction.

In particular embodiments, each of the different nucleotide triphosphates (A, T, G and C) may be labeled with a unique fluorophore and also comprises a blocking group at the 3′ position to prevent uncontrolled polymerization. Alternatively, one of the four nucleotides may be unlabeled (dark). The polymerase enzyme incorporates a nucleotide into the nascent chain complementary to the template/target polynucleotide, and the blocking group prevents further incorporation of nucleotides. Any unincorporated nucleotides can be washed away and the fluorescent signal from each incorporated nucleotide can be “read” optically by suitable means, such as a charge-coupled device using light source excitation and suitable emission filters. The 3′ blocking group and fluorescent compounds can then be removed (deprotected) (simultaneously or sequentially) to expose the nascent chain for further nucleotide incorporation. Typically, the identity of the incorporated nucleotide will be determined after each incorporation step, but this is not strictly essential. Similarly, U.S. Pat. No. 5,302,509 (which is incorporated herein by reference) discloses a method to sequence polynucleotides immobilized on a solid support.

The method, as exemplified above, utilizes the incorporation of fluorescently labeled, 3′-blocked nucleotides A, G, C, and T into a growing strand complementary to the immobilized polynucleotide, in the presence of DNA polymerase. The polymerase incorporates a base complementary to the target polynucleotide but is prevented from further addition by the 3′-blocking group. The label of the incorporated nucleotide can then be determined, and the blocking group removed by chemical cleavage to allow further polymerization to occur. The nucleic acid template to be sequenced in a sequencing-by-synthesis reaction may be any polynucleotide that it is desired to sequence. The nucleic acid template for a sequencing reaction will typically comprise a double stranded region having a free 3′ hydroxyl group that serves as a primer or initiation point for the addition of further nucleotides in the sequencing reaction. The region of the template to be sequenced will overhang this free 3′ hydroxyl group on the complementary strand. The overhanging region of the template to be sequenced may be single stranded but can be double-stranded, provided that a “nick is present” on the strand complementary to the template strand to be sequenced to provide a free 3′ OH group for initiation of the sequencing reaction. In such embodiments, sequencing may proceed by strand displacement. In certain embodiments, a primer bearing the free 3′ hydroxyl group may be added as a separate component (e.g., a short oligonucleotide) that hybridizes to a single-stranded region of the template to be sequenced. Alternatively, the primer and the template strand to be sequenced may each form part of a partially self-complementary nucleic acid strand capable of forming an intra-molecular duplex, such as for example a hairpin loop structure. Hairpin polynucleotides and methods by which they may be attached to solid supports are disclosed in PCT Publication Nos. WO 01/57248 and WO2005/047301, each of which is incorporated herein by reference. Nucleotides can be added successively to a growing primer, resulting in synthesis of a polynucleotide chain in the 5′ to 3′ direction. The nature of the base which has been added may be determined, particularly but not necessarily after each nucleotide addition, thus providing sequence information for the nucleic acid template. Thus, a nucleotide is incorporated into a nucleic acid strand (or polynucleotide) by joining of the nucleotide to the free 3′ hydroxyl group of the nucleic acid strand via formation of a phosphodiester linkage with the 5′ phosphate group of the nucleotide.

The nucleic acid template to be sequenced may be DNA or RNA, or even a hybrid molecule comprised of deoxynucleotides and ribonucleotides. The nucleic acid template may comprise naturally occurring and/or non-naturally occurring nucleotides and natural or non-natural backbone linkages, provided that these do not prevent copying of the template in the sequencing reaction.

In certain embodiments, the nucleic acid template to be sequenced may be attached to a solid support via any suitable linkage method known in the art, for example via covalent attachment. In certain embodiments template polynucleotides may be attached directly to a solid support (e.g., a silica-based support). However, in other embodiments of the disclosure the surface of the solid support may be modified in some way so as to allow either direct covalent attachment of template polynucleotides, or to immobilize the template polynucleotides through a hydrogel or polyelectrolyte multilayer, which may itself be non-covalently attached to the solid support.

Arrays in which polynucleotides have been directly attached to a support (for example, silica-based supports such as those disclosed in WO 00/06770 (incorporated herein by reference), wherein polynucleotides are immobilized on a glass support by reaction between a pendant epoxide group on the glass with an internal amino group on the polynucleotide. In addition, polynucleotides can be attached to a solid support by reaction of a sulfur-based nucleophile with the solid support, for example, as described in WO 2005/047301 (incorporated herein by reference). A still further example of solid-supported template polynucleotides is where the template polynucleotides are attached to hydrogel supported upon silica-based or other solid supports, for example, as described in WO 00/31148, WO 01/01143, WO 02/12566, WO 03/014392, U.S. Pat. No. 6,465,178 and WO 00/53812, each of which is incorporated herein by reference.

A particular surface to which template polynucleotides may be immobilized is a polyacrylamide hydrogel. Polyacrylamide hydrogels are described in the references cited above and in WO 2005/065814, which is incorporated herein by reference. Specific hydrogels that may be used include those described in WO 2005/065814 and U.S. Pub. No. 2014/0079923. In one embodiment, the hydrogel is PAZAM (poly(N-(5-azidoacetamidylpentyl) acrylamide-co-acrylamide)).

DNA template molecules can be attached to beads or microparticles, for example, as described in U.S. Pat. No. 6,172,218 (which is incorporated herein by reference). Attachment to beads or microparticles can be useful for sequencing applications. Bead libraries can be prepared where each bead contains different DNA sequences. Exemplary libraries and methods for their creation are described in Nature, 437, 376-380 (2005); Science, 309, 5741, 1728-1732 (2005), each of which is incorporated herein by reference. Sequencing of arrays of such beads using nucleotides set forth herein is within the scope of the disclosure.

Template(s) that are to be sequenced may form part of an “array” on a solid support, in which case the array may take any convenient form. Thus, the method of the disclosure is applicable to all types of high-density arrays, including single-molecule arrays, clustered arrays, and bead arrays. Nucleotides labeled with photo-protected fluorescent compounds of the present disclosure may be used for sequencing templates on essentially any type of array, including but not limited to those formed by immobilization of nucleic acid molecules on a solid support.

However, nucleotides labeled with photo-protected fluorescent compounds of the disclosure are particularly advantageous in the context of sequencing of clustered arrays. In clustered arrays, distinct regions on the array (often referred to as sites, or features) comprise multiple polynucleotide template molecules. Generally, the multiple polynucleotide molecules are not individually resolvable by optical means and are instead detected as an ensemble. Depending on how the array is formed, each site on the array may comprise multiple copies of one individual polynucleotide molecule (e.g., the site is homogenous for a particular single- or double-stranded nucleic acid species) or even multiple copies of a small number of different polynucleotide molecules (e.g., multiple copies of two different nucleic acid species). Clustered arrays of nucleic acid molecules may be produced using techniques generally known in the art. By way of example, WO 98/44151 and WO 00/18957, each of which is incorporated herein, describe methods of amplification of nucleic acids wherein both the template and amplification products remain immobilized on a solid support in order to form arrays comprised of clusters or “colonies” of immobilized nucleic acid molecules. The nucleic acid molecules present on the clustered arrays prepared according to these methods are suitable templates for sequencing using nucleotides labeled with the photo-protected fluorescent compounds of the disclosure.

Nucleotides labeled with the photo-protected fluorescent compounds of the present disclosure are also useful in sequencing of templates on single molecule arrays. The term “single molecule array” or “SMA” as used herein refers to a population of polynucleotide molecules, distributed (or arrayed) over a solid support, wherein the spacing of any individual polynucleotide from all others of the population is such that it is possible to individually resolve the individual polynucleotide molecules. The target nucleic acid molecules immobilized onto the surface of the solid support can thus be capable of being resolved by optical means in some embodiments. This means that one or more distinct signals, each representing one polynucleotide, will occur within the resolvable area of the particular imaging device used.

Single molecule detection may be achieved wherein the spacing between adjacent polynucleotide molecules on an array is at least 100 nm, more particularly at least 250 nm, still more particularly at least 300 nm, even more particularly at least 350 nm. Thus, each molecule is individually resolvable and detectable as a single molecule fluorescent point, and fluorescence from said single molecule fluorescent point also exhibits single step photobleaching.

The terms “individually resolved” and “individual resolution” are used herein to specify that, when visualized, it is possible to distinguish one molecule on the array from its neighboring molecules. Separation between individual molecules on the array will be determined, in part, by the particular technique used to resolve the individual molecules. The general features of single molecule arrays will be understood by reference to published applications WO 00/06770 and WO 01/57248, each of which is incorporated herein by reference. Although one use of the labeled nucleotides of the disclosure is in sequencing-by-synthesis reactions, the utility of such nucleotides is not limited to such methods. In fact, the labeled nucleotides described herein may be used advantageously in any sequencing methodology which requires detection of fluorescent labels attached to nucleotides incorporated into a polynucleotide.

In particular, nucleotides labeled with fluorescent compounds of the disclosure may be used in automated fluorescent sequencing protocols, particularly fluorescent dye-terminator cycle sequencing based on the chain termination sequencing method of Sanger and co-workers. Such methods generally use enzymes and cycle sequencing to incorporate fluorescently labeled dideoxynucleotides in a primer extension sequencing reaction. So-called Sanger sequencing methods, and related protocols (Sanger-type), utilize randomized chain termination with labeled dideoxynucleotides.

Thus, the present disclosure also encompasses nucleotides labeled with fluorescent compounds which are dideoxynucleotides lacking hydroxyl groups at both of the 3′ and 2′ positions, such modified dideoxynucleotides being suitable for use in Sanger type sequencing methods and the like.

Nucleotides labeled with photo-protected fluorescent compounds of the present disclosure incorporating 3′ blocking groups, it will be recognized, may also be of utility in Sanger methods and related protocols since the same effect achieved by using dideoxy nucleotides may be achieved by using nucleotides having 3′ OH blocking groups: both prevent incorporation of subsequent nucleotides. Where nucleotides according to the present disclosure and having a 3′ blocking group are to be used in Sanger-type sequencing methods it will be appreciated that the fluorescent compounds or detectable labels attached to the nucleotides need not be connected via cleavable linkers, since in each instance where a labeled nucleotide of the disclosure is incorporated; no nucleotides need to be subsequently incorporated and thus the label need not be removed from the nucleotide.

Alternatively, the sequencing methods described herein may also be carried out using unlabeled nucleotides and affinity reagents containing a fluorescent compound described herein. For example, one, two, three or each of the four different types of nucleotides (e.g., dATP, dCTP, dGTP and dTTP or dUTP) in the incorporation mixture of step (a) may be unlabeled. Each of the four types of nucleotides (e.g., dNTPs) has a 3′ blocking group to ensure that only a single base can be added by a polymerase to the 3′ end of the primer polynucleotide. After incorporation of an unlabeled nucleotide in step (b), the remaining unincorporated nucleotides are washed away. An affinity reagent is then introduced that specifically recognizes and binds to the incorporated dNTP to provide a labeled extension product comprising the incorporated dNTP. Uses of unlabeled nucleotides and affinity reagents in sequencing by synthesis have been disclosed in WO 2018/129214 and WO 2020/097607. A modified sequencing method of the present disclosure using unlabeled nucleotides may include the following steps:
(a′) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(b′) contacting the solid support with an aqueous solution comprising DNA polymerase and one more of four different types of unlabeled nucleotides (e.g., dATP, dCTP, dGTP, and dTTP or dUTP) under conditions suitable for DNA polymerase-mediated primer extension, and incorporating one type of nucleotides into the sequencing primers to produce extended copy polynucleotides, and wherein each of the four types of nucleotides comprises a 3′ blocking group;
(c′) contacting the extended copy polynucleotides with a set of affinity reagents under conditions wherein one affinity reagent binds specifically to the incorporated unlabeled nucleotides to provide labeled extended copy polynucleotides;

(d′) imaging the solid support and performing one or more fluorescent measurements of the extended copy polynucleotides; and
(e′) removing the 3′ blocking group of the incorporated nucleotides.

In some embodiments of the modified sequencing method described herein, the method further comprises removing the affinity reagents from the incorporated nucleotides. In still further embodiments, the 3′ blocking group and the affinity reagent are removed in the same reaction. In some embodiments, the method further comprises a step (f′) washing the solid support with an aqueous wash solution. In further embodiments, steps (b′) through (f′) are repeated at least 50, 100, 150, 200, 250, 300, 350, 400, 450 or 500 cycles to determine the target polynucleotide sequences. In some embodiments, the set of affinity reagents may comprise a first affinity reagent that binds specifically to the first type of nucleotide, a second affinity reagent that binds specifically to the second type of nucleotide, and a third affinity reagent that binds specifically to the third type of nucleotide. In some further embodiments, each of the first, second and the third affinity reagents comprises a detectable label that is spectrally distinguishable. In some embodiments, the affinity reagents may include protein tags, antibodies (including but not limited to binding fragments of antibodies, single chain antibodies, bispecific antibodies, and the like), aptamers, knottins, affimers, or any other known agent that binds an incorporated nucleotide with a suitable specificity and affinity. In one embodiment, at least one affinity reagent is an antibody or a protein tag. In another embodiment, at least one of the first type, the second type, and the third type of affinity reagents is an antibody or a protein tag comprising one or more detectable labels (e.g., multiple copies of the same detectable label), wherein the detectable label is or comprises a photo-protected fluorescent compound described herein.

### EXAMPLES

Additional embodiments are disclosed in further detail in the following examples, which are not in any way intended to limit the scope of the claims.

### Example 1. Synthesis of Fluorescent Compounds with Photoprotective Moieties

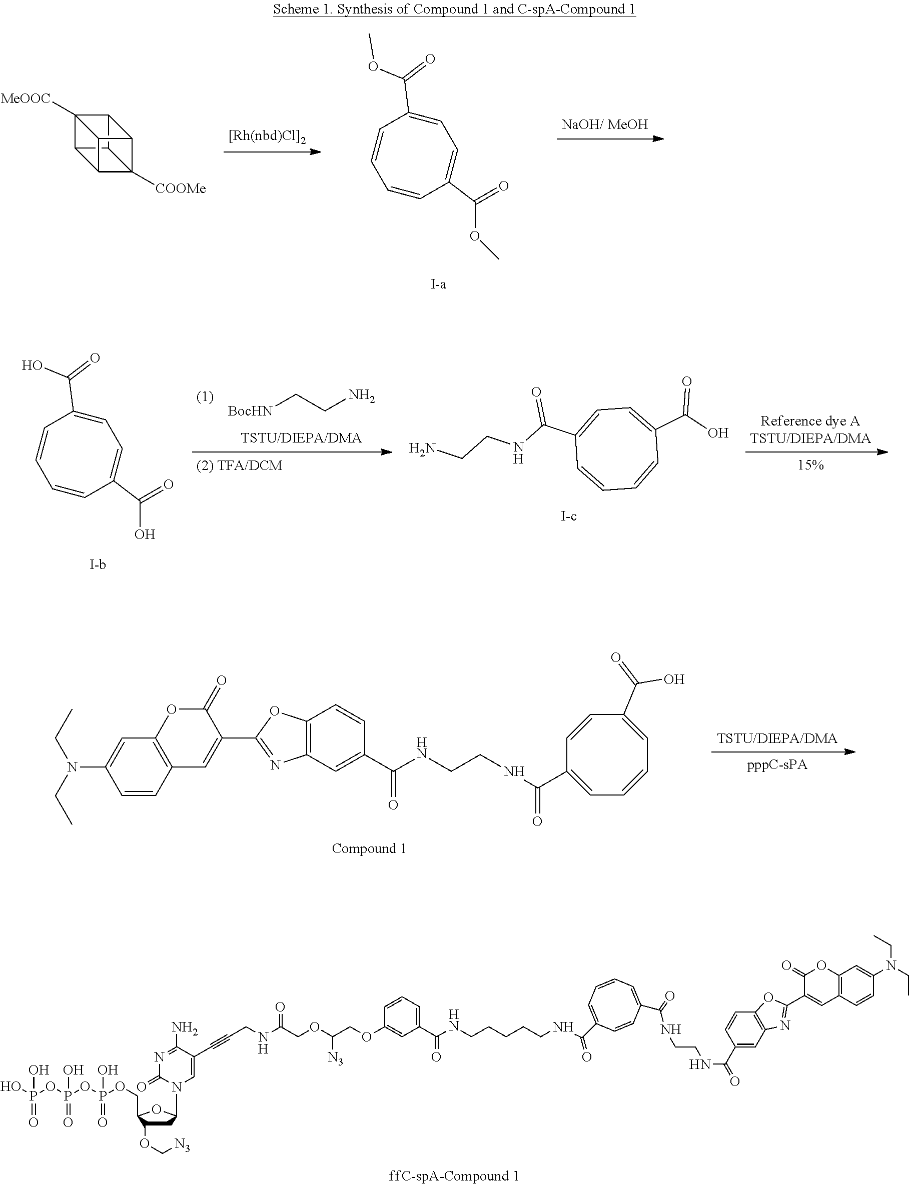

Synthesis of dimethyl-cyclooctatetraene-1,4-dicarboxylate (1-a). Dimethyl cubane-1,4-dicarboxylate (1 eq, 2.273mmol, 0.500g) and bicyclo[2.2.1]heptan-2,5-diene-rhodium(I) chloride dimer (0.1 eq, 0.2273 mmol, 0.106 g) were dissolved in toluene (30 mL) and heated at 60 ° C. for 16 h and purified by column chromatography (20% ethyl acetate/petroleum ether v/v) gave 1-a (0.370 g, 64%) as a yellow solid. ¹H-NMR (600 MHz, CDCl3): δ (ppm) 7.10-7.03 (m, 2H), 6.17 (s, 1H), 6.09-5.99 (m, 3H), 3.77-3.74 (m, 6H).

Synthesis of cyclooctatetraene-1,4-dicarboxylic acid (1-b). To a solution of I-a (0.370 g, 1.68 mmol) in methanol (30 mL) was added a solution of sodium hydroxide (0.592 g, 14.8 mmol) in methanol (15 mL). The solution was left to stir for 16 h at 40° C. then the methanol was removed in vacuo. The residue was suspended in water (25 mL) then washed with DCM (3×50 mL). The aqueous phase was acidified to pH 1 with HCl (3M, 10 mL) to give the title compound 1-b (0.285 mg, 88.17%) as a yellow solid which was collected by filtration. ¹H-NMR (600 MHz, DMSO-d₆): δ (ppm) 6.99-6.91 (m, 2H), 6.09-5.95 (m, 4H).

Synthesis of Compound 1-c. Cyclooctatetraene-1,4-dicarboxylic acid (1-b) (198.0 mg, 1000 umol) was dissolved in DMF (anhydrous, 1 mL) and evaporated to dry. The procedure was repeated for 3 times. Anhydrous DMF (5 mL) and Hunig's base (1290 mL, 10000 lima 10 equivalents) was then pipetted into the round bottom flask. TSTU (301.0 mg, 1000 umol, 1.0 equivalents) was added in one portion. The reaction mixture was kept at room temperature. After 30 min, N-Boc-ethylenediamine (145 mg, 900 μmol, 0.9 equivalents) in 0.1 M TEAB was added to the reaction mixture and stirred at room temperature for 3 h. The reaction was quenched with TEAB buffer (0.1 M, 10 ml) and the volatile solvent was removed by reduced pressure evaporation (HV) and crude was used in the next step without further purification. Above crude 1-c was dissolved in 10 mL of 15% TFA in DCM and reacted at room temperature for 3 hrs and reaction was monitored by LC-MS. After reaction was completed, volatiles were removed by reduced pressure and dissolved in the 5 mL 0.1 M TEAB and purified by reverse phase C₁₈ column using 0.1 M TEAA buffer and ACN as the solvent. LC-MS [M+H]⁺235.24 (Yield=39.8%)

Synthesis of Compound 1. In a 25 ml round-bottom flask, reference dye A (39.3 mg,103.9 μmol, 1 eq) was dissolved in DMF (anhydrous, 1 mL) and evaporated to dry. The procedure was repeated for 3 times. Anhydrous DMA (3 mL) and Hunig's base (134 mg, 1039 μmol, 10 equivalents) was then pipetted into the round bottom flask. TSTU (34.3 mg, 114.3 umol, 1.1 equivalents) was added in one portion. The reaction mixture was kept at room temperature. After 30 min, TLC (CH3CN: H2O 85:15) analysis indicated that the reaction completed. Compound 1-c (73 mg, 311.6 μmol, 3 equivalents) in 0.1 M TEAB was added to the reaction mixture and stirred at room temperature for 16 h. The reaction was quenched with TEAB buffer (0.1 M, 10 ml) and the volatile solvent was removed by reduced pressure evaporation (HV) and purified by preparative HPLC. LC-MS [M-H]⁻ 593.42 (Yield=69.4%)

Synthesis of C-sPA-Compound 1. In a 25 ml round-bottom flask, Compound 1 (3.87 umol) was dissolved in DMF (anhydrous, 1 mL) and evaporated to dry. The procedure was repeated for 3 times. Anhydrous DMA (1.5 mL), DIEPA (4.9 mg, 10 eq, 38.7 μmol) and TSTU (2.3 mg, 7.74 μmol, 2 equivalents) were then added into the round bottom flask. The reaction mixture was kept at room temperature. After 30 min, TLC (CH3CN: H2O 8:2) analysis indicated that the reaction completed. Then a solution of pppC-sPA (15.48 μmol in 0.150 ml 0.1 M TEAB, 4 equivalent) and Et₃N (5 ul) was added to the reaction mixture and stirred at room temperature overnight. The reaction was quenched with TEAB buffer (0.1 M, 10 ml) and loaded on a DEAE Sephadex column (25 g Biotage column) with solvent system of 0.1 M TEAB and 1 M TEAB. The product was collected and passed through C₁₈ column to remove TEAB and purified by preparative HPLC to yield C-sPA-Compound 1. LC-MS [M-H]⁻1497.28 (Yield=77.5%).

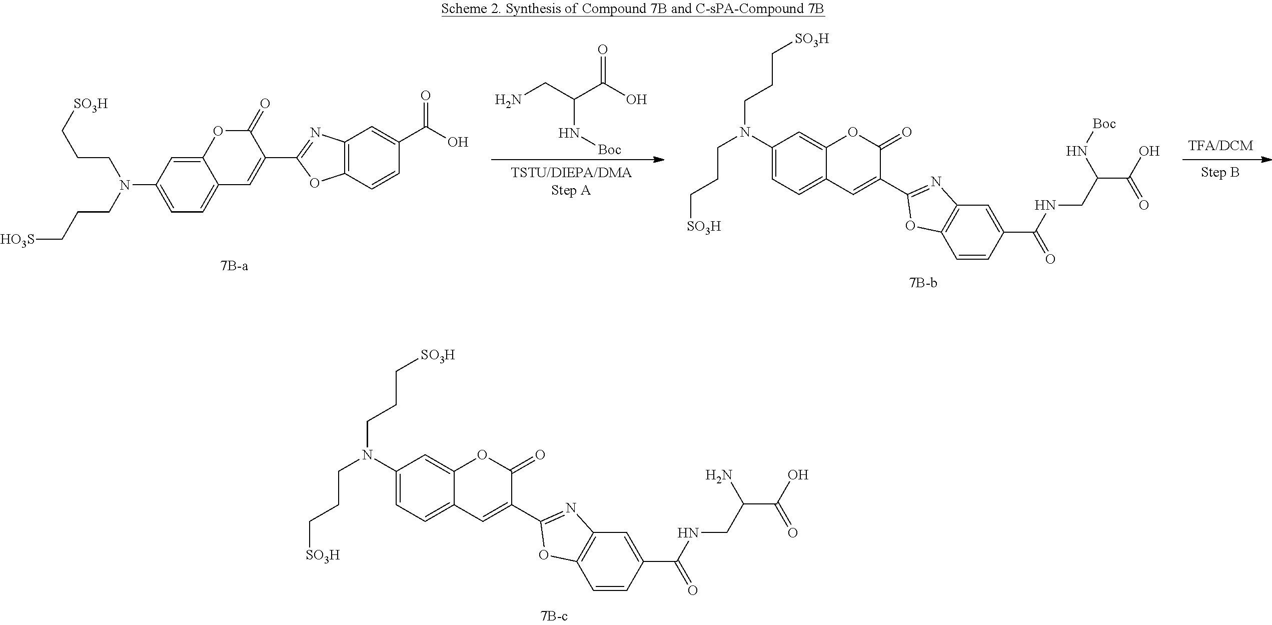 [SMILES: O=C(O)c1ccc2oc(-c3cc4ccc(N(CCCS(=O)(=O)O)CCCS(=O)(=O)O)cc4oc3=O)nc2c1 ; CC(C)(C)OC(=O)NC(CN)C(=O)O ; CC(C)(C)OC(=O)NC(CNC(=O)c1ccc2oc(-c3cc4ccc(N(CCCS(=O)(=O)O)CCCS(=O)(=O)O)cc4oc3=O)nc2c1)C(=O)O ; NC(CNC(=O)c1ccc2oc(-c3cc4ccc(N(CCCS(=O)(=O)O)CCCS(=O)(=O)O)cc4oc3=O)nc2c1)C(=O)O]

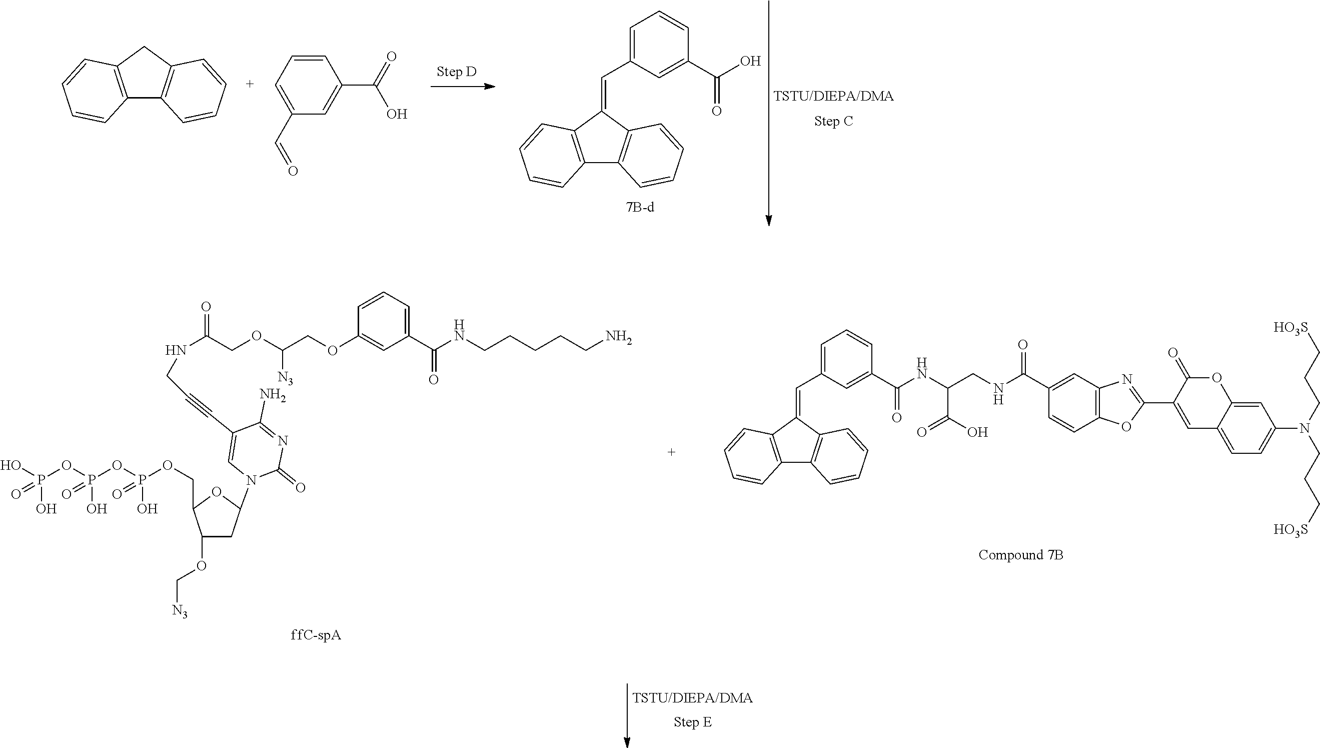

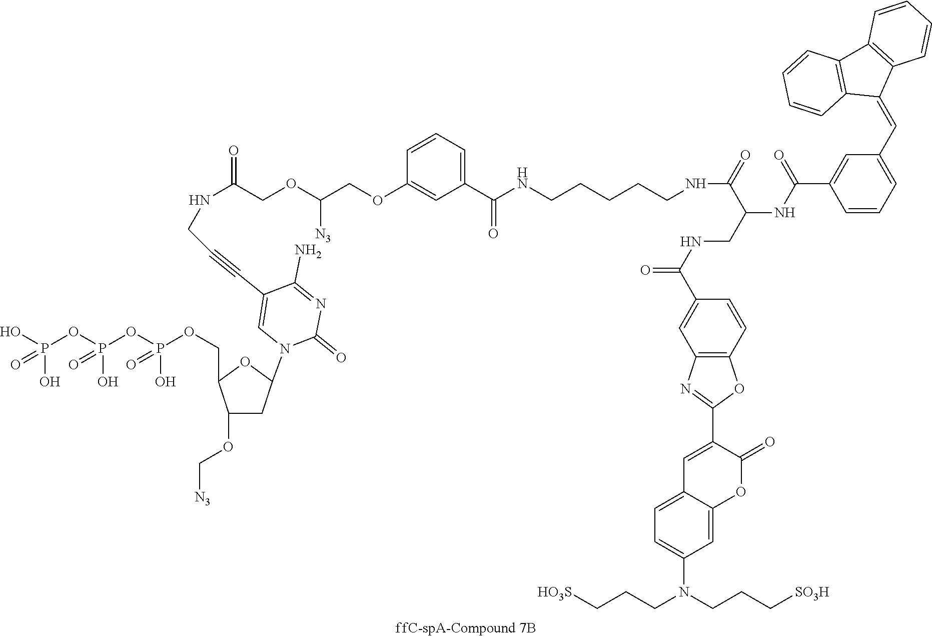 [SMILES: [N-]=[N+]=NCOC1CC(n2cc(C#CCNC(=O)COC(COc3cccc(C(=O)NCCCCCNC(=O)C(CNC(=O)c4ccc5oc(-c6cc7ccc(N(CCCS(=O)(=O)O)CCCS(=O)(=O)O)cc7oc6=O)nc5c4)NC(=O)c4cccc(C=C5c6ccccc6-c6ccccc65)c4)c3)N=[N+]=[N-])c(N)nc2=O)OC1COP(=O)(O)OP(=O)(O)OP(=O)(O)O]

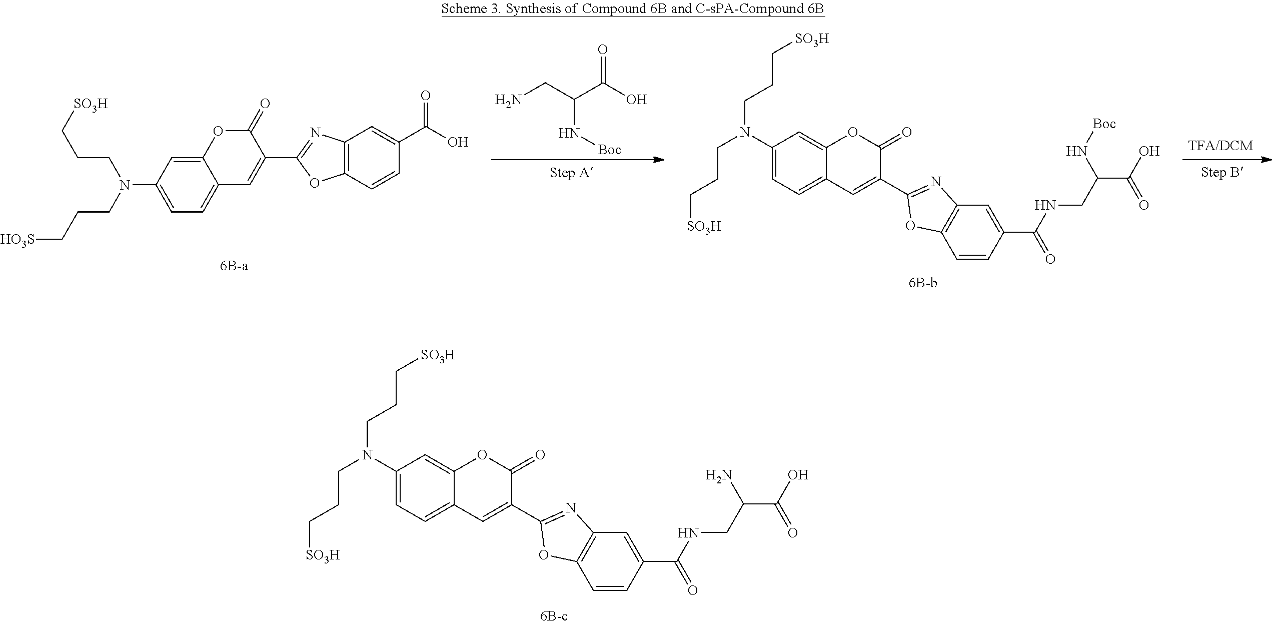 [SMILES: O=C(O)c1ccc2oc(-c3cc4ccc(N(CCCS(=O)(=O)O)CCCS(=O)(=O)O)cc4oc3=O)nc2c1 ; CC(C)(C)OC(=O)NC(CN)C(=O)O ; CC(C)(C)OC(=O)NC(CNC(=O)c1ccc2oc(-c3cc4ccc(N(CCCS(=O)(=O)O)CCCS(=O)(=O)O)cc4oc3=O)nc2c1)C(=O)O ; NC(CNC(=O)c1ccc2oc(-c3cc4ccc(N(CCCS(=O)(=O)O)CCCS(=O)(=O)O)cc4oc3=O)nc2c1)C(=O)O]

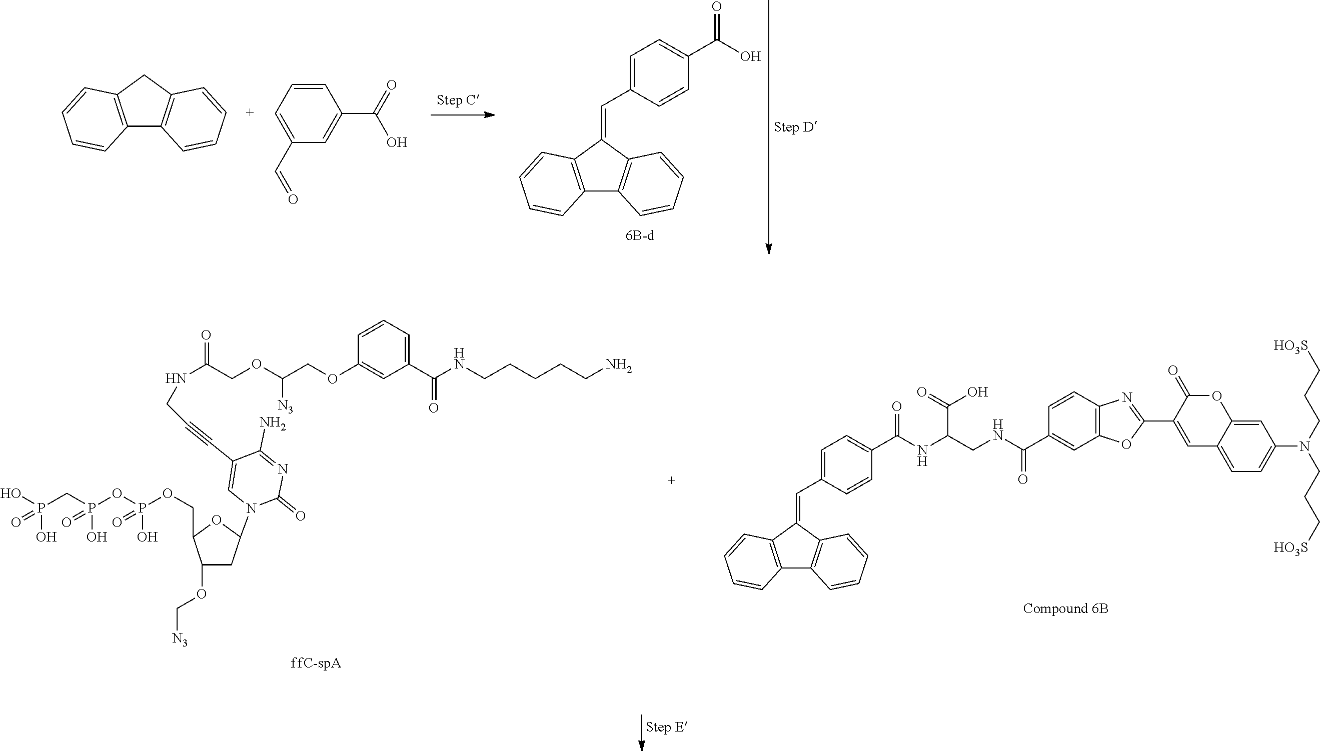

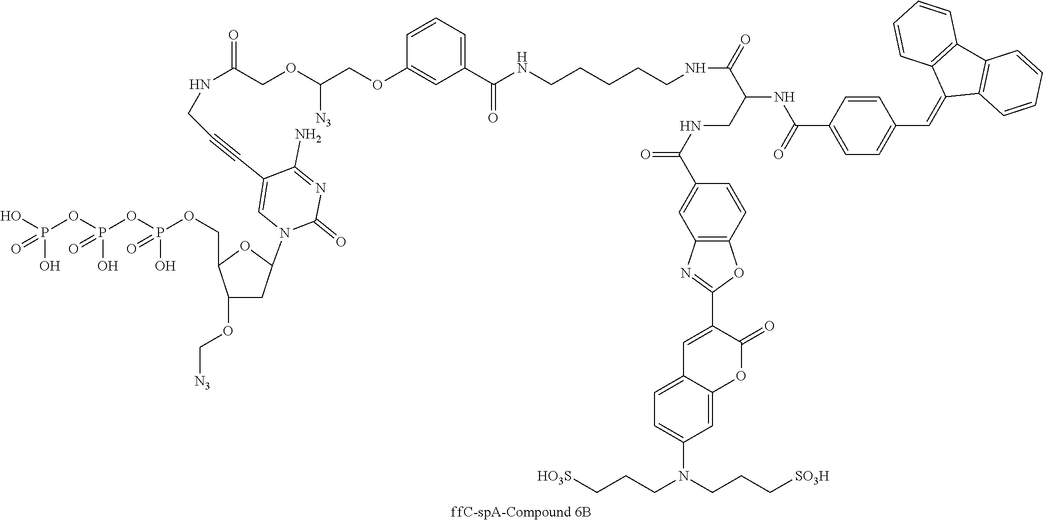 [SMILES: [N-]=[N+]=NCOC1CC(n2cc(C#CCNC(=O)COC(COc3cccc(C(=O)NCCCCCNC(=O)C(CNC(=O)c4ccc5oc(-c6cc7ccc(N(CCCS(=O)(=O)O)CCCS(=O)(=O)O)cc7oc6=O)nc5c4)NC(=O)c4ccc(C=C5c6ccccc6-c6ccccc65)cc4)c3)N=[N+]=[N-])c(N)nc2=O)OC1COP(=O)(O)OP(=O)(O)OP(=O)(O)O]

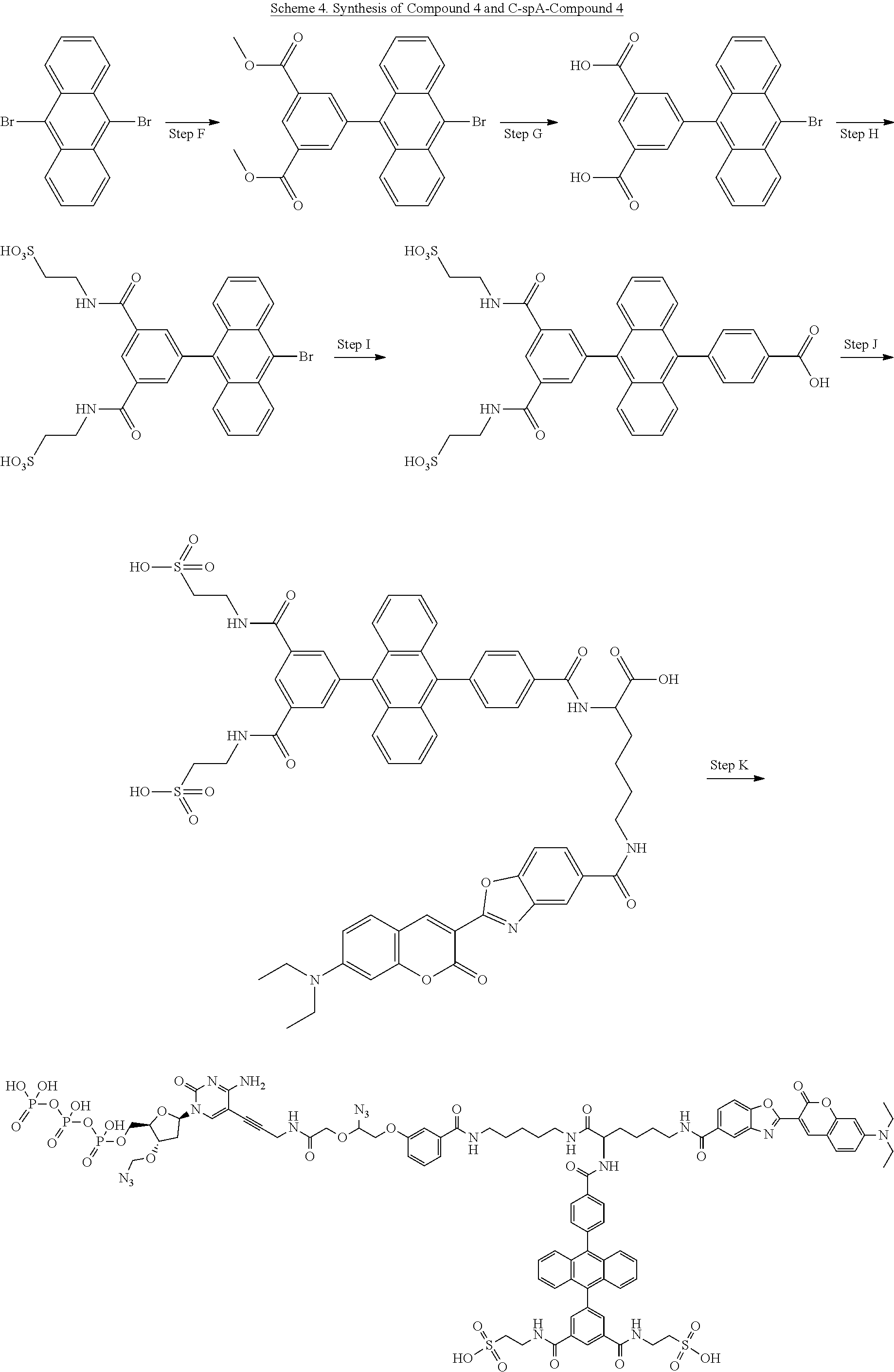

### General Procedure for Amide Coupling: (For steps A, C, E, A′, B′, E′, H, J and K)

In a round-bottom flask, acid (1 eq) was dissolved in DMF (anhydrous, 1 mL) and evaporated to dry. The procedure was repeated for 3 times. Anhydrous DMA and Hunig's base (10 equivalents) were then pipetted into the round bottom flask. Coupling agent TSTU (1.3 equivalents) was added in one portion. The reaction mixture was kept at room temperature. After 30 min, TLC (CH3CN: H2O 85:15) analysis indicated that the reaction completed. Amine (3 eq) in 0.1 M TEAB was added to the reaction mixture and stirred at room temperature for 16 h. TLC (CH3CN: H2O 8:2) showed complete consumption of the activated ester and a red spot appeared below the activated ester. The reaction was quenched with TEAB buffer (0.1 M, 10 ml) purified by preparative HPLC.

### General Procedure for Boc Deprotection (Steps B and B′)

Dye-linker was dissolved in 10 mL of 15% TFA in DCM and reacted at room temperature for 3 hrs and reaction was monitored by LC-MS. After reaction was completed, volatiles were removed by reduced pressure and dissolved in the 5 mL 0.1 M TEAB. The resulting solution was evaporated to obtain desired product.

### General Procedure for the Steps D and D′

In an oven dried rounded bottomed flask, fluorene (1 eq), potassium tertbutoxide (2.75 eq) and ethanol were added. The resulting mixture was refluxed then n-carboxybenzaldehyde (1.2 eq) was added potion wise as a solid. The reaction was kept refluxing overnight. The mixture was cooled to room temperature and poured into 10% HCl solution which was cooled in an ice bath. The resulting yellow mixture was extracted with ethyl acetate (x2) and dried with MgSO₄ and concentered in vacuum to yield 9-flurenyl-n′-benzolic acid.

### Procedure for Step F

9,10-dibromoanatharcene (3 g, 8.93 mmol), 3,5-dimethylcarboxy phenyl boronic acid pinacol ester (3.4 g, 10.7 mmol) were dissolved in 40 mL of toluene and the mixture was heated together then sodium carbonate (2.48 g, 26.8 mmol) in 20 mL of water and Pd(PPh₃)₄ (0.5 g, 0.446 mmol) was added. Reaction was heated for 2 days. Reaction was cooled to room temperature and organic layer was separated concentrated in vacuum. The resulting material was purified via silica gel chromatography to yield the desired product.

### Procedure for Step G

Product from the Step F was mixed with NaOH (0.25 g, 6.28 mmol) in 60 ml of Water (10 mL) and THF (50 mL) solution and the resulting solution was refluxed for 16 h. The solution was cooled at room temperature and 10% HCl was added dropwise to acidify the mixture. THF was removed and product was dissolved in DCM. The organic component was washed with water and concentrated in vacuum. The resulting solid was dissolved in methanol, stirred for 2 h, filtered and concentrated in vacuum to yield the desired product (0.332 g, 60%).

### Procedure for the Step I

Product from the Step H (0.26 g, 0.406 mmol), 4-carboxyphenyl boronic acid pinacol ester (0.25 g, 1.02 mmol), sodium carbonate (0.17 g, 1.64 mmol), Pd(PPh₃)₄ (0.025 g, 0.02 mmol), 6 mL of water and 12 ml of DMF were added. The mixture was heated at 90° C. for 16 h. Reaction was cooled to room temperature and acidified with 10% HCl and resulting mixture was concentrated in vacuum. The crude compound was dissolved in acetone, filtered through Celite and concentrated in vacuum. The resulting material was purified via preparative HPLC to yield the desired product (0.103 mmol, 25%).

Compounds 7B, 6B and 4 as well as the corresponding ffCs are characterized as following: Compound 7B reported mass [M-H]⁻ 932.04; C-sPA-Compound 7B reported mass [M-3H]3-611.52; Compound 6B reported mass [M-H]-931.69; C-sPA-Compound 6B reported mass [M-H]-1833.92; Compound 4 reported mass [M-H]-1163.96; C-sPA-Compound 4 reported mass [M-2H]2-1033.68.

### Example 2. Sequencing Experiments on Illumina MiSeq® Platform

The ffC labeled with the new photo protected dyes described herein were tested on Illumina MiSeq® instrument, which was set up to take the first image with a blue excitation light (˜450 nm) and the second image with a green excitation light (˜520 nm). With respect to the two-channel methods described herein, nucleic acids can be sequenced utilizing methods and systems described in U.S. Patent Application No. 2013/0079232, the disclosure of which is incorporated herein by reference in its entirety. The standard Miseq® incorporation mix include the following ffNs: Dark G, T-LN3-AF550POPOS0 (a known green dye), C-sPA-reference dye A, C-LN3-SO7181 (a known red dye), A-sPA-reference dye A, A-sPA-BL-NR550S0 (a known green dye).

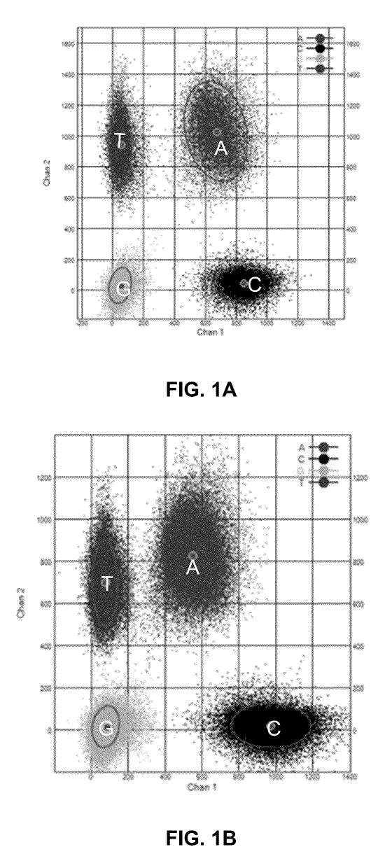

In FIG. 1A, scatterplot was generated using standard MiSeq SBS using an incorporation mix which has 50% C-sPA-reference dye A and 50% C-LN3-SO7181 (a known red dye), the latter was used to control the brightness of the C-cloud to produce a square scatterplot. In FIG. 1B, an incorporation mixture 100% C-sPA-reference dye A was used without C-LN3-SO7181. The scatterplots were generated after six cycles.

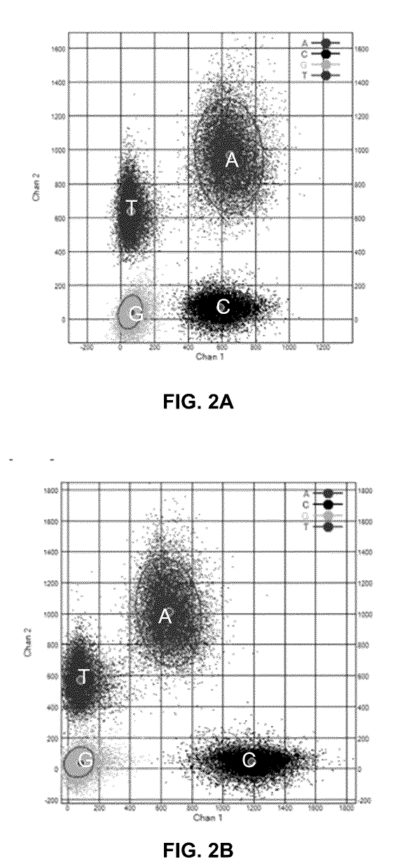

In FIG. 2A, 50% C-sPA-Compound 7B was used in place of the 50% C-sPA-reference dye A in the incorporation mixture described in FIG. 1A. In FIG. 2B, 100% C-sPA-Compound 7B was used in place of the 100% C-sPA-reference dye A in the incorporation mixture described in FIG. 1B. The scatterplots were generated after six cycles.

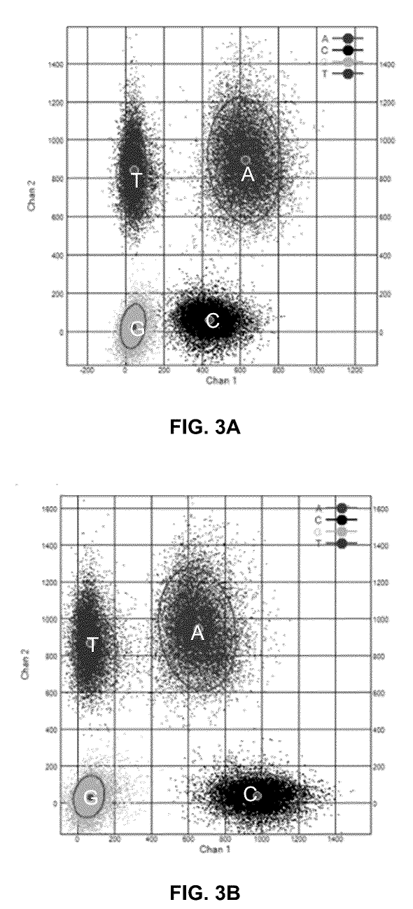

In FIG. 3A, 50% C-sPA-Compound 6B was used in place of the 50% C-sPA-reference dye A in the incorporation mixture described in FIG. 1A. In FIG. 3B, 100% C-sPA-Compound 6B was used in place of the 100% C-sPA-reference dye A in the incorporation mixture described in FIG. 1B. The scatterplots were generated after six cycles. All six experiments were performed within the same SBS run using different positions for the incorporation mixes in the MiSeq® reagent cartridge.

Initial sequencing data shows that the incorporation mixture containing 50% or 100% C-sPA-Compound 7B and C-sPA-Compound 6B were still sufficiently bright and sufficiently incorporated to give good signal (FIGS. 2A, 2B, 3A and 3B).

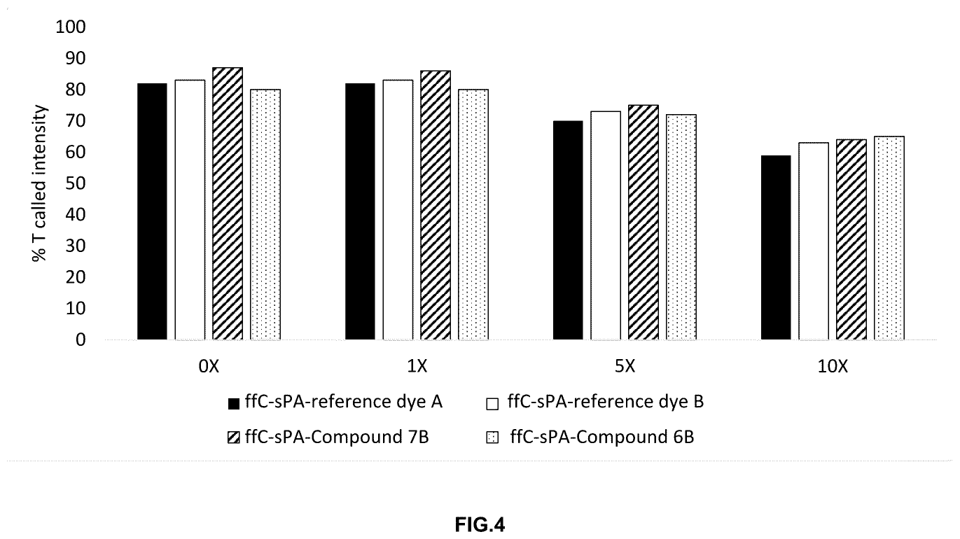

FIG. 4 is a bar chart comparing the % T called signal intensity after 151 cycles of SBS on Illumina's MiSeq® platform at increasing light dosage (0X, 1X, 5X and 10X light dosage change). Four different incorporation mixtures were tested: (1) a standard MiSeq® incorporation mix containing C-sPA-reference dye A; (2) a modified MiSeq® incorporation mix where C-sPA-reference dye A was replaced by C-sPA-reference dye B; (3) a modified MiSeq® incorporation mix where C-sPA-reference dye A was replaced by C-sPA-Compound 7B; and (4) a modified MiSeq® incorporation mix where C-sPA-reference dye A was replaced by C-sPA-Compound 6B. The dosage experiment data indicated that (apart from at lx dosage power) as the cycle number and laser power increases, the decay rates were slower for ffN mixes incorporating Compounds 7B and 6B, indicating that the vinyl substituted fluorene photoprotective moiety improved the fluorescent signal decay rate, and the growing DNA strand was damaged less by these ffNs during each cycle of sequencing.

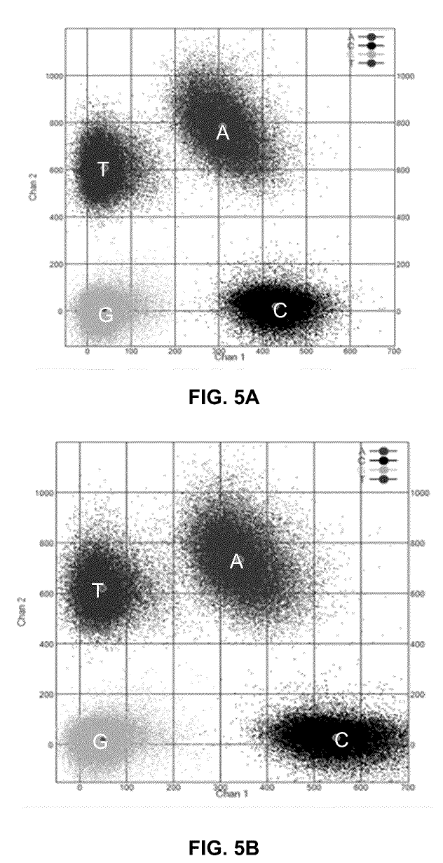

In FIG. 5A, scatterplot was generated using standard MiSeq SBS using an incorporation mix which has 50% C-sPA-reference dye A and 50% C-LN3-SO7181. In FIG. 5B, an incorporation mix containing 100% C-sPA-Compound 4 was used without C-LN3-SO7181. Again, it was observed that the C cloud generated with C-sPA-Compound 4 was brighter than the C cloud generated with C-sPA-reference dye A. Furthermore, it was also observed that the photobleaching rate of the Compound 4 was less than the standard, suggesting that there was a positive impact of the presence of the anthracene photoprotective moiety.

1. A fluorescent compound excitable by a light source having a wavelength between about 400 nm to about 500 nm, wherein the fluorescent compound is covalently attached to one or more photoprotective moieties comprising the formula (Ia), (Ib), (IIa), (IIb), (III) or (IV): 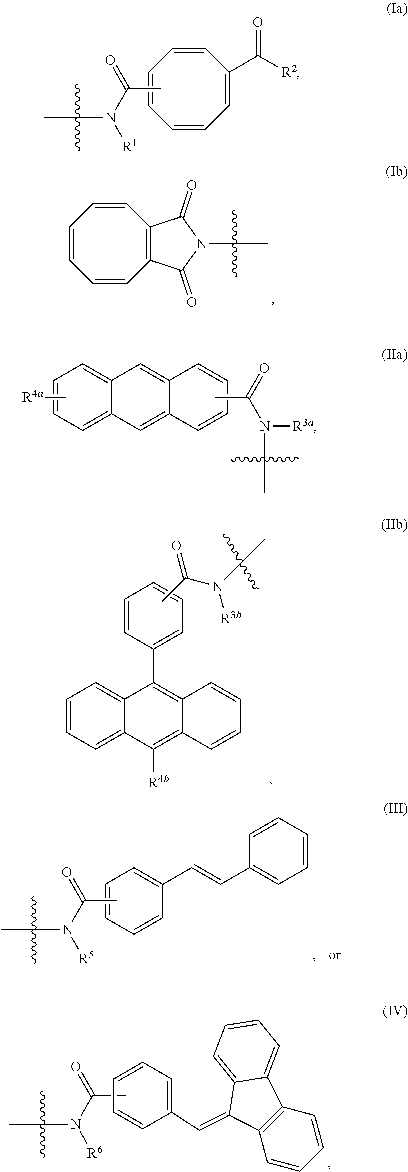
wherein
each of R¹, R^{3a}, R^{3b}, R⁵ and R⁶ is independently H, unsubstituted C₁-C₆ alkyl, or substituted C₁-C₆ alkyl;
R² is —OR⁷, —NR⁸R⁹, —O— or —NR⁸—;
each of R^{4a} and R^{4b} is independently H or optionally substituted C₆-C₁₀ aryl;
each of R⁷, R⁸ and R⁹ is independently H, unsubstituted C₁-C₆ alkyl, substituted C₁-C₆ alkyl, optionally substituted C₆-C₁₀ aryl, optionally substituted C₃-C₇ cycloalkyl, optionally substituted 5 to 10 membered heteroaryl, or optionally substituted 3 to 10 membered heterocyclyl;
each of the aromatic ring or ring system in formulas (Ia), (Ib), (IIa), (IIb), (III) or (IV) is optionally substituted with one, two or three R^{Ar} independently selected from the group consisting of carboxyl, C₁-C₆ alkyl, substituted C₁-C₆ alkyl, C₁-C₆ alkoxy, substituted C₁-C₆ alkoxy, C₁-C₆ halo alkyl, C₁-C₆ halo alkoxy, (C₁-C₆ alkoxy)C₁-C₆ alkyl, —O(C₁-C₆ alkoxy)C₁-C₆ alkyl, optionally substituted amino, amino(C₁-C₆ alkyl), halo, cyano, hydroxy, hydroxy(C₁-C₆ alkyl), nitro, sulfonyl, sulfo, sulfonate, S-sulfonamido, N-sulfonamido, C₆-C₁₀ aryl, 5 to 10 membered heteroaryl, C₃-C₁₀ carbocyclyl, 3 to 10 membered heterocyclyl, —C(═O)OR¹⁰, —C(═O)NR¹¹R¹²; —(CH₂CH₂O)ₙ(C₁-C₆ alkyl), and —O(CH₂CH₂O)ₙ(C₁-C₆ alkyl);
each of R¹⁰, R¹¹ and R¹² is independently H, unsubstituted C₁-C₆ alkyl, or substituted C₁-C₆ alkyl; and
n is an integer of 1 to 20.

2. The fluorescent compound of claim 1, wherein the one or more photoprotective moieties of formula (Ia) is also represented by formula (Ia-1) or (Ia-2): 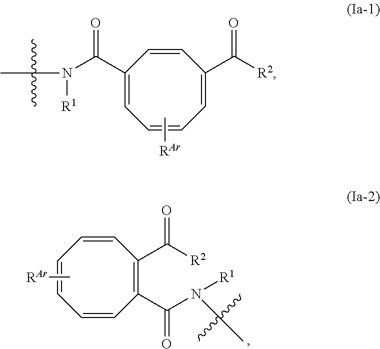
wherein
R¹ is H or substituted C₁-C₆ alkyl;
R² is —OH, —NR⁸ R⁹, or —NR⁸—;
each of R⁸ is independently H or substituted C₁-C₆ alkyl;
R⁹ is unsubstituted or substituted C₁-C₆ alkyl; and
each R^{Ar} is independently carboxyl, sulfo, sulfonate, —O(CH₂CH₂O)ₙCH₃, —C(═O)NH(CH₂)₂₋₅SO₃H, chloro, bromo, nitro, phenyl, C₁-C₆ alkyl, —NH(C₁-C₆ alkyl), or —N(C₁-C₆ alkyl)₂.

3. The fluorescent compound of claim 1, wherein the one or more photoprotective moieties of formula (IIa) is also represented by formula (IIa-1) or (IIa-2): 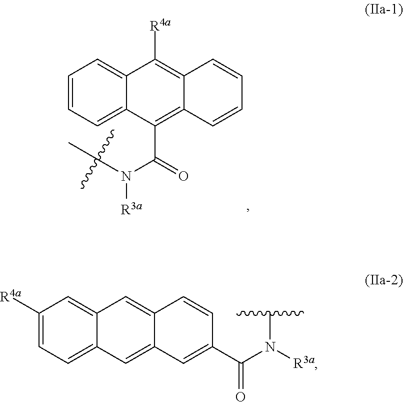
wherein
R^{3a} is H or substituted C₁-C₆ alkyl; and
R^{4a} is H or optionally substituted phenyl.

4. The fluorescent compound of claim 1, wherein the one or more photoprotective moieties of formula (IIb) is also represented by formula (IIb-1): 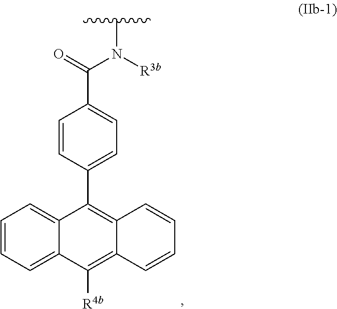
wherein
R^{3b} is H or substituted C₁-C₆ alkyl; and
R^{4b} is H or optionally substituted phenyl.

5. The fluorescent compound of claim 1, wherein the one or more photoprotective moieties of formula (III) is also represented by formula (III-1): 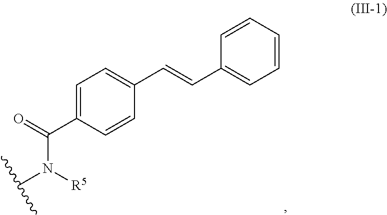
wherein R⁵ is H or substituted C₁-C₆ alkyl.

6. The fluorescent compound of claim 1, wherein the one or more photoprotective moieties of formula (IV) is also represented by formula (IV-1) or (IV-2): 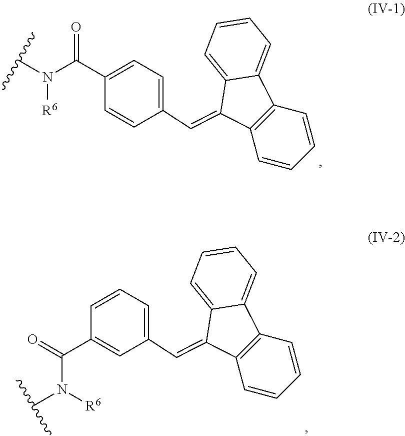
wherein R⁶ is H or substituted C₁-C₆ alkyl.

7. The fluorescent compound of claim 1, wherein the substituted C₁-C₆ alkyl is substituted with one or more of substituents selected from the group consisting of amino, carboxyl, carboxylate, sulfo, sulfonate, and —C(═O)NR¹¹R¹², wherein each of R¹¹ and R¹² is independently H, unsubstituted C₁-C₆ alkyl, or C₁-C₆ alkyl substituted with carboxyl, sulfo, or sulfonate.

8. The fluorescent compound of claim 1, wherein the fluorescent compound is covalently attached to the one or more photoprotective moieties optionally through a linker of formula (V): 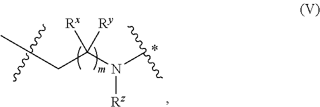
wherein
each R^{x} and R^{y} is independently H, carboxyl, carboxylate, amino, sulfo, sulfonate, —C(O)OR^{a}, or —C(O)NR^{b}R^{c}, or C₁-C₆ alkyl substituted with amino, carboxyl, carboxylate, sulfo, sulfonate, —C(O)OR^{a}, or —C(O)NR^{b}R^{c},
R^{z} is H or C₁-C₆ alkyl substituted with amino, carboxyl, carboxylate, sulfo, sulfonate, —C(O)OR^{a}, or —C(O)NR^{b}R^{c};
R^{a} is optionally substituted C₁-C₆ alkyl, optionally substituted C₆-C₁₀ aryl, optionally substituted 5 to 10 membered heteroaryl, or optionally substituted C₃-C₇ cycloalkyl;
each of R^{b} and R^{c} is independently H, optionally substituted C₁-C₆ alkyl, optionally substituted C₆-C₁₀ aryl, optionally substituted 5 to 10 membered heteroaryl, or optionally substituted C₃-C₇ cycloalkyl;
the carbon atom to which R^{x} and R^{y} are attached in 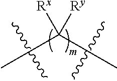
is optionally replaced with O, S, or N, provided that when said carbon atom is replaced with O or S, then R^{x} and R^{y} are both absent; when said carbon atom is replaced with N, then R^{y} is absent; and
m is an integer between 1 and 10;
wherein the asterisk * indicates the attachment point of the linker to the fluorescent compound.

9. The fluorescent compound of claim 8, wherein R^{z} is H.

10. The fluorescent compound of claim 8, wherein m is 1, 2, 3 or 4.

11. The fluorescent compound of claim 8, wherein each of R^{x} and R^{y} is H.

12. The fluorescent compound of claim 8, wherein m is 1, R^{x} is II and R^{y} is carboxyl.

13. The fluorescent compound of claim 8, wherein m is greater than 1, R^{x} is H, and each R^{y} is independently H, amino or carboxyl, wherein at least one R^{y} is carboxyl.

14. The fluorescent compound of claim 8, wherein the asterisk * in formula (V) is connected to a carbonyl group of an amide bond formed by the reaction of an amino group of the photo-protecting moiety with a carboxyl group of the fluorescent compound.

15. The fluorescent compound of claim 1, wherein the fluorescent compound having the moiety: 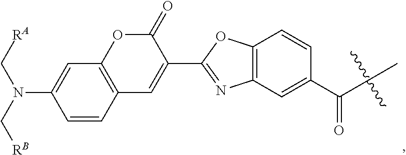
wherein each of R^{A} and R^{B} is independently methyl, or C₁-C₆ alkyl substituted with sulfo, sulfonate, or one or more photoprotective moieties of the formula (Ia), (Ib), (IIa), (IIb), (III) or (IV).

16. The fluorescent compound of claim 1, selected from the group consisting of: 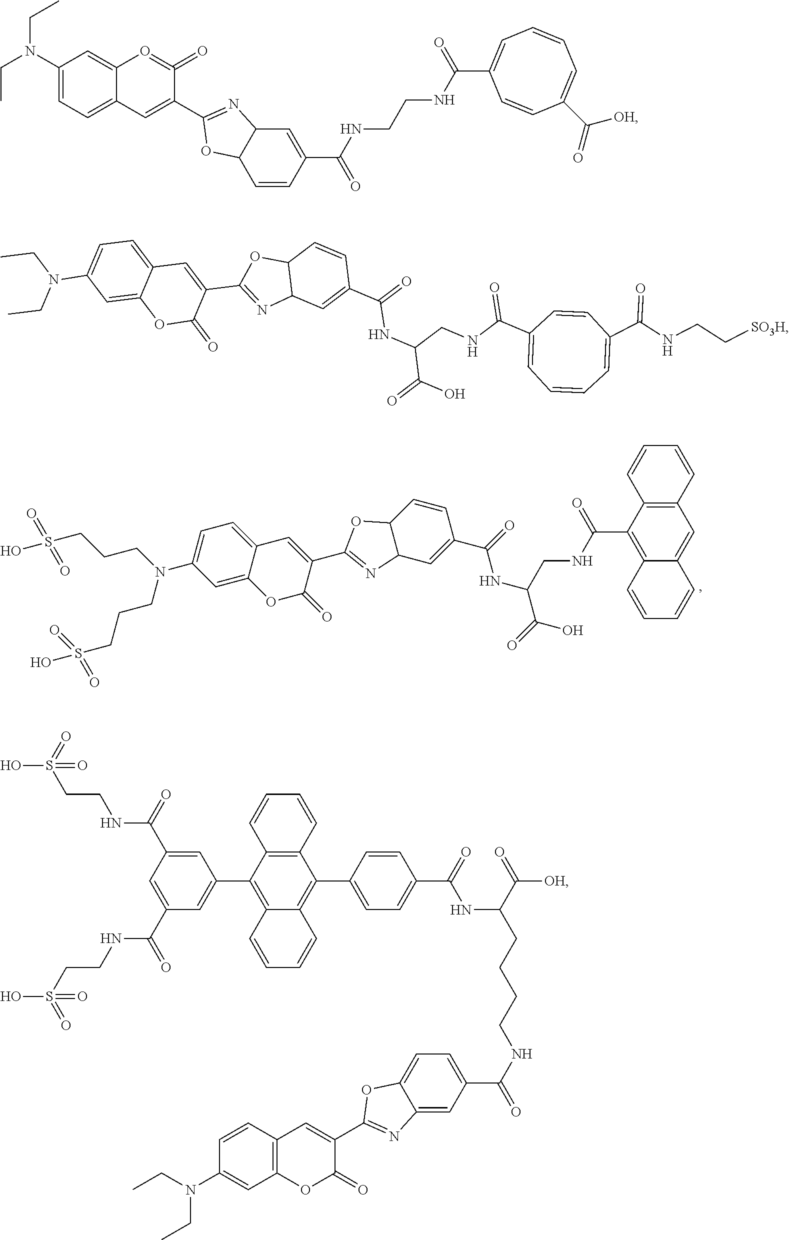 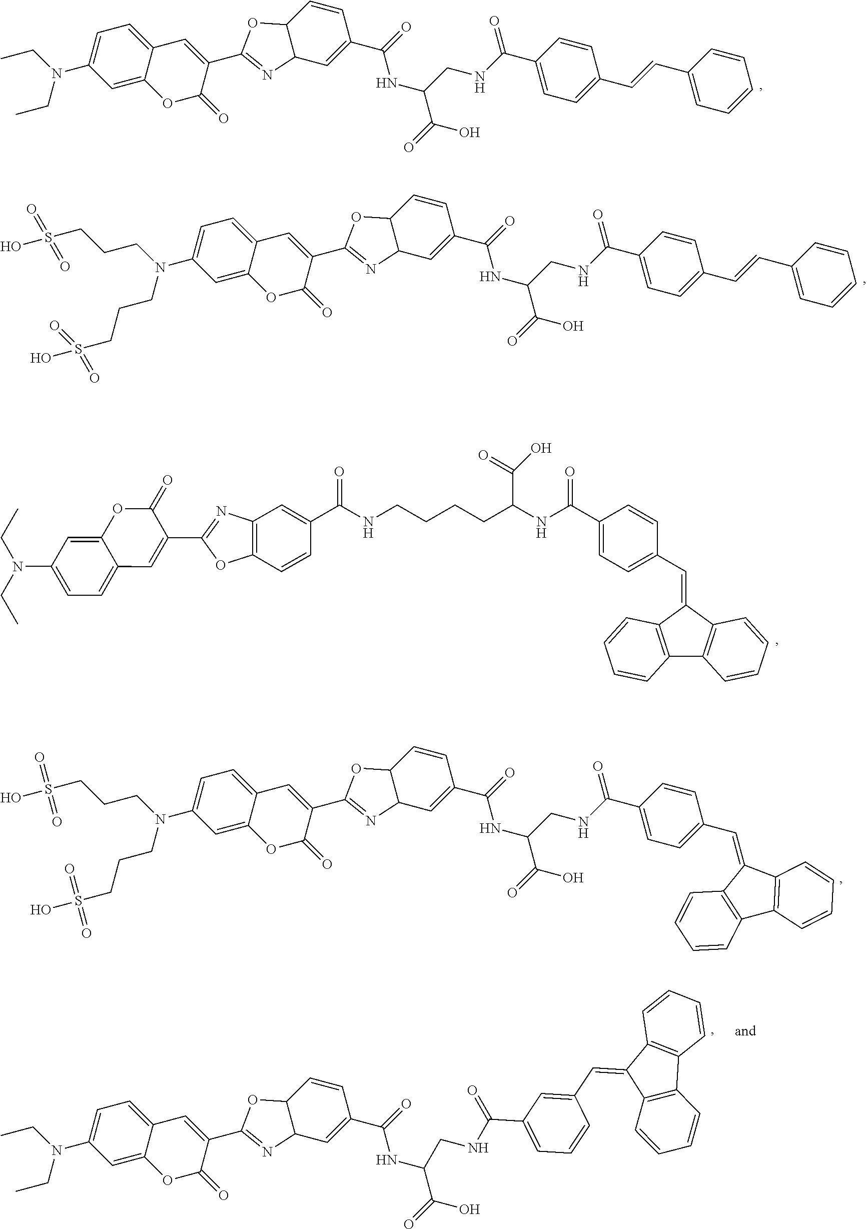 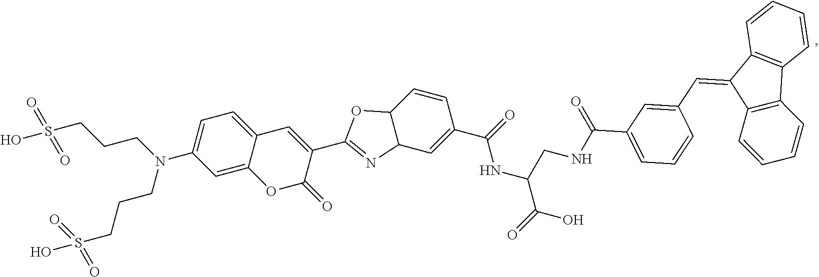 [SMILES: O=C(NC(CNC(=O)c1cccc(C=C2c3ccccc3-c3ccccc32)c1)C(=O)O)C1=CC2N=C(c3cc4ccc(N(CCCS(=O)(=O)O)CCCS(=O)(=O)O)cc4oc3=O)OC2C=C1]
and salts and mesomeric forms thereof.

17. A nucleotide labeled with a fluorescent compound according to claim 1.

18. The nucleotide of claim 17, wherein the fluorescent compound is covalently attached to the nucleotide via a carboxyl group of the fluorescent compound, or a carboxyl group of the photoprotective moiety of the fluorescent compound.

19. The nucleotide of claim 17, wherein the fluorescent compound is attached to the C5 position of a pyrimidine base or the C7 position of a 7-deaza purine base through a linker moiety.

20. The nucleotide of claim 17, further comprising a 3′ blocking group covalently attached to the ribose or deoxyribose sugar of the nucleotide.

21. The nucleotide of claim 20, wherein the nucleotide is a nucleotide triphosphate comprising a 2′ deoxyribose.

22. An oligonucleotide or polynucleotide comprising a nucleotide of claim 17 incorporated therein.

23. The oligonucleotide or polynucleotide of claim 22, wherein the oligonucleotide or polynucleotide is hybridized to at least a portion of a target polynucleotide, and wherein the target polynucleotide is immobilized on a solid support, and the solid support comprises a plurality of different immobilized target polynucleotides.

24. (canceled)

25. (canceled)

26. A kit comprising a first type of nucleotide, which is the labeled nucleotide according to claim 17.

27. The kit of claim 26, further comprising a second type of labeled nucleotide, a third type of labeled nucleotide, and a fourth type of unlabeled nucleotide.

28. The kit of claim 27, wherein each of the first type, second type and the third type of nucleotide is excitable using a light source having a first wavelength.

29. The kit of claim 27, wherein the first type of nucleotide and the third type of nucleotide are excitable using a light source having a first wavelength, the second type of nucleotide and the third type of nucleotide are excitable using a light source having a second wavelength.

30. The kit of claim 28, wherein the first wavelength is from about 450 nm to about 460 nm.

31. (canceled)

32. A method for determining the sequences of a plurality of different target polynucleotides, comprising:
(a) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(b) contacting the solid support with an aqueous solution comprising DNA polymerase and one more of four different types of nucleotides under conditions suitable for DNA polymerase-mediated primer extension, and incorporating one type of nucleotides into the sequencing primers to produce extended copy polynucleotides, wherein at least one type of nucleotide is a nucleotide of claim 21, and wherein each of the four types of nucleotides comprises a 3′ blocking group;
(c) imaging the solid support and performing one or more fluorescent measurements of the extended copy polynucleotides; and
(d) removing the 3′ blocking group of the incorporated nucleotides.

33.-40. (canceled)

41. A method for reducing light-induced sequencing signal decay during sequencing by synthesis, comprising:
(i) contacting a solid support with an incorporation mixture comprising DNA polymerase and one more of four different types of nucleotides, wherein the solid support comprises a plurality of different target polynucleotides immobilized thereon, and sequencing primers that are complementary and hybridized to at least a portion of the target polynucleotides;
(ii) incorporating one type of nucleotides into the sequencing primers to produce extended copy polynucleotides, wherein one or more four types of nucleotides comprises a detectable label, and each of the four types of nucleotides comprises a 3′ blocking group;
(iii) imaging and performing one or more fluorescent measurements of the extended copy polynucleotides in an aqueous scan mixture to determine the identity of the incorporated nucleotides; and
(iv) removing the 3′ blocking groups and the detectable labels of the incorporated nucleotides;
wherein the aqueous scan mixture comprises one or more additives for reducing fluorescent signal decay caused by the fluorescent measurements, and wherein the one or more additives comprise a tri-substituted 1,4-cyclooctatetraene (COT) analog.

42.-50 (canceled)

51. A kit for use with a sequencing apparatus, comprising a scan mixture composition, wherein the scan mixture composition comprises one or more additives for reducing fluorescent signal decay caused by the fluorescent measurements, and wherein the one or more additives comprise a tri-substituted 1,4-cyclooctatetraene (COT) analog.

52.-59. (canceled)
